(19) United States
(12) Patent Application Publication
(10) Pub. No.: US 2024/0254283 A1
(43) Pub. Date: Aug. 1, 2024

(54) BASE-CATALYZED ISOMERIZATION OF MUCONIC ACID DERIVATIVES

(71) Applicant: IOWA STATE UNIVERSITY RESEARCH FOUNDATION, INC., Ames, IA (US)

(72) Inventors: Jean-Philippe TESSONNIER, Ames, IA (US); Brent Howard SHANKS, Ames, IA (US); Eric William COCHRAN, Ames, IA (US); Joseph Eugene HADEL, Ames, IA (US); Marco Nazareno DELL'ANNA, Ames, IA (US); Prerana CARTER, Ames, IA (US); Dustin GANSEBOM, Ames, IA (US)

(21) Appl. No.: 18/027,480

(22) PCT Filed: Sep. 21, 2021
(86) PCT No.: PCT/US2021/051236

§ 371 Date: Mar. 21, 2023

Related U.S. Application Data
(60) Provisional application No. 63/080,959, filed on Sep. 21, 2020.

(51) Int. Cl. C08G 69/42; C07C 51/353

(52) U.S. Cl. CPC C08G 69/42; C07C 51/353

(57) ABSTRACT

The present application is directed to processes for the preparation of compounds of Formulae (I), (IV), and (IVb) as described herein. The application further relates to compounds of Formula (IVb) as described herein. Additional embodiments are directed to a processes for the preparation of polymers of Formulae (X), (XVIII), (XIII), and (XIV) as well as to polymers of Formula (XIII) also described herein.

This application claims priority benefit of U.S. Provisional Patent Application No. 63/080,959, filed Sep. 21, 2020, which is hereby incorporated by reference in its entirety.

This invention was made with government support under grant number DOE Contract No. DE-AC02-05CH11231 and DOE Grant No. DE-EE0008492 awarded by U.S. Department of Energy and under grant number NSF Grant No. CBET1804436 awarded by National Science Foundation. The government has certain rights in the invention.

### FIELD

The present application relates to a base-catalyzed isomerization of muconic acid derivatives.

### BACKGROUND

Muconic acid (MA) is a promising biobased platform intermediate that can be converted into a variety of commodity chemicals and high value-added novel products (Shanks et al., “Bioprivileged Molecules: Creating Value From Biomass,” Green Chem. 19(14):3177-3185 (2017); Khalil et al., “Muconic Acid Isomers as Platform Chemicals and Monomers in the Biobased Economy,” Green Chem. 22(5):1517-1541 (2020)). Obtained primarily through glucose fermentation, cis,cis-muconic acid (ccMA) presents a conjugated unsaturation that allows for a variety of chemistries (Matthiesen et al., “Electrochemical Conversion of Muconic Acid to Biobased Diacid Monomers,” ACS Sustainable Chem. Eng. 4(6):3575-3585 (2016)). For instance, the catalytic hydrogenation of ccMA leads to adipic acid, one of the monomers of Nylon 6,6 (Vardon et al., “Cis,Cis-Muconic Acid: Separation and Catalysis to Bio-Adipic Acid for Nylon-6,6 Polymerization,” Green Chem. 18(11):3397-3413 (2016)). On the other hand, performing a Diels-Alder reaction on MA with ethylene followed by dehydrogenation has shown to produce terephthalic acid, one of the monomers of polyethylene terephthalate (Carraher et al., “Cis,Cis-Muconic Acid Isomerization and Catalytic Conversion to Biobased Cyclic-C6-1,4-Diacid Monomers,” Green Chem. 19(13):3042-3050 (2017)). In addition to potential use as a drop-in replacement for many commercially available species, MA may also be converted into a variety of unsaturated novel chemicals. For instance, electrochemical hydrogenation of MA, as described in the U.S. Pat. No. 10,465,043 to Tessonnier et al., gives an unsaturated product, trans-3-hexenedioic acid (t3HDA).

In the case of t3HDA, thiol-ene radical chemistry can be used to graft thiols to the double bond as reported in U.S. Patent Application Publication No. 2019/0153156 to Matthiesen et al. However, the poor reactivity of its unsaturated bonds coupled with the potential for radical initiators to generate hazardous byproducts limits the usefulness of this option (Lowe, A. B. “Thiol-Ene ‘Click’ Reactions and Recent Applications in Polymer and Materials Synthesis,” Polym. Chem. 1(1):17-36 (2010)). Wohl-Ziegler bromination and Prilezhaev epoxidation are useful for creating reactive intermediates to graft a variety of nucleophiles (Djerassi, C. “Brominations with N-Bromosuccinimide and Related Compounds. The Wohl-Ziegler Reaction,” Chem. Rev. 43(2):271-317 (1948); Kim et al., “MCPBA Epoxidation of Alkenes: Reinvestigation of Correlation between Rate and Ionization Potential,” J. Am. Chem. Soc. 120(37):9513-9516 (1998)). Unfortunately, these reactions require toxic solvents and reactants, which presents issues for scalability. In contrast, the Michael addition pathway allows for selective synthesis of targeted molecules while requiring only a base catalyst (Nair et al., “The Thiol-Michael Addition Click Reaction: A Powerful and Widely Used Tool in Materials Chemistry,” Chem. Mater. 26(1):724-744 (2014)). This robust functionalization technique, however, is only available to alpha-beta unsaturated molecules making t3HDA an unsuitable candidate.

The trans-2-hexenedioic acid (t2HDA) isomer would represent a good alternative since its double bond is shifted to the alpha-beta position. In this case, the beta position may be sufficiently electron deficient as to act as an electrophilic site for Michael addition chemistry. Unfortunately, there is currently no available synthetic method that could make the production of t2HDA commercially feasible (U.S. Patent Application Publication No. 2016/0017381 to Beckham at al.; U.S. Pat. No. 8,592,189 to Burgard et al.).

The present application is directed to overcoming these and other deficiencies in the art.

### SUMMARY

One aspect of the present application relates to a method of preparing a compound of Formula (I):

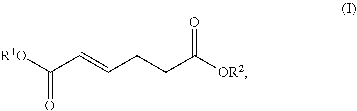

wherein
R¹ is H, D, or C₁₋₆ alkyl;
R² is H, D, or C₁₋₆ alkyl,
or a salt thereof.

This process comprises:
providing a compound of Formula (II) having the structure:

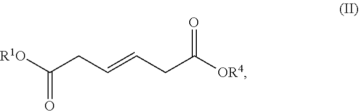

wherein
R³ is H, D, or C₁₋₆ alkyl;
R⁴ is H, D, or C₁₋₆ alkyl, and
forming the compound of Formula (I) from the compound of Formula (II).

Another aspect of the present application relates to a process for preparation of a compound of Formula (IV):

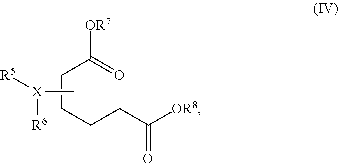

wherein
X is CH, O, S, N, P, or C═O;
R⁵ is selected from the group consisting of H, C₁₋₂₀ alkyl, heteroaryl, heterocyclyl, carborane, C₁₋₂₀ alkyl-O—Zn—O—C₁₋₂₀ alkyl, C₁₋₂₀ alkyl-O—Zn—O-heteroaryl, heteroaryl-O—Zn—O-heteroaryl, C₁₋₂₀ alkyl-O—Zn—O-heterocyclyl, heterocyclyl-O—Zn—O-heterocyclyl, and heterocyclyl-O—Zn—O-heteroaryl, wherein C₁₋₂₀ alkyl, heteroaryl, and heterocyclyl can be optionally substituted 1 to 3 times with R⁹;
R⁶ can be absent and, if present, is H or C₁₋₆ alkyl;
R⁷ is H or C₁₋₆ alkyl;
R⁸ is H or C₁₋₆ alkyl;
R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹¹, P(O)(OR¹⁰)(R¹¹)₂, P(O)(R¹¹)₂, BH₂,

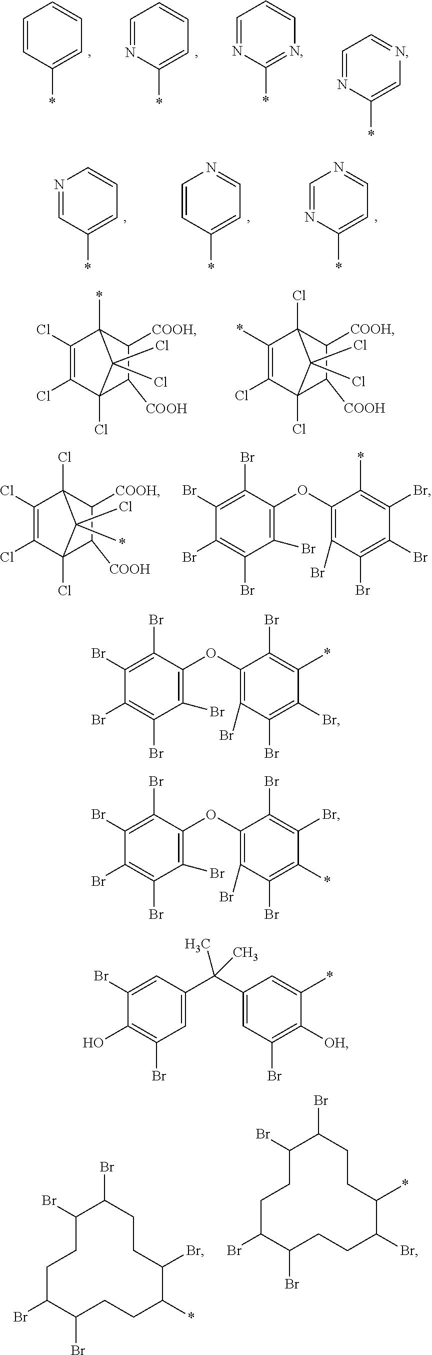

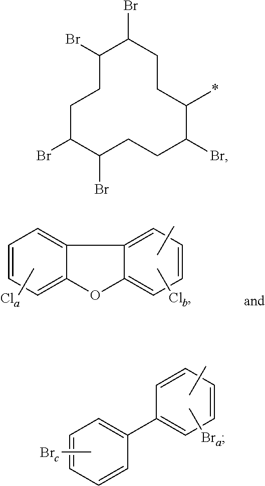

a is 1, 2, 3, or 4;
b is 1, 2, or 3;
c is 1, 2, 3, 4, or 5;
each R¹⁰ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl,
[symbol] is the point of attachment of R⁹ to R⁵;
or a salt thereof.

This process comprises:
providing a compound of Formula (I) prepared according to the process described above, and
forming the compound of Formula (IV) from the compound of Formula (I).

A further aspect of the present application relates to a process for preparation of a compound of Formula (IVb):

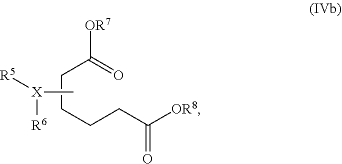

wherein
X is CH, O, S, N, P, or C═O;
R⁵ is selected from the group consisting of H, C₁₋₂₀ alkyl, heteroaryl, heterocyclyl, carborane, C₁₋₂₀ alkyl-O—Zn—O—C₁₋₂₀ alkyl, C₁₋₂₀ alkyl-O—Zn—O-heteroaryl, heteroaryl-O—Zn—O-heteroaryl, C₁₋₂₀ alkyl-O—Zn—O-heterocyclyl, heterocyclyl-O—Zn—O-heterocyclyl, and heterocyclyl-O—Zn—O-heteroaryl, wherein C₁₋₂₀ alkyl, heteroaryl, and heterocyclyl can be optionally substituted 1 to 3 times with R⁹;
R⁶ can be absent and, if present, is H or C₁₋₆ alkyl;
R⁷ is H or C₁₋₆ alkyl;
R⁸ is H or C₁₋₆ alkyl;
R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹¹, P(O)(OR¹⁰)(R¹¹)₂, P(O)(R¹¹)₂, BH₂,

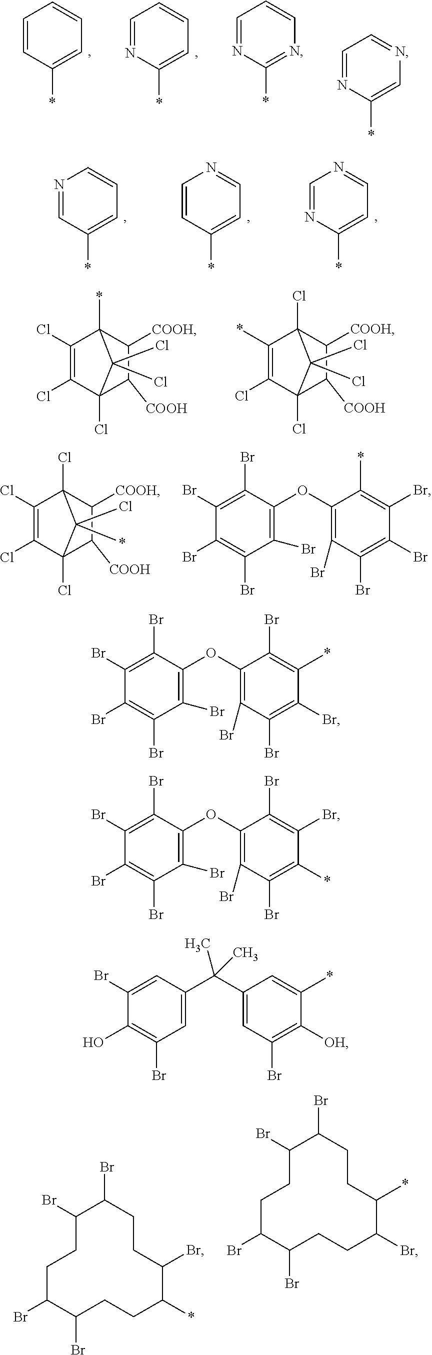

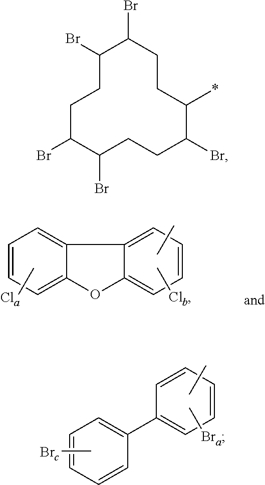

a is 1, 2, 3, or 4;
b is 1, 2, or 3;
c is 1,2,3,4, or 5;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl,
[symbol] is the point of attachment of R⁹ to R⁵;
with the proviso that if R⁷ is H, then R⁸ is not H,
or a salt thereof.

This process comprises:
providing a compound of Formula (VII) having the structure:

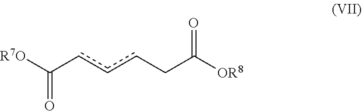

forming the compound of Formula (IVb) from the compound of Formula (VII).

Another aspect of the present application relates to a compound of Formula (IVb):

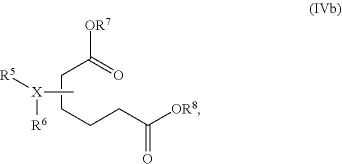

wherein
X is CH, O, S, N, P, or C═O;
R⁵ is selected from the group consisting of H, C₁₋₂₀ alkyl, heteroaryl, heterocyclyl, carborane, C₁₋₂₀ alkyl-O—Zn—O—C₁₋₂₀ alkyl, C₁₋₂₀ alkyl-O—Zn—O-heteroaryl, heteroaryl-O—Zn—O-heteroaryl, C₁₋₂₀ alkyl-O—Zn—O-heterocyclyl, heterocyclyl-O—Zn—O-heterocyclyl, and heterocyclyl—O—Zn—O-heteroaryl, wherein C₁₋₂₀ alkyl, heteroaryl, and heterocyclyl can be optionally substituted 1 to 3 times with R⁹;
R⁶ can be absent and, if present, is H or C₁₋₆ alkyl;
R⁷ is H or C₁₋₆ alkyl;
R⁸ is H or C₁₋₆ alkyl;
R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹¹, P(O)(OR¹⁰)(R¹¹)₂, P(O)(R¹)₂, BH₂,

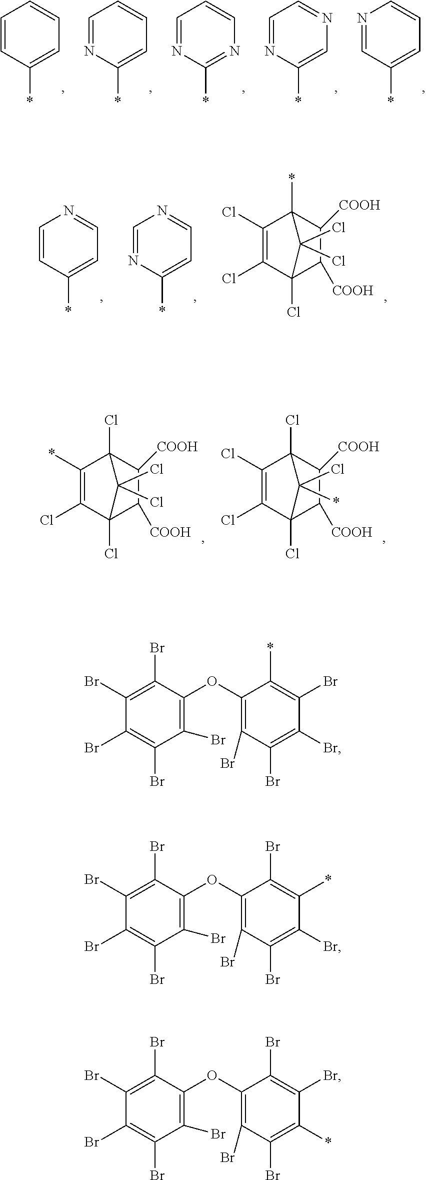

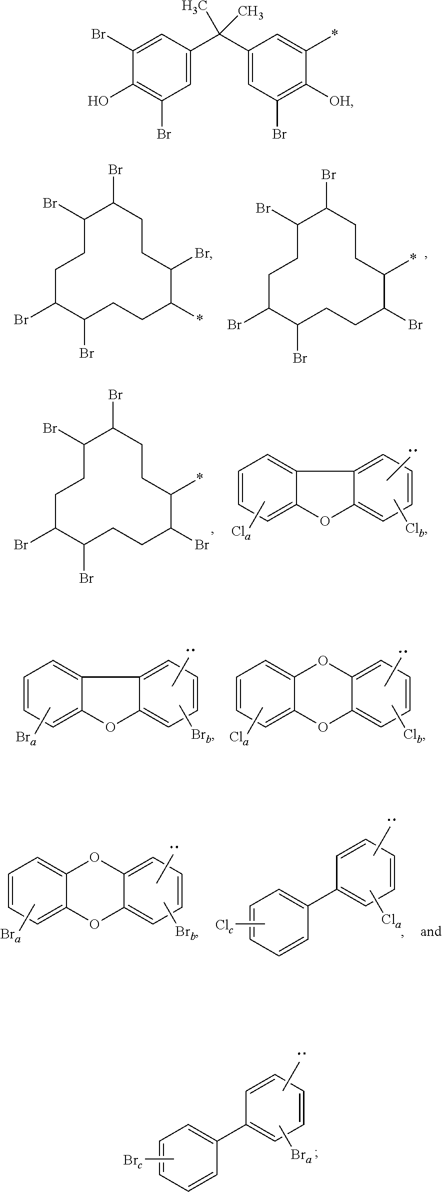

a is 1, 2, 3, or 4;
b is 1, 2, or 3;
c is 1,2,3,4, or 5;
each R¹⁰ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl,
[symbol] is the point of attachment of R⁹ to R⁵;
with the proviso that if R⁷ is H, then R⁸ is not H,
or a salt thereof.

A further aspect of the present application relates to a process of making a polymer of Formula (X):

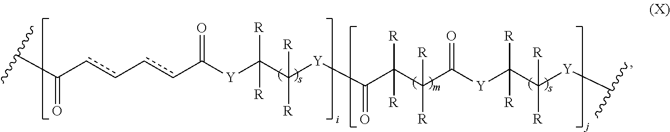

wherein
Y is NH or 0;
R is independently selected from the group consisting of H and C₁₋₂₀ alkyl;
i is 1 to 1,000,000;
j is 1 to 1,000,000;
m is 0 to 32;
s is 0 to 32; and
[symbol] is a terminal group of the polymer; [symbol] is a single or a double bond, and only one of [symbol] is a double bond.

This process comprises:
preparing the compound of Formula (I) by the process described above;
providing a compound having the structure of Formula (XI):

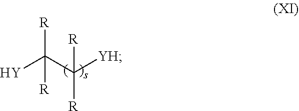

providing a compound having the structure of Formula (XII):

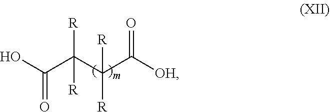

reacting the compound of Formula (I), the compound of Formula (XI), and the compound of Formula (XII) under conditions effective to produce the polymer of Formula (X).

Another aspect of the present application relates to a polymer of Formula (X):

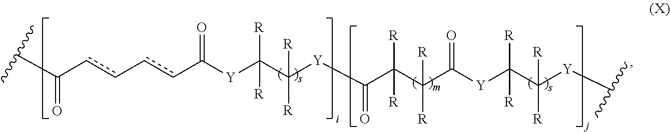

wherein
Y is NH or 0;
R is independently selected from the group consisting of H and C₁₋₂₀ alkyl;
i is 1 to 1,000,000;
j is 1 to 1,000,000;
m is 0 to 32;
s is 0 to 32; and
[symbol] is a terminal group of the polymer;
[symbol] a single or a double bond, and only one of [symbol] is a double bond.

A further aspect of the present application relates to a process of making a polymer of Formula (XVIII):

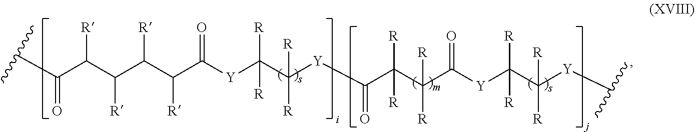

wherein
Y is NH or 0;
R is independently selected from the group consisting of H and C₁₋₂₀ alkyl;
each R′ is independently H or

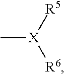

and only one of R′ is

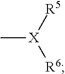

X is CH, O, S, N, P, or C═O;
R⁵ is selected from the group consisting of H, C₁₋₂₀ alkyl, heteroaryl, heterocyclyl, carborane, C₁₋₂₀ alkyl-O—Zn—OC₁₋₂₀ alkyl, C₁₋₂₀ alkyl-O—Zn—O-heteroaryl, heteroaryl-O—Zn—O-heteroaryl, C₁₋₂₀ alkyl-O—Zn—O-heterocyclyl, heterocyclyl—O—Zn—O-heterocyclyl, and heterocyclyl—O—Zn—O-heteroaryl, wherein C₁₋₂₀ alkyl, heteroaryl, and heterocyclyl can be optionally substituted 1 to 3 times with R⁹;
R⁶ can be absent and, if present, is H or C₁₋₆ alkyl;
R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹¹, P(O)(OR¹⁰)(R¹¹)₂, P(O)(R¹¹)₂, BH₂,

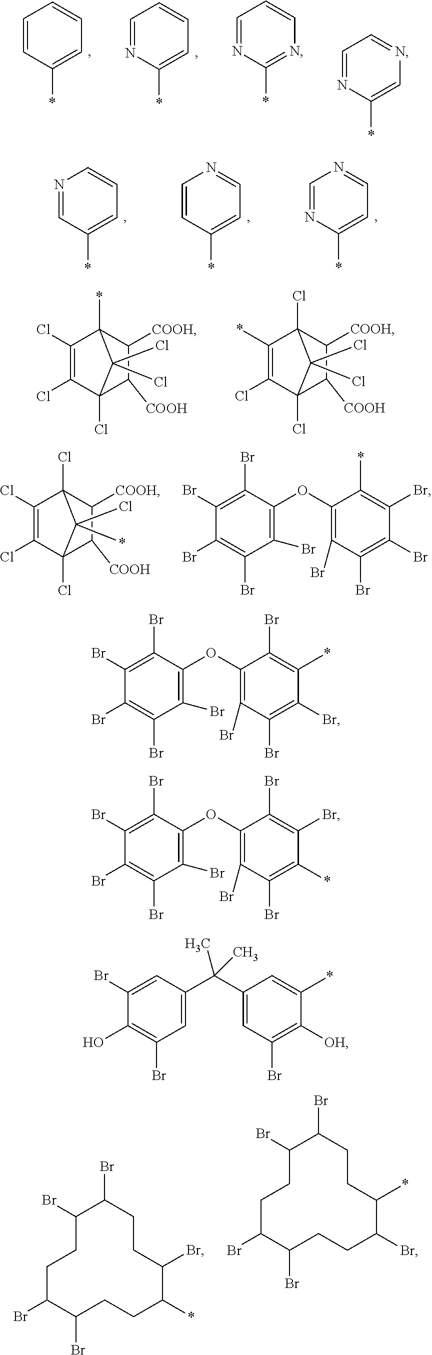

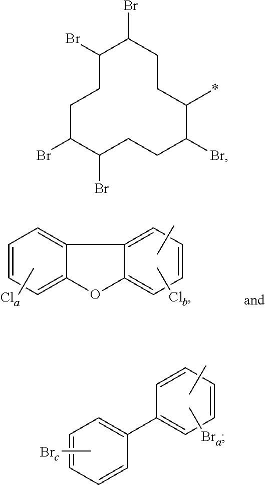

a is 1, 2, 3, or 4;
b is 1, 2, or 3;
c is 1, 2, 3, 4, or 5;
each R¹⁰ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl,
[symbol] is the point of attachment of R⁹ to R⁵;
i is 1 to 1,000,000;
j is 1 to 1,000,000;
m is 0 to 32;
s is 0 to 32; and
[symbol] is a terminal group of the polymer,
this process comprises:
providing the compound of Formula (IVb) as described above;
providing a compound having the structure of Formula (XI):

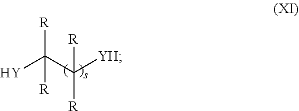

providing a compound having the structure of Formula (XII): HO OH

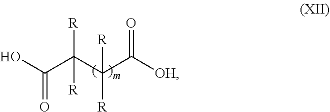

reacting the compound of Formula (IVb), the compound of Formula (XI), and the compound of Formula (XII) under conditions effective to produce the polymer of Formula (XVIII).

Another aspect of the present application relates to a process of making a polymer of Formula (XIII):

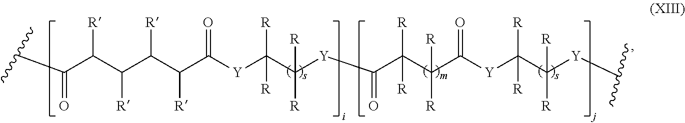

wherein
Y is NH or 0;
R is independently selected from the group consisting of H and C₁₋₂₀ alkyl;
each R′ is independently H or

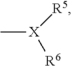

and only one of R′ is

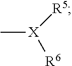

X is CH, O, S, N, P, or C═O;
R⁵ is selected from the group consisting of H, C₁₋₂₀ alkyl, heteroaryl, heterocyclyl, carborane, C₁₋₂₀ alkyl-O—Zn—O—C₁₋₂₀ alkyl, C₁₋₂₀ alkyl-O—Zn—O-heteroaryl, heteroaryl-O—Zn—O-heteroaryl, C₁₋₂₀ alkyl-O—Zn—O-heterocyclyl, heterocyclyl—O—Zn—O-heterocyclyl, and heterocyclyl—O—Zn—O-heteroaryl, wherein C₁₋₂₀ alkyl, heteroaryl, and heterocyclyl can be optionally substituted 1 to 3 times with R⁹;
R⁶ can be absent and, if present, is H or C₁₋₆ alkyl;
R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹¹, P(O)(OR¹⁰)(R¹¹)₂, P(O)(R¹¹)₂, BH₂,

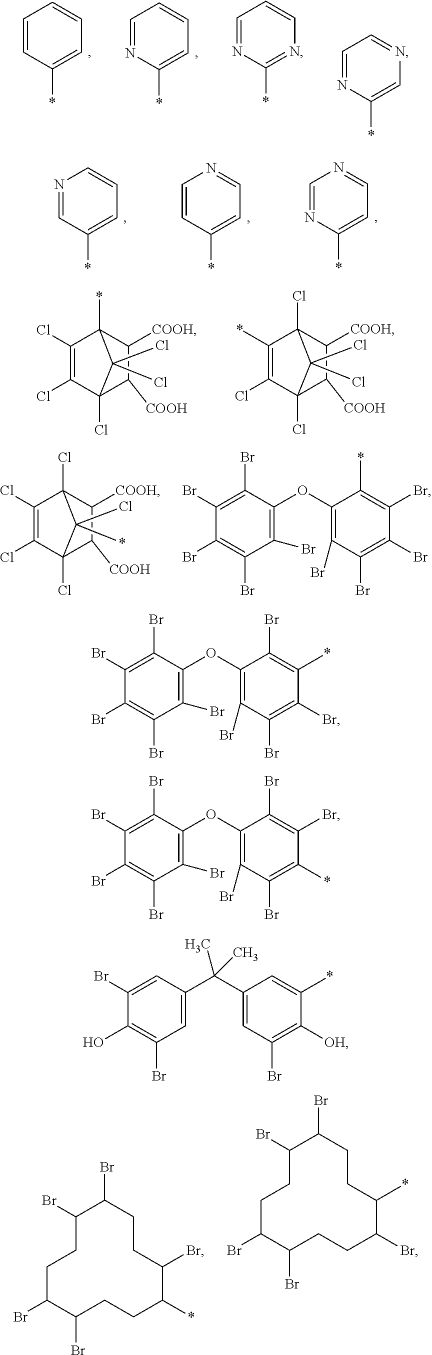

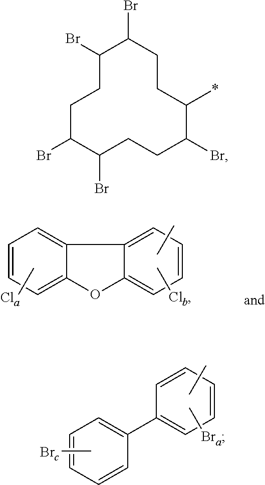

a is 1, 2, 3, or 4;
b is 1, 2, or 3;
c is 1, 2, 3, 4, or 5;
each R¹⁰ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl,
[symbol] is the point of attachment of R⁹ to R⁵;
i is 1 to 1,000,000;
j is 1 to 1,000,000;
m is 0 to 32;
s is 0 to 32; and
[symbol] is a terminal group of the polymer;
[symbol] is a single bond attached to one of the positions shown.

This process comprises:
preparing the compound of Formula (IV) by the process as described above;
providing a compound having the structure of Formula (XI):

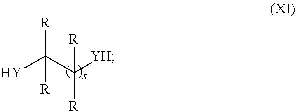

providing a compound having the structure of Formula (XII):

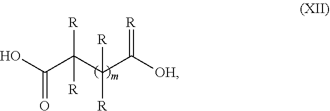

reacting the compound of Formula (IV), the compound of Formula (XI), and the compound of Formula (XII) under conditions effective to produce the polymer of Formula (XIII).

A further aspect of the present application relates to a polymer of Formula (XIII):

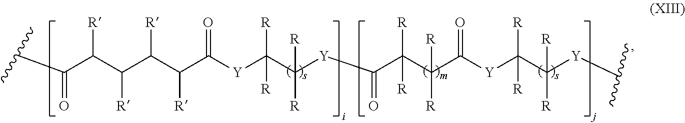

wherein
Y is NH or 0;
R is independently selected from the group consisting of H and C₁₋₂₀ alkyl;
each R′ is independently H or

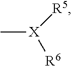

and only one of R′ is

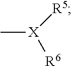

X is CH, O, S, N, P, or C═O;
R⁵ is selected from the group consisting of H, C₁₋₂₀ alkyl, heteroaryl, heterocyclyl, carborane, C₁₋₂₀ alkyl-O—Zn—O—C₁₋₂₀ alkyl, C₁₋₂₀ alkyl-O—Zn—O-heteroaryl, heteroaryl-O—Zn—O-heteroaryl, C₁₋₂₀ alkyl-O—Zn—O-heterocyclyl, heterocyclyl—O—Zn—O-heterocyclyl, and heterocyclyl—O—Zn—O-heteroaryl, wherein C₁₋₂₀ alkyl, heteroaryl, and heterocyclyl can be optionally substituted 1 to 3 times with R⁹;R⁶ can be absent and, if present, is H or C₁₋₆ alkyl;
R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹¹, P(O)(OR¹⁰)(R¹)₂, P(O)(R¹)₂, BH₂,

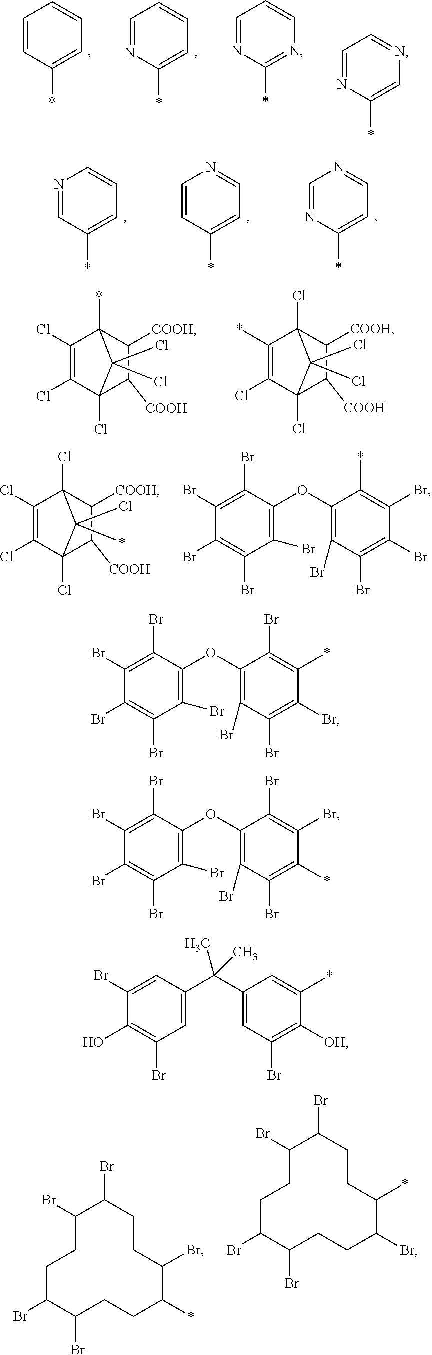

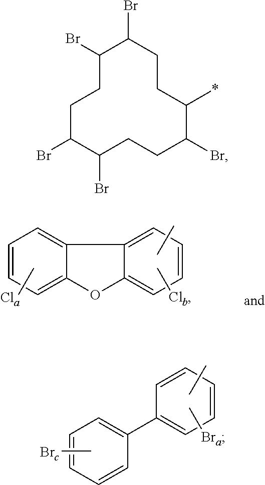

a is 1, 2, 3, or 4;
b is 1, 2, or 3;
c is 1, 2, 3, 4, or 5;
each R¹⁰ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl, [symbol]
[symbol] is the point of attachment of R⁹ to R⁵;
i is 1 to 1,000,000;
j is 1 to 1,000,000;
m is 0 to 32;
s is 0 to 32; and
[symbol] is a terminal group of the polymer;
[symbol] is a single bond attached to one of the positions shown.

Another aspect of the present application relates to a process for preparation of a compound of Formula (XIV):

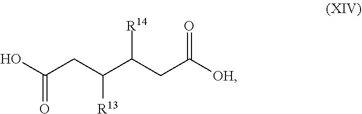

wherein
R¹³ is YR¹⁷R¹⁸;
R¹⁴ is ZR¹⁹R²⁰;
Y and Z are independently selected from CH, O, S, N, P, or C═O;
R¹⁷ and R¹⁹ are independently selected from the group consisting of H, C₁₋₂₀ alkyl, heteroaryl, heterocyclyl, carborane, C₁₋₂₀ alkyl-O—Zn—O C₁₋₂₀ alkyl, C₁₋₂₀ alkyl-O—Zn—O heteroaryl, heteroaryl-O—Zn—O-heteroaryl, C₁₋₂₀ alkyl-O—Zn—O-heterocyclyl, heterocyclyl O—Zn—O heterocyclyl, and heterocyclyl O—Zn—O heteroaryl, wherein C₁₋₂₀ alkyl, heteroaryl, and heterocyclyl can be independently optionally substituted 1 to 3 times with R²¹.
R¹⁸ and R²⁰ independently can be absent and, if present, are independently H or C₁₋₆ alkyl;
R²¹ is independently selected from the group consisting of H, C₁₋₆ alkyl, SH, NH₂, PH₂, P(O)(OR²²)₂, P(O)(OR²²)₃, P(O)(OR²²)₂R²³, P(O)(OR²²)(R²³)₂, P(O)(R²³)₂, BH₂,

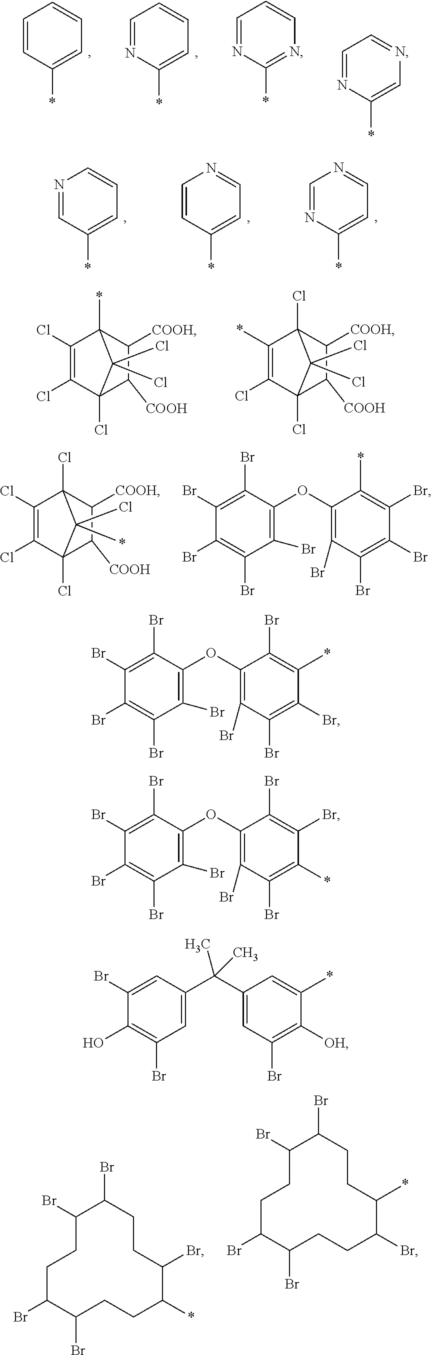

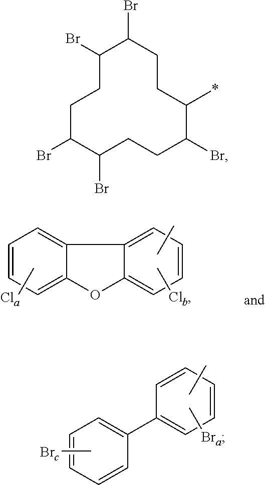

a is 1, 2, 3, or 4;
b is 1, 2, or 3;
c is 1, 2, 3, 4, or 5;
each R²² is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R²³ is independently selected from the group consisting of H, C₁₋₆ alkyl, C₁₋₆ alkenyl, and aryl, wherein aryl can be optionally substituted 1 to 3 times with halogen, or C₁₋₆ alkyl,
[symbol] is the point of attachment of R²¹ to R¹⁷ or R¹⁹ or a salt thereof.

This process comprises:
providing a compound of Formula (XV) having the structure:

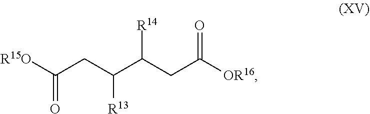

wherein
R¹⁵ is H or C₁₋₆ alkyl;
R¹⁶ is H or C₁₋₆ alkyl; and
forming the compound of Formula (I) from the compound of Formula (II).

In the present application, a simple base-catalyzed method to synthesize t2HDA was demonstrated by using t3HDA produced through electrochemical hydrogenation of muconic acid. t3HDA was chosen as an intermediate as it can be electrochemically produced from MA with 100% yield and high faradaic efficiency (Matthiesen et al., “Electrochemical Conversion of Biologically Produced Muconic Acid: Key Considerations for Scale-Up and Corresponding Technoeconomic Analysis,” ACS Sustainable Chem. Eng. 4(12):7098-7109 (2016), which is hereby incorporated by reference in its entirety). Although previous literature mentioned basic conditions favoring double bond shifts in unsaturated carbonyls, it has not been applied in the context of MA derivatives as to expand available functionalization chemistries (Alcock et al., “On the Conjugative Isomerizations of Beta, Gamma-Unsaturated Esters. Stereochemical Generalizations and Predictions for 1,3-Prototropic Shifts Under Basic Conditions,” J. Org. Chem. 50(19):3526-3535 (1985), which is hereby incorporated by reference in its entirety). This isomerization also circumvents the spontaneous undesired lactonization that is common in MA derivatives (Khalil et al., “Muconic Acid Isomers as Platform Chemicals and Monomers in the Biobased Economy,” Green Chem. 22(5):1517-1541 (2020); Carraher et al., “Cis,Cis-Muconic Acid Isomerization and Catalytic Conversion to Biobased Cyclic-C6-1,4-Diacid Monomers,” Green Chem. 19(13):3042-3050 (2017), which are hereby incorporated by reference in their entirety). In the present application, the effect of the base concentration was analyzed to understand its impact on the performance of the reaction. The isomerization reaction mechanism and the effect of the pH was also demonstrated through isotopic exchange using ¹H NMR.

### BRIEF DESCRIPTION OF THE DRAWINGS

FIGS. 1A-E are graphs showing concentration profiles (FIGS. 1A-D) and reaction scheme of trans-3-hexenedioic acid (t3HDA) isomerization (FIG. 1E). Reaction conditions: 10 g/L t3HDA, temperature 80° C., reaction volume 20 mL, magnetic stirring at 400 rpm, base catalyzed at 1M (FIG. 1A), 5M (FIG. 1B), 10M KOH (FIG. 1C). FIG. 1D: 70 g/L of t3HDA at 1M KOH.

FIGS. 2A-B show t3HDA isomerization reaction (FIG. 2A) and ¹HNMR spectra of t3HDA isomerization reaction in deuterium oxide at pH 14 and different times (FIG. 2B). The reaction was conducted at 80° C., in presence of 1 M KOD and 10 g/L of trans-2-hexenedioic acid (t2HDA). The peaks related to t3HDA (Ha, Hb) and the peaks related to t2HDA are shown.

FIG. 3 is a graph showing the ratio of the areas of peaks Ha and Hb.

FIG. 4 is ¹HNMR spectra of t3DMHDA isomerization. The reaction was conducted at 120° C. in DMF solvent.

FIG. 5 is ¹HNMR spectra of t3DMHDA isomerization. The reaction was conducted at 120° C. using t3DMHDA as solvent and adding 5 weight % DABCO catalyst.

FIGS. 6A-C show the concentration profiles for trans-3-hexenedioic acid (t3HDA) isomerization at different temperatures. Reaction conditions: heat block with oil contact for more uniform temperature distribution, 20 mL reaction volume with 10 g/L t3HDA, temperatures of 25° C. (FIG. 6A), 50° C. (FIG. 6B), and 80° C. (FIG. 6C), magnetic stirring at 400 rpm, base catalyzed with 1M KOH.

FIG. 7 shows the yield of t2HDA for reactions run at different temperatures.

FIGS. 8A-B show the concentration profiles for trans-3-hexenedioic acid (t3HDA) isomerization at higher concentration of the base catalyst. Reaction conditions: heat block with oil contact for more uniform temperature distribution, 20 mL reaction volume with 10 g/L t3HDA, temperature of 25° C. (FIG. 8A), and 80° C. (FIG. 8B), magnetic stirring at 400 rpm, base catalyzed with 10 M KOH.

FIGS. 9A-B show the Arrhenius (FIG. 9A) and Van′t Hoff (FIG. 9B) plots.

FIG. 10 shows GC trace of reaction mixture after 5 days.

FIG. 11 is MS spectra of the product peak and corresponding fragmentation analysis.

FIG. 12 is ¹HNMR spectra in D₂O of salts between diacid monomers and HMDA.

FIG. 13 is ¹HNMR spectra in acetic acid d₄ of nylon 66 made from 50% functionalized nylon salts.

### DETAILED DESCRIPTION

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs. In the event that there is a plurality of definitions for a term herein, those in this section prevail unless stated otherwise.

The term “alkyl” means an aliphatic hydrocarbon group which may be straight or branched having about 1 to about 20 carbon atoms in the chain. Branched means that one or more lower alkyl groups such as methyl, ethyl or propyl are attached to a linear alkyl chain. Exemplary alkyl groups include methyl, ethyl, n-propyl, i-propyl, n-butyl, t-butyl, n-pentyl, and 3-pentyl.

The term “alkylene” refers to a group obtained by removal of a hydrogen atom from an alkyl group. Non-limiting examples of alkylene include methylene and ethylene.

The term “alkenyl” means an aliphatic hydrocarbon group containing a carbon carbon double bond and which may be straight or branched having about 2 to about 10 carbon atoms in the chain. Branched means that one or more lower alkyl groups such as methyl, ethyl, or propyl are attached to a linear alkenyl chain. Exemplary alkenyl groups include ethenyl, propenyl, n-butenyl, and i-butenyl.

The term “aryl” means an aromatic monocyclic or multicyclic ring system of 6 to about 14 carbon atoms, preferably of 6 to about 10 carbon atoms. Representative aryl groups include phenyl and naphthyl.

The term “organohalogen compounds” means organic compounds that contain at least one halogen bonded to carbon. The organohalogen compounds include but are not limited to chlorinated volatile organic compounds (CVOCs), trichloroethylene (TCE), perchloroethylene (PCE), dioxins, polybrominated dibenzo-p-dioxins (PBDD), polybrominated dibenzofurans (PBDF), polybromonated biphenyls (PBB), polychlorinated biphenyls (PCB), polychlorinated dibenzo-p-dioxins (PCDD), polychlorinated dibenzofurans (PCDF), and polychlorinated biphenyls. Suitable organohalogen compounds include chlorobenzene, bromobenzene, carbon tetrachloride, trichloroethane, dichloroethane, and benzyl chloride.

The term “halogen” means fluoro, chloro, bromo, or iodo.

The term “heteroaryl” means an aromatic monocyclic or multicyclic ring system of about 5 to about 14 ring atoms, preferably about 5 to about 10 ring atoms, in which one or more of the atoms in the ring system is/are element(s) other than carbon, for example, nitrogen, oxygen, or sulfur. In the case of multicyclic ring system, only one of the rings needs to be aromatic for the ring system to be defined as “Heteroaryl”. Preferred heteroaryls contain about 5 to 6 ring atoms. The prefix aza, oxa, thia, or thio before heteroaryl means that at least a nitrogen, oxygen, or sulfur atom, respectively, is present as a ring atom. A nitrogen atom of a heteroaryl is optionally oxidized to the corresponding N-oxide. Representative heteroaryls include pyridyl, 2-oxo-pyridinyl, pyrimidinyl, pyridazinyl, pyrazinyl, triazinyl, furanyl, pyrrolyl, thiophenyl, pyrazolyl, imidazolyl, oxazolyl, isoxazolyl, thiazolyl, isothiazolyl, triazolyl, oxadiazolyl, thiadiazolyl, tetrazolyl, indolyl, isoindolyl, benzofuranyl, benzothiophenyl, indolinyl, 2-oxoindolinyl, dihydrobenzofuranyl, dihydrobenzothiophenyl, indazolyl, benzimidazolyl, benzooxazolyl, benzothiazolyl, benzoisoxazolyl, benzoisothiazolyl, benzotriazolyl, benzo[1,3]dioxolyl, quinolinyl, isoquinolinyl, quinazolinyl, cinnolinyl, pthalazinyl, quinoxalinyl, 2,3-dihydro-benzo[1,4]dioxinyl, benzo[1,2,3]triazinyl, benzo[1,2,4]triazinyl, 4H-chromenyl, indolizinyl, quinolizinyl, 6aH-thieno[2,3-d]imidazolyl, 1H-pyrrolo[2,3-b]pyridinyl, imidazo[1,2-a]pyridinyl, pyrazolo[1,5-a]pyridinyl, [1,2,4]triazolo[4,3-a]pyridinyl, [1,2,4]triazolo[1,5-a]pyridinyl, thieno[2,3-b]furanyl, thieno[2,3-b]pyridinyl, thieno[3,2-b]pyridinyl, furo[2,3-b]pyridinyl, furo[3,2-b]pyridinyl, thieno[3,2-d]pyrimidinyl, furo[3,2-d]pyrimidinyl, thieno[2,3-b]pyrazinyl, imidazo[1,2-a]pyrazinyl, 5,6,7,8-tetrahydroimidazo[1,2-a]pyrazinyl, 6,7-dihydro-4H-pyrazolo[5,1-c][1,4]oxazinyl, 2-oxo-2,3-dihydrobenzo[d]oxazolyl, 3,3-dimethyl-2-oxoindolinyl, 2-oxo-2,3-dihydro-1H-pyrrolo[2,3-b]pyridinyl, benzo[c][1,2,5]oxadiazolyl, benzo[c][1,2,5]thiadiazolyl, 3,4-dihydro-2H-benzo[b][1,4]oxazinyl, 5,6,7,8-tetrahydro-[1,2,4]triazolo[4,3-a]pyrazinyl, [1,2,4]triazolo[4,3-a]pyrazinyl, 3-oxo-[1,2,4]triazolo[4,3-a]pyridin-2(3H)-yl, and the like.

As used herein, “heterocyclyl” refers to a stable 3—to 18-membered ring (radical) which consists of carbon atoms and from one to five heteroatoms selected from the group consisting of nitrogen, oxygen, and sulfur. For purposes of this application, the heterocycle may be a monocyclic, or a polycyclic ring system, which may include fused, bridged, or spiro ring systems; and the nitrogen, carbon, or sulfur atoms in the heterocycle may be optionally oxidized; the nitrogen atom may be optionally quaternized; and the ring may be partially or fully saturated. Examples of such heterocycles include, without limitation, azepinyl, azocanyl, pyranyl dioxanyl, dithianyl, 1,3-dioxolanyl, tetrahydrofuryl, dihydropyrrolidinyl, decahydroisoquinolyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, 2-oxoazepinyl, oxazolidinyl, oxiranyl, piperidinyl, piperazinyl, 4-piperidonyl, pyrrolidinyl, pyrazolidinyl, thiazolidinyl, tetrahydropyranyl, thiamorpholinyl, thiamorpholinyl sulfoxide, and thiamorpholinyl sulfone. Further heterocycles and heteroaryls are described in Katritzky et al., eds., Comprehensive Heterocyclic Chemistry: The Structure, Reactions, Synthesis and Use of Heterocyclic Compounds, Vol. 1-8, Pergamon Press, N.Y. (1984), which is hereby incorporated by reference in its entirety.

The term “monocyclic” used herein indicates a molecular structure having one ring.

The term “polycyclic” or “multi-cyclic” used herein indicates a molecular structure having two or more rings, including, but not limited to, fused, bridged, or spiro rings.

The term “substituted” or “substitution” of an atom means that one or more hydrogen on the designated atom is replaced with a selection from the indicated group, provided that the designated atom's normal valency is not exceeded.

“Unsubstituted” atoms bear all of the hydrogen atoms dictated by their valency. When a substituent is keto (i.e., =0), then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious therapeutic agent.

The term “optionally substituted” is used to indicate that a group may have a substituent at each substitutable atom of the group (including more than one substituent on a single atom), provided that the designated atom's normal valency is not exceeded and the identity of each substituent is independent of the others. Up to three H atoms in each residue are replaced with alkyl, halogen, haloalkyl, hydroxy, loweralkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy. “Unsubstituted” atoms bear all of the hydrogen atoms dictated by their valency. When a substituent is keto (i.e., =0), then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious therapeutic agent.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. The present application is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

The term “salts” means the inorganic, and organic base addition salts, of compounds of the present application. Suitable metal salts include the sodium, potassium, calcium, barium, zinc, magnesium, and aluminum salts.

The term “copolymer” refers to a polymer derived from more than one species of monomer.

The term “statistically defined manner” refers to the repeat unit sequence distribution (RUSD) of the polymer, which is determined by the polymerization chemistry, the number and nature of co-monomers, and the reaction conditions under which the polymer is formed. For any polymer, the RUSD can be represented by a probability function Pᵢ(j) that indicates the likelihood that the identity of the repeat unit at location j along the chain contour is i. Common RUSD classifications include, but are not limited to, random (Pᵢ=constant) and block (e.g., Pᵢ(j<f) 0 and Pᵢ(j≥f) 1 and given fixed contour coordinate ƒ). RUSD prediction and measurement are discussed in most polymer chemistry texts (e.g., Hiemenz and Lodge, Polymer Chemistry, 2^{nd} Ed., Boca Raton Fl., CRC Press (2007), which is hereby incorporated by reference in its entirety).

The term “alternating copolymer” or “alternating polymer” refers to a copolymer consisting of two or more species of monomeric units that are arranged in an alternating sequence (in which every other building unit is different (—M₁M₂—)ₙ.

The term “random copolymer” or “random polymer” refers to a copolymer in which there is no definite order for the sequence of the different building blocks (—M₁M₂M₁M₁M₂M₁M₂M₂—).

The term “statistical copolymer” or “statistical polymer” refers to a copolymer in which the sequential distribution of the monomeric units obeys known statistical laws.

The term “block copolymer” or “block polymer” refers to a macromolecule consisting of long sequences of different repeat units. Exemplary block polymers include, but are not limited to AₙBₘ, AₙBₘAₘ, AₙBₘCₖ, or AₙBₘCₖAₙ.

One aspect of the present application relates to a process for preparation of a compound of Formula (I):

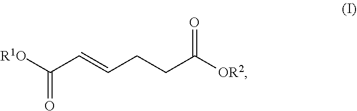

wherein
R¹ is H, D, or C₁₋₆ alkyl;
R² is H, D, or C₁₋₆ alkyl,
or a salt thereof,
this process comprises:
providing a compound of Formula (II) having the structure:

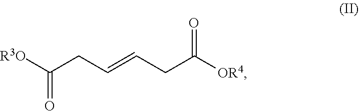

wherein
R³ is H, D, or C₁₋₆ alkyl;
R⁴ is H, D, or C₁₋₆ alkyl, and
forming the compound of Formula (I) from the compound of Formula (II).

In one embodiment of the process for preparation of a compound of Formula (I), R¹, R², R³, and R⁴ are each selected independently from H, D, or Me.

In another embodiment of the process for preparation of a compound of Formula (I), the step of forming the compound of Formula (I) comprises reacting the compound of Formula (II) in the presence of a base to produce the compound of Formula (I).

In one embodiment of the process for preparation of a compound of Formula (I), the base is selected from the group consisting of alkali hydroxides, primary amines, secondary amines, and tertiary amines.

In another embodiment of the process for preparation of a compound of Formula (I), the base is selected from the group consisting of potassium hydroxide, sodium hydroxide, dimethyl formamide, 1,4-diazabicyclo[2.2.2]octene, triethylamine, 1,8-diazabicyclo[5.4.0]undec-7-ene, 1,5-diazabicyclo[4.3.0]non-5-ene 4-dimethylaminopyridine, 1-methylimidazole, and 1,1,3,3-tetramethylguanidine.

In another embodiment of the process for preparation of a compound of Formula (I), the concentration of the base ranges from 0.1M to 10M.

In another embodiment of the process for preparation of a compound of Formula (I), the concentration of the base ranges from 1M to 5M.

In another embodiment of the process for preparation of a compound of Formula (I), providing a compound of Formula (II) comprises providing a compound of Formula (III) having the structure:

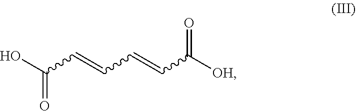

wherein the geometry around each double bond is independently either cis- or trans-, and forming the compound of Formula (II) from the compound of Formula (III).

In another embodiment of the process for preparation of a compound of Formula (I), the compound of Formula (III) has the structure of Formula (IIIa):

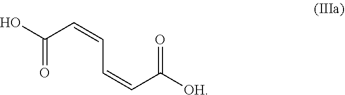

In another embodiment of the process for preparation of a compound of Formula (I), the compound of Formula (III) has the structure of Formula (IIIb):

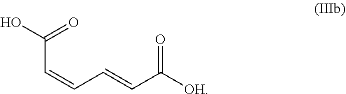

In another embodiment of the process for preparation of a compound of Formula (I), the step of forming the compound of Formula (II) comprises reacting the compound of Formula (III) with a hydrogen source to produce the compound of Formula (II).

In another embodiment of the process for preparation of a compound of Formula (I), the hydrogen source is selected from the group consisting of hydrogen, water, H₃O⁺, sodium borohydride, and lithium aluminum hydride.

In another embodiment of the process for preparation of a compound of Formula (I), the compound of Formula (II) is produced by electrochemical hydrogenation of the compound of Formula (III).

Forming the compound of Formula (II) is performed by saturation/rearrangement of the double bonds of the compound of Formula (III) via reaction with a hydrogen source to arrive at the compound of Formula (II). The hydrogen source may be selected from hydrogen, water, H₃O⁺, sodium borohydride, and lithium aluminum hydride. Forming the compound of Formula (II) is also produced by electrochemical hydrogenation of the compound of Formula (III).

In the case of hydrogen source being hydrogen, the reaction may be carried out with various catalysts such as Pd/C, Wilkinson's catalyst, Lindlar catalyst, and PtO₂. The hydrogenation reaction can be carried out without solvent or in a variety of solvents, for example methanol, ethanol, and isopropanol. The hydrogenation reaction can be carried out at atmospheric pressure or at an elevated pressure, for example about 5 bar, about 10 bar, about 15 bar, about 20 bar, about 25 bar, about 30 bar, about 35 bar, about 40 bar, about 45 bar, about 50 bar, or at a pressure between any two of these values. The hydrogenation reaction can be carried out at ambient temperature or at an elevated temperature, for example about 50° C., about 80° C., about 100° C., about 150° C., about 200° C., about 250° C., about 300° C., or at a temperature between any two of these values.

In the case of hydrogen source being water or H₃O⁺, the reaction can be carried out in a variety of solvents, for example tetrahydrofuran, 1,4-dioxane, methanol, ethanol, and isopropanol. The reaction can be carried out at various temperatures, for example, about −10° C., about 0° C., about 10° C., about 25° C., about 50° C., about 100° C., about 150° C., about 200° C., about 250° C., about 300° C., or at a temperature between any two of these values.

In the case of hydrogen source being sodium borohydride or lithium aluminum hydride, the reaction can be carried out in a variety of solvents, for example tetrahydrofuran, diethyl ether, 1,4-dioxane, methanol, ethanol, and isopropanol. The reaction can be carried out at various temperatures, for example, about −78° C., about −50° C., about −25° C., about −10° C., about 0° C., about 10° C., about 25° C., about 50° C., about 100° C., about 150° C., about 200° C., about 250° C., about 300° C., or at a temperature between any two of these values.

The electrochemical hydrogenation may be carried out in in an electrochemical system comprising an anode, a cathode, and a power source. The electrochemical hydrogenation may be carried out in out in a variety of solvents, for example methanol, ethanol, and isopropanol. The electrochemical hydrogenation may be carried out in out with a variety of proton sources such as ammonium chloride and ammonium acetate.

Another aspect of the present application relates to a process for preparation of a compound of Formula (IV):

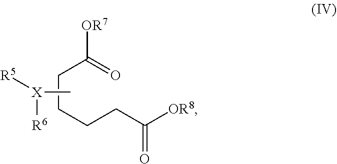

wherein
X is CH, O, S, N, P, or C═O;
R⁵ is selected from the group consisting of H, C₁₋₂₀ alkyl, heteroaryl, heterocyclyl, carborane, C₁₋₂₀ alkyl-O—Zn—OC₁₋₂₀ alkyl, C₁₋₂₀ alkyl-O—Zn—O-heteroaryl, heteroaryl-O—Zn-0heteroaryl, C₁₋₂₀ alkyl-O—Zn—O-heterocyclyl, heterocyclyl—O—Zn—O-heterocyclyl, and heterocyclyl—O—Zn—O-heteroaryl, wherein C₁₋₂₀ alkyl, heteroaryl, and heterocyclyl can be optionally substituted 1 to 3 times with R⁹;
R⁶ can be absent and, if present, is H or C₁₋₆ alkyl;
R⁷ is H or C₁₋₆ alkyl;
R⁸ is H or C₁₋₆ alkyl; R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹¹, P(O)(OR¹⁰)(R¹¹)₂, P(O)(R¹¹)₂, BH₂,

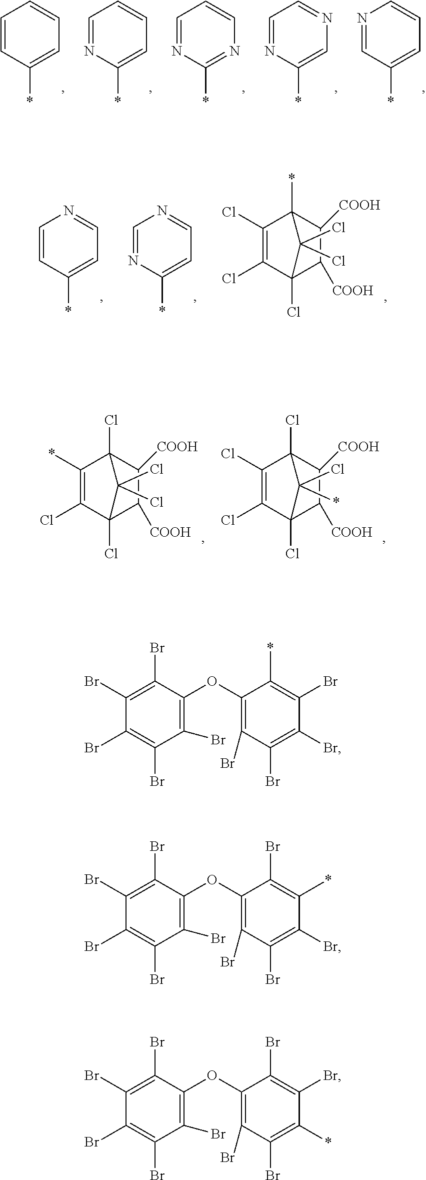

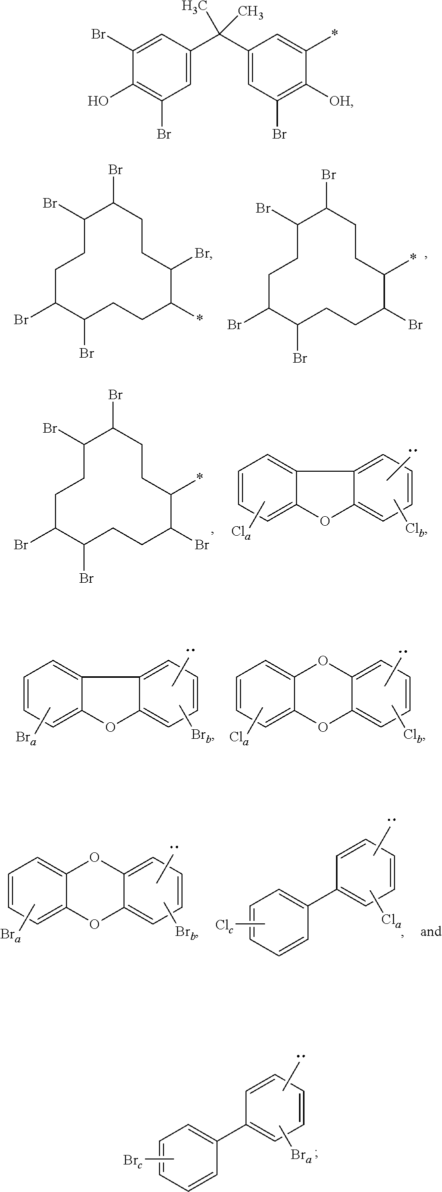

a is 1, 2, 3, or 4;
b is 1, 2, or 3;
c is 1,2,3,4, or 5;
each R¹⁰ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl,

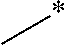 [SMILES: *C]

is the point of attachment of R⁹ to R⁵;
or a salt thereof,
this process comprises:
providing a compound of Formula (I) prepared according to the process described above, and
forming the compound of Formula (IV) from the compound of Formula (I).

In another embodiment of the process for preparation of a compound of Formula (IV), R⁵ is

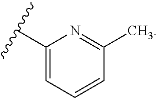 [SMILES: *c1cccc(C)n1]

In another embodiment of the process for preparation of a compound of Formula (IV), R⁵ is selected from the group consisting of

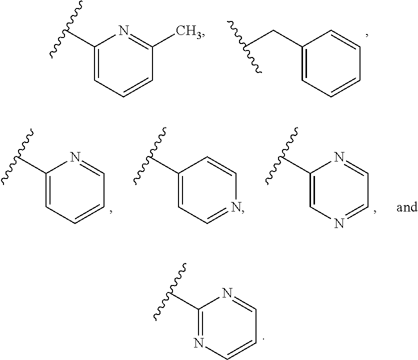 [SMILES: *c1cccc(C)n1 ; *Cc1ccccc1 ; *c1ccccn1 ; *c1ccncc1 ; *c1cnccn1 ; *c1ncccn1]

In another embodiment of the process for preparation of a compound of Formula (IV), the step of forming the compound of Formula (IV) comprises reacting the compound of Formula (I) with a compound of Formula (V):

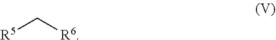

In another embodiment of the process for preparation of a compound of Formula (IV), the step of forming the compound of Formula (IV) comprises reacting the compound of Formula (I) with a compound of Formula (VI):

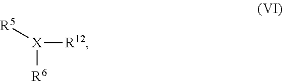

wherein R¹² is H or a suitable leaving group (LG).

In another embodiment of the process for preparation of a compound of Formula (IV), the step of forming the compound of Formula (IV) comprises reacting the compound of Formula (I) with a compound of Formula (VIa):

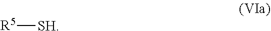

In another embodiment, in the process for preparation of a compound of Formula (IV), the step of reacting the compound of Formula (I) with a compound of Formula (VIa) is conducted in the presence of a radical initiator. The reaction can be performed in a variety of solvents, for example in DMF or DMSO. The reaction can be carried out at ambient temperature or at an elevated temperature, for example about 40° C., about 80° C., about 100° C., about 120° C., about 140° C., about 160° C., about 180° C., or at a temperature between any two of these values. The reaction can be carried out for about 1 hour, about 4 hours, about 8 hours, about 12 hours, about 24 hours, about 36 hours, about 48 hours, about 60 hours, about 72 hours, or for a period between any two of these values.

In another embodiment of the process for preparation of a compound of Formula (IV), the radical initiator is selected from the group consisting of tert-amyl peroxybenzoate, 4,4-azobis(4-cyanovaleric acid), 1,1′-azobis(cyclohexanecarbonitrile), 2,2′-azobisisobutyronitrile (AIBN), benzoyl peroxide, 2,2-bis(tert-butylperoxy)butane, 1,1-bis(tertbutylperoxy)cyclohexane, 2,5-bis(tert-butylperoxy)-2,5-dimethylhexane, 2,5-bis(tert-butylperoxy)-2,5-dimethyl-3-hexyne, bis(1-(tert-butylperoxy)-1-methylethyl)benzene, 1,1-bis(tert-butylperoxy)-3,3,5-trimethylcyclohexane, tert-butyl hydroperoxide, tert-butyl peracetate, tert-butyl peroxide, tert-butyl peroxybenzoate, tert-butylperoxy isopropyl carbonate, cumene hydroperoxide, cyclohexanone peroxide, dicumyl peroxide, lauroyl peroxide, 2,4-pentanedione peroxide, peracetic acid, and potassium persulfate.

In another embodiment, in the process for preparation of a compound of Formula (IV), the step of reacting the compound of Formula (I) with a compound of Formula (VIa) is conducted in dimethylformamide, dimethylsulfoxide, phenol, benzyl alcohol, cresols, ethylene glycol, propylene glycol, halogenated solvents, ethers, and ionic liquids or combination thereof.

In another embodiment of the process for preparation of a compound of Formula (IV), the process further comprises reacting the compound of Formula (IV) with a base to form a compound of Formula (IVa):

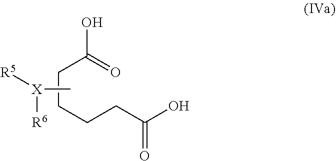

or a salt thereof.

In another embodiment of the process for preparation of a compound of Formula (IV), the base is selected from the group consisting of alkali hydroxides, primary amines, secondary amines, and tertiary amines.

In another embodiment of the process for preparation of a compound of Formula (IV), the base is selected from the group consisting of potassium hydroxide, sodium hydroxide, dimethyl formamide, 1,4-diazabicyclo[2.2.2]octene, triethylamine, 1,8-diazabicyclo[5.4.0]undec-7-ene, 1,5-diazabicyclo[4.3.0]non-5-ene 4-dimethylaminopyridine, 1-methylimidazole, and 1,1,3,3-tetramethylguanidine.

Another aspect of the present application relates to a process for preparation of a compound of Formula (IVb):

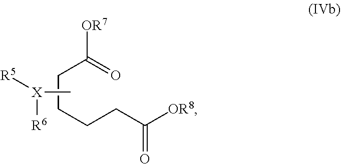

wherein
X is CH, O, S, N, P, or C═O;
R⁵ is selected from the group consisting of H, C₁₋₂₀ alkyl, heteroaryl, heterocyclyl, carborane, C₁₋₂₀ alkyl-O—Zn—O—C₁₋₂₀ alkyl, C₁₋₂₀ alkyl-O—Zn—O-heteroaryl, heteroaryl-O—Zn—O-heteroaryl, C₁₋₂₀ alkyl-O—Zn—O-heterocyclyl, heterocyclyl—O—Zn—O-heterocyclyl, and heterocyclyl—O—Zn—O-heteroaryl, wherein C₁₋₂₀ alkyl, heteroaryl, and heterocyclyl can be optionally substituted 1 to 3 times with R⁹;
R⁶ can be absent and, if present, is H or C₁₋₆ alkyl;
R⁷ is H or C₁₋₆ alkyl;
R⁸ is H or C₁₋₆ alkyl;
R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹¹, P(O)(OR¹⁰)(R¹¹)₂, P(O)(R¹¹)₂, BH₂,

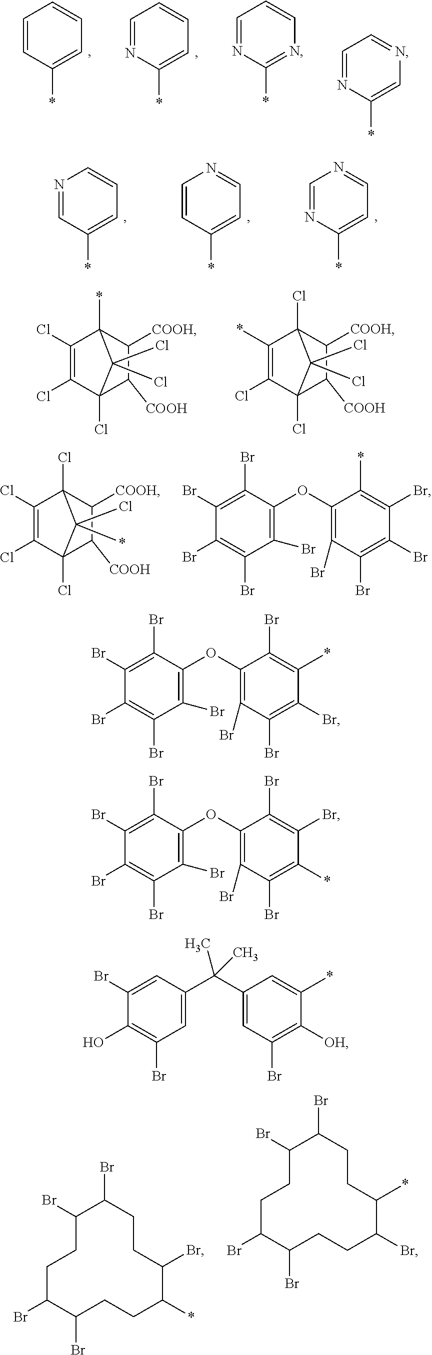

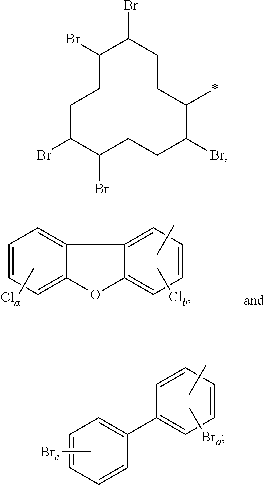

a is 1, 2, 3, or 4;
b is 1, 2, or 3;
c is 1,2,3,4, or 5;
each R¹⁰ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl,
[symbol] is the point of attachment of R⁹ to R⁵;
with the proviso that if R⁷ is H, then R⁸ is not H,
or a salt thereof.

This process comprises:
providing a compound of Formula (VII) having the structure:

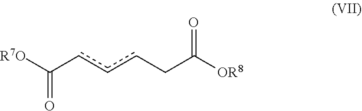

### (VII) and

forming the compound of Formula (IVb) from the compound of Formula (VII).

In another embodiment of the process for preparation of a compound of Formula (IVb), R⁵ is

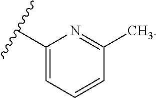 [SMILES: *c1cccc(C)n1]

In another embodiment of the process for preparation of a compound of Formula (IVb), R⁵ is selected from the group consisting of

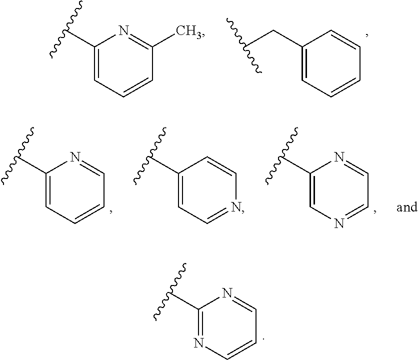 [SMILES: *c1cccc(C)n1 ; *Cc1ccccc1 ; *c1ccccn1 ; *c1ccncc1 ; *c1cnccn1 ; *c1ncccn1]

In another embodiment of the process for preparation of a compound of Formula (IVb), the compound of Formula (VII) has a Formula (VIII):

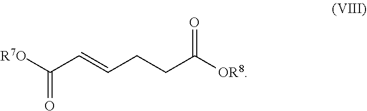

In another embodiment of the process for preparation of a compound of Formula (IVb), the compound of Formula (VII) has a Formula (IX):

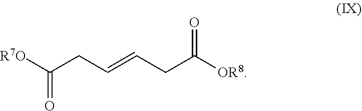

In another embodiment of the process for preparation of a compound of Formula (IVb), the step of forming the compound of Formula (IVb) comprises reacting the compound of Formula (VII) with a compound of Formula (V):

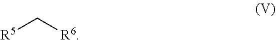

In another embodiment of the process for preparation of a compound of Formula (IVb), the step of forming the compound of Formula (IVb) comprises reacting the compound of Formula (VII) with a compound of Formula (VI):

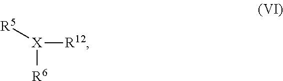

wherein R¹² is H or a suitable leaving group (LG).

In another embodiment of the process for preparation of a compound of Formula (IVb), the step of forming the compound of Formula (IVb) comprises reacting the compound of Formula (VII) with a compound of Formula (VIa):

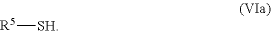

In another embodiment, the process for preparation of a compound of Formula (IVb), the step of reacting the compound of Formula (VII) with a compound of Formula (VIa) is conducted in the presence of a radical initiator. The reaction can be performed in a variety of solvents, for example in DMF or DMSO. The reaction can be carried out at ambient temperature or at an elevated temperature, for example about 40° C., about 80° C., about 100° C., about 120° C., about 140° C., about 160° C., about 180° C., or at a temperature between any two of these values. The reaction can be carried out for about 1 hour, about 4 hours, about 8 hours, about 12 hours, about 24 hours, about 36 hours, about 48 hours, about 60 hours, about 72 hours, or for a period between any two of these values.

In another embodiment of the process for preparation of a compound of Formula (IVb), the radical initiator is selected from the group consisting of tert-amyl peroxybenzoate, 4,4-azobis(4-cyanovaleric acid), 1,1′-azobis(cyclohexanecarbonitrile), 2,2′-azobisisobutyronitrile (AIBN), benzoyl peroxide, 2,2-bis(tert-butylperoxy)butane, 1,1-bis(tertbutylperoxy)cyclohexane, 2,5-bis(tert-butylperoxy)-2,5-dimethylhexane, 2,5-bis(tert-butylperoxy)-2,5-dimethyl-3-hexyne, bis(1-(tert-butylperoxy)-1-methylethyl)benzene, 1,1-bis(tert-butylperoxy)-3,3,5-trimethylcyclohexane, tert-butyl hydroperoxide, tert-butyl peracetate, tert-butyl peroxide, tert-butyl peroxybenzoate, tert-butylperoxy isopropyl carbonate, cumene hydroperoxide, cyclohexanone peroxide, dicumyl peroxide, lauroyl peroxide, 2,4-pentanedione peroxide, peracetic acid, and potassium persulfate.

In another embodiment for the process for preparation of a compound of Formula (IVb), the step of reacting the compound of Formula (VII) with a compound of Formula (VI) is conducted in dimethylformamide, dimethylsulfoxide, phenol, benzyl alcohol, cresols, ethylene glycol, propylene glycol, halogenated solvents, ethers, and ionic liquids or combination thereof.

In another embodiment, the process for preparation of a compound of Formula (IVb) comprises reacting the compound of Formula (IVb) with a base to form a compound of Formula (IVa):

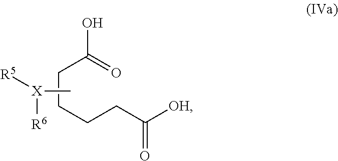

or a salt thereof.

In another embodiment of the process for preparation of a compound of Formula (IVb), the base is selected from the group consisting of alkali hydroxides, primary amines, secondary amines, and tertiary amines.

In another embodiment of the process for preparation of a compound of Formula (IVb), the base is selected from the group consisting of potassium hydroxide, sodium hydroxide, dimethyl formamide, 1,4-diazabicyclo[2.2.2]octene, triethylamine, 1,8-diazabicyclo[5.4.0]undec-7-ene, 1,5-diazabicyclo[4.3.0]non-5-ene 4-dimethylaminopyridine, 1-methylimidazole, and 1,1,3,3-tetramethylguanidine.

One aspect of the present application relates to a compound of Formula (IVb):

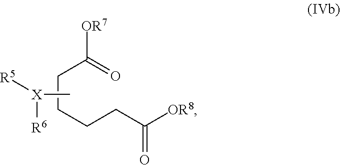

wherein
X is CH, O, S, N, P, or C═O;
R⁵ is selected from the group consisting of H, C₁₋₂₀ alkyl, heteroaryl, heterocyclyl, carborane, C₁₋₂₀ alkyl-O—Zn—O—C₁₋₂₀ alkyl, C₁₋₂₀ alkyl-O—Zn—O-heteroaryl, heteroaryl-O—Zn—O-heteroaryl, C₁₋₂₀ alkyl-O—Zn—O-heterocyclyl, heterocyclyl—O—Zn—O-heterocyclyl, and heterocyclyl—O—Zn—O-heteroaryl, wherein C₁₋₂₀ alkyl, heteroaryl, and heterocyclyl can be optionally substituted 1 to 3 times with R⁹;
R⁶ can be absent and, if present, is H or C₁₋₆ alkyl;
R⁷ is H or C₁₋₆ alkyl;
R⁸ is H or C₁₋₆ alkyl;
R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹¹, P(O)(OR¹⁰)(R¹¹)₂, P(O)(R¹)₂, BH₂,

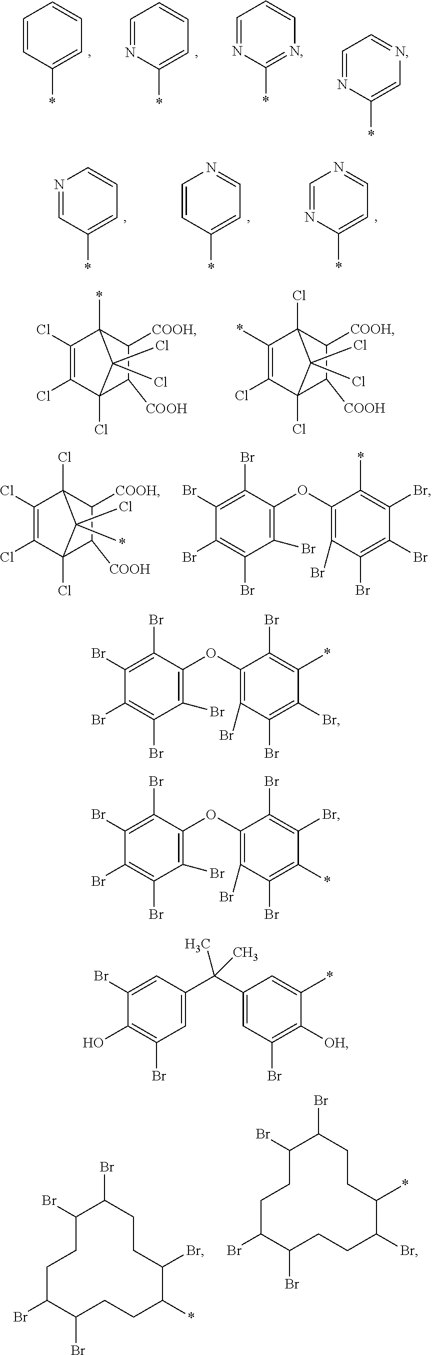

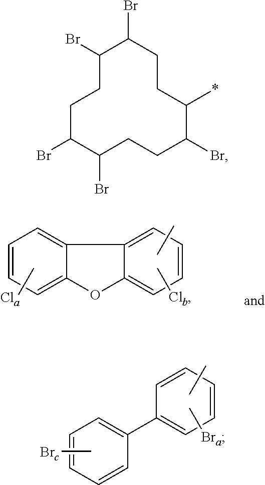

a is 1, 2, 3, or 4;
b is 1, 2, or 3;
c is 1,2,3,4, or 5;
each R¹⁰ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl,
[symbol] is the point of attachment of R⁹ to R⁵;
with the proviso that if R⁷ is H, then R⁸ is not H,
or a salt thereof.

One aspect of the present application relates to a process of making a polymer comprising the moiety:

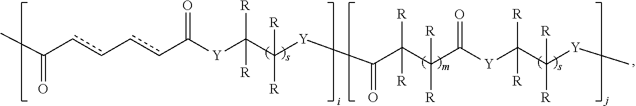

wherein
Y is NH or 0;
R is independently selected from the group consisting of H and C₁₋₂₀ alkyl;
[symbol] is 1 to 1,000,000;
j is 1 to 1,000,000;
m is 0 to 32;
s is 0 to 32; and [symbol]
[symbol] is a single or a double bond, and only one of—is a double bond; this process comprises:
preparing the compound of Formula (I) by the process described above;
providing a compound having the structure of Formula (XI):

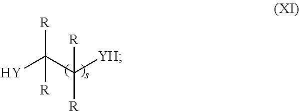

providing a compound having the structure of Formula (XII):

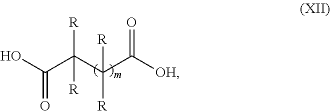

reacting the compound of Formula (I), the compound of Formula (XI), and the compound of Formula (XII) under conditions effective to produce the polymer.

One aspect of the present application relates to a process of making a polymer of Formula (X):

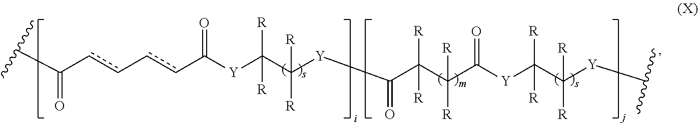

wherein
Y is NH or 0;
R is independently selected from the group consisting of H and C₁₋₂₀ alkyl;
i is 1 to 1,000,000;
j is 1 to 1,000,000;
m is 0 to 32;
s is 0 to 32;and
[symbol] is a terminal group of the polymer;
[symbol] is a single or a double bond, and only one of [symbol] is a double bond;
this process comprises:
preparing the compound of Formula (I) by the process described above;
providing a compound having the structure of Formula (XI):

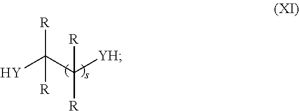

providing a compound having the structure of Formula (XII):

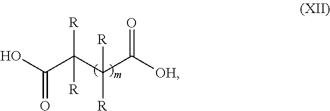

reacting the compound of Formula (I), the compound of Formula (XI), and the compound of Formula (XII) under conditions effective to produce the polymer of Formula (X).

In one embodiment, the step of reacting the compound of formula (I), the compound of formula (XI), and the compound of formula (XII) comprises:
reacting the compound of formula (I) with the compound of formula (XI) to form a salt 1;
reacting the compound of formula (XII) with the compound of formula (XI) to form a salt 2; and
reacting the salt 1 with the salt 2 under conditions effective to produce the polymer.

In another embodiment, the step of reacting the salt 1 with the salt 2 comprises heating the salt 1 with the salt 2 under inert atmosphere in a reaction vessel. In one embodiment, the heating process is conducted under pressure.

During the process of making a polymer according to the present application, salt 1 and salt 2 can be used in any amount from 1 to 99%. In some embodiments, salt 1 and salt 2 are mixed at the ratio of 5% of salt 1 and 95% of salt 2, 10% of salt 1 and 90% of salt 2, 15% of salt 1 and 85% of salt 2, 20% of salt 1 and 80% of salt 2, 25% of salt 1 and 75% of salt 2, 30% of salt 1 and 70% of salt 2, 35% of salt 1 and 65% of salt 2, 40% of salt 1 and 60% of salt 2, 45% of salt 1 and 55% of salt 2, 50% of salt 1 and 50% of salt 2, 55% of salt 1 and 45% of salt 2, 60% of salt 1 and 40% of salt 2, 65% of salt 1 and 35% of salt 2, 70% of salt 1 and 30% of salt 2, 75% of salt 1 and 25% of salt 2, 80% of salt 1 and 20% of salt 2, 85% of salt 1 and 15% of salt 2, 90% of salt 1 and 10% of salt 2, or 95% of salt 1 and 5% of salt 2.

In another embodiment of the process for preparation of a compound of Formula (X), the polymer of Formula (X) has the structure of Formula (Xa):

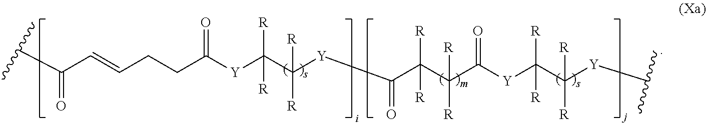

In another embodiment of the process for preparation of a compound of Formula (X), the polymer of Formula (X) has the structure of Formula (Xb):

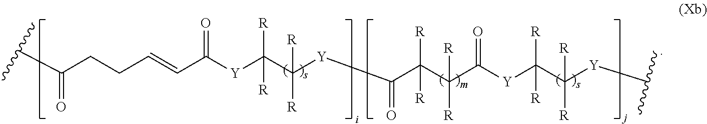

In another embodiment of the process for preparation of a compound of Formula (X), the polymer of Formula (X) has the structure of Formula (Xc):

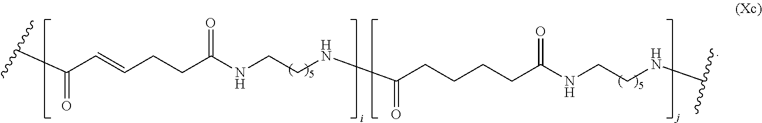

In another embodiment of the process for preparation of a compound of Formula (X), the polymer of Formula (X) has the structure of Formula (Xd):

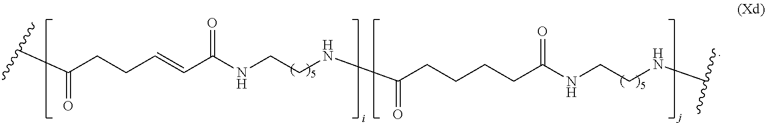

One aspect of the present application relates to a polymer comprising the moiety:

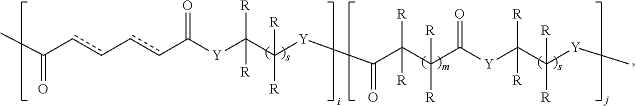

wherein
Y is NH or 0;
R is independently selected from the group consisting of H and C₁₋₂₀ alkyl;
i is 1 to 1,000,000;
j is 1 to 1,000,000;
m is 0 to 32;
s is 0 to 32; and
[symbol] is a single or a double bond, and only one of [symbol] is a double bond.

One aspect of the present application relates to a polymer of Formula (X):

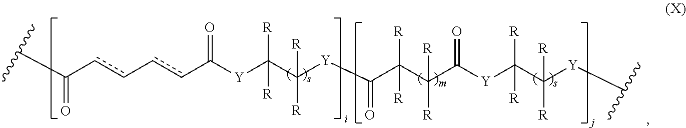

wherein
Y is NH or 0;
R is independently selected from the group consisting of H and C₁₋₂₀ alkyl;
i is 1 to 1,000,000;
j is 1 to 1,000,000;
m is 0 to 32;
s is 0 to 32; and
[symbol] is a terminal group of the polymer;
[symbol] is a single or a double bond, and only one of [symbol] is a double bond.

In another embodiment of the polymer of Formula (X), the polymer has the structure of Formula (Xa), Formula (Xb), Formula (Xc), and Formula (Xd).

In another embodiment of the polymer of Formula (X), the polymer is a statistical polymer.

In another embodiment of the polymer of Formula (X), the polymer is a random polymer.

In another embodiment of the polymer of Formula (X), the polymer is an alternating polymer.

In another embodiment of the polymer of Formula (X), the polymer is a block polymer.

One aspect of the present application relates to a process of making a polymer comprising a moiety:

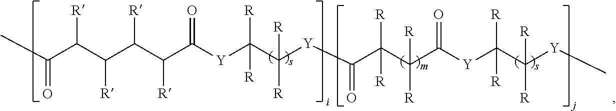

wherein
Y is NH or 0;
R is independently selected from the group consisting of H and C₁₋₂₀ alkyl;
each R′ is independently H or

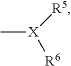

and only one of R′ is

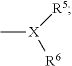

X is CH, O, S, N, P, or C═O;
R⁵ is selected from the group consisting of H, C₁₋₂₀ alkyl, heteroaryl, heterocyclyl, carborane, C₁₋₂₀ alkyl-O—Zn—OC₁₋₂₀ alkyl, C₁₋₂₀ alkyl-O—Zn—O-heteroaryl, heteroaryl-O—Zn—O-heteroaryl, C₁₋₂₀ alkyl-O—Zn—O-heterocyclyl, heterocyclyl—O—Zn—O-heterocyclyl, and heterocyclyl—O—Zn—O-heteroaryl, wherein C₁₋₂₀ alkyl, heteroaryl, and heterocyclyl can be optionally substituted 1 to 3 times with R⁹;
R⁶ can be absent and, if present, is H or C₁₋₆ alkyl;
R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹¹, P(O)(OR¹⁰)(R¹¹)₂, P(O)(R¹¹)₂, BH₂,

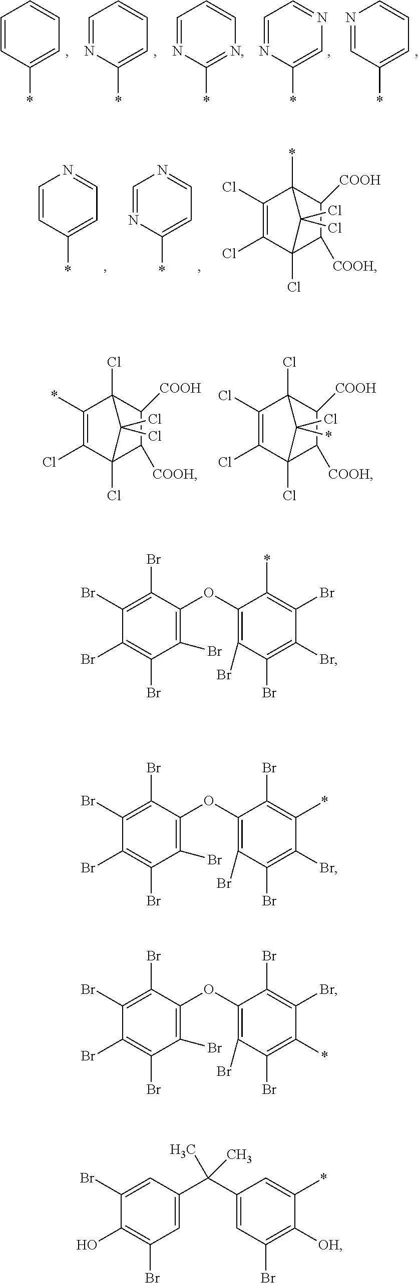

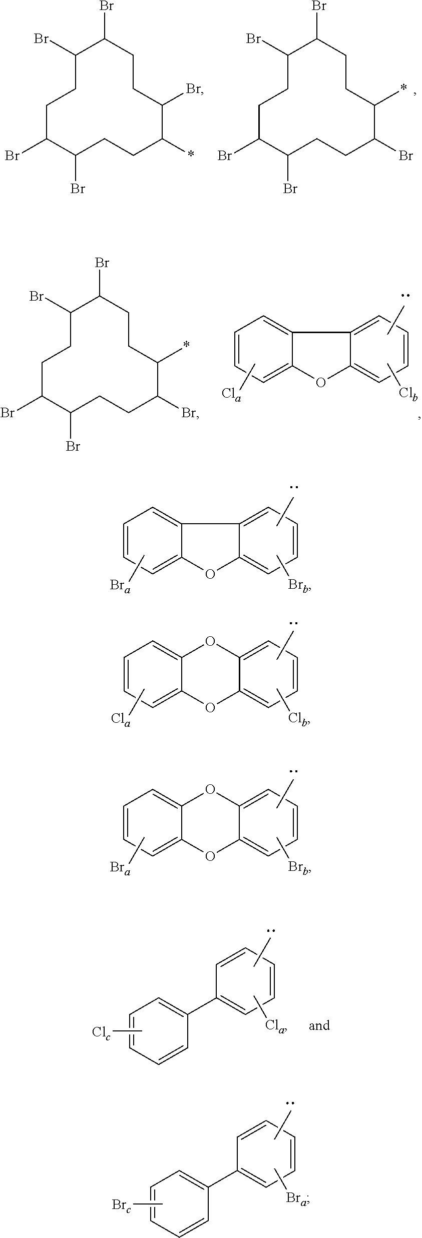

a is 1, 2, 3, or 4;
b is 1, 2, or 3;
c is 1, 2, 3, 4, or 5;
each R¹⁰ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl,
[symbol] is the point of attachment of R⁹ to R⁵;
i is 1 to 1,000,000;
j is 1 to 1,000,000;
m is 0 to 32; and
s is 0 to 32.

This process comprises:
providing the compound of Formula (IVb) described above;
providing a compound having the structure of Formula (XI):

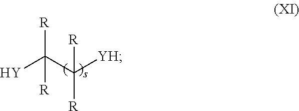

providing a compound having the structure of Formula (XII):

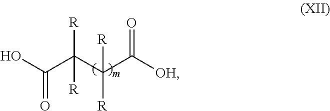

and reacting the compound of Formula (IVb), the compound of Formula (XI), and the compound of Formula (XII) under conditions effective to produce the polymer.

One aspect of the present application relates to a process of making a polymer of Formula (XVIII):

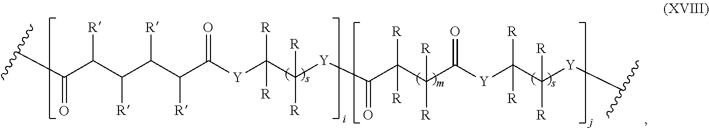

wherein
Y is NH or 0;
R is independently selected from the group consisting of H and C₁₋₂₀ alkyl;
each R′ is independently H or

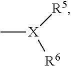

and only one of R′ is

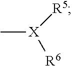

X is CH, O, S, N, P, or C═O;
R⁵ is selected from the group consisting of H, C₁₋₂₀ alkyl, heteroaryl, heterocyclyl, carborane, C₁₋₂₀ alkyl-O—Zn—O—C₁₋₂₀ alkyl, C₁₋₂₀ alkyl-O—Zn—O-heteroaryl, heteroaryl-O—Zn—O-heteroaryl, C₁₋₂₀ alkyl-O—Zn—O-heterocyclyl, heterocyclyl—O—Zn—O-heterocyclyl, and heterocyclyl—O—Zn—O-heteroaryl, wherein C₁₋₂₀ alkyl, heteroaryl, and heterocyclyl can be optionally substituted 1 to 3 times with R⁹;
R⁶ can be absent and, if present, is H or C₁₋₆ alkyl;
R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹¹, P(O)(OR¹⁰)(R¹¹)₂, P(O)(R¹¹)₂, BH₂,

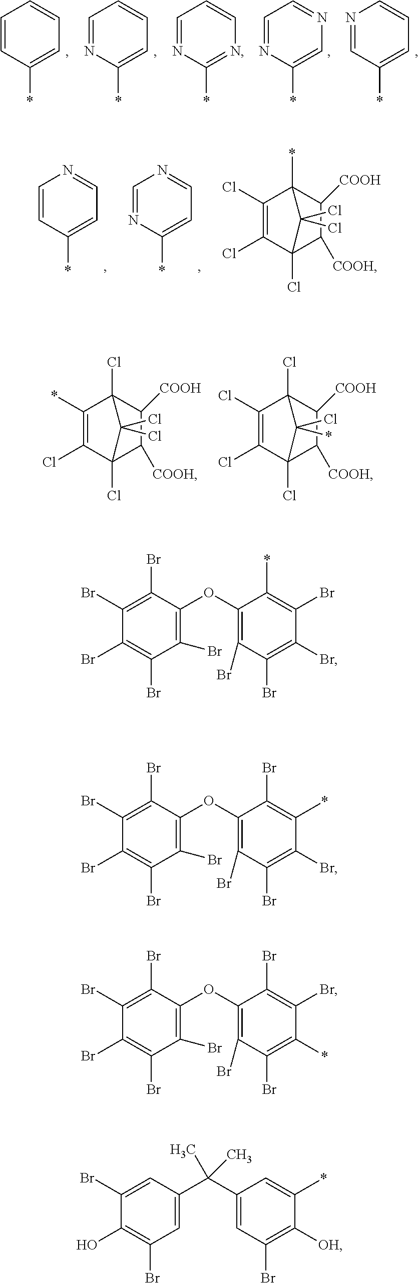

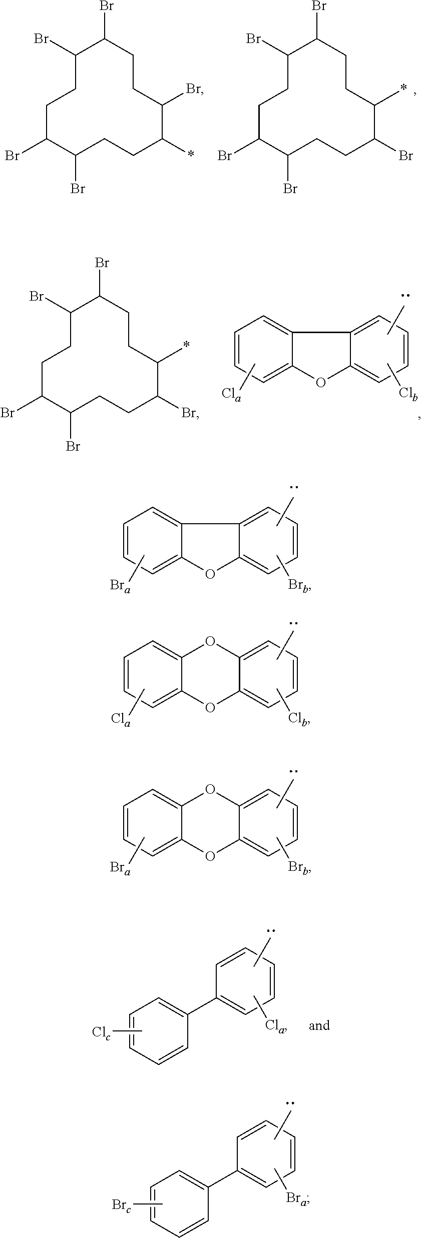

a is 1, 2, 3, or 4;
b is 1, 2, or 3;
c is 1, 2, 3, 4, or 5;
each R¹⁰ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl,
[symbol] is the point of attachment of R⁹ to R⁵;
i is 1 to 1,000,000;
j is 1 to 1,000,000;
m is 0 to 32;
s is 0 to 32; and
[symbol] is a terminal group of the polymer.

This process comprises:
providing the compound of Formula (IVb) described above;
providing a compound having the structure of Formula (XI):

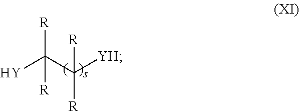

providing a compound having the structure of Formula (XII):

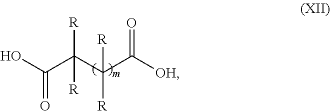

reacting the compound of Formula (IVb), the compound of Formula (XI), and the compound of Formula (XII) under conditions effective to produce the polymer of Formula (XVIII).

In one embodiment, the step of reacting the compound of formula (IVb), the compound of formula (XI), and the compound of formula (XII) comprises:
reacting the compound of formula (IVb) with the compound of formula (XI) to form a salt 1;
reacting the compound of formula (XII) with the compound of formula (XI) to form a salt 2; and
reacting the salt 1 with the salt 2 under conditions effective to produce the polymer.

In another embodiment, the step of reacting the salt 1 with the salt 2 comprises heating the salt 1 with the salt 2 under inert atmosphere in a reaction vessel. In one embodiment, the heating process is conducted under pressure.

During the process of making a polymer according to the present application, salt 1 and salt 2 can be used in any amount from 1 to 99%. In some embodiments, salt 1 and salt 2 are mixed at the ratio of 5% of salt 1 and 95% of salt 2, 10% of salt 1 and 90% of salt 2, 15% of salt 1 and 85% of salt 2, 20% of salt 1 and 80% of salt 2, 25% of salt 1 and 75% of salt 2, 30% of salt 1 and 70% of salt 2, 35% of salt 1 and 65% of salt 2, 40% of salt 1 and 60% of salt 2, 45% of salt 1 and 55% of salt 2, 50% of salt 1 and 50% of salt 2, 55% of salt 1 and 45% of salt 2, 60% of salt 1 and 40% of salt 2, 65% of salt 1 and 35% of salt 2, 70% of salt 1 and 30% of salt 2, 75% of salt 1 and 25% of salt 2, 80% of salt 1 and 20% of salt 2, 85% of salt 1 and 15% of salt 2, 90% of salt 1 and 10% of salt 2, or 95% of salt 1 and 5% of salt 2.

One aspect of the present application relates to a process of making a polymer of comprising a moiety:

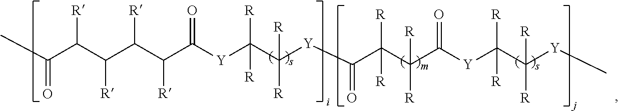

wherein
Y is NH or 0;
R is independently selected from the group consisting of H and C₁₋₂₀ alkyl;
each R′ is independently H or

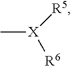

and only one of R′ is

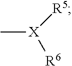

X is CH, O, S, N, P, or C═O;
R⁵ is selected from the group consisting of H, C₁₋₂₀ alkyl, heteroaryl, heterocyclyl, carborane, C₁₋₂₀ alkyl-O—Zn—O—C₁₋₂₀ alkyl, C₁₋₂₀ alkyl-O—Zn—O-heteroaryl, heteroaryl-O—Zn—O-heteroaryl, C₁₋₂₀ alkyl-O—Zn—O-heterocyclyl, heterocyclyl—O—Zn—O-heterocyclyl, and heterocyclyl—O—Zn—O-heteroaryl, wherein C₁₋₂₀ alkyl, heteroaryl, and heterocyclyl can be optionally substituted 1 to 3 times with R⁹;
R⁶ can be absent and, if present, is H or C₁₋₆ alkyl;
R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹¹, P(O)(OR¹⁰)(R¹¹)₂, P(O)(R¹)₂, BH₂,

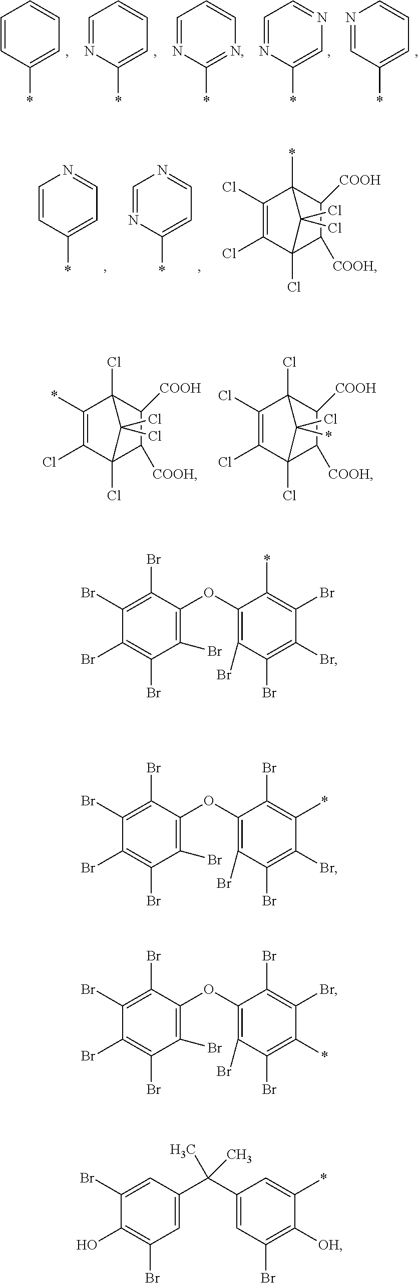

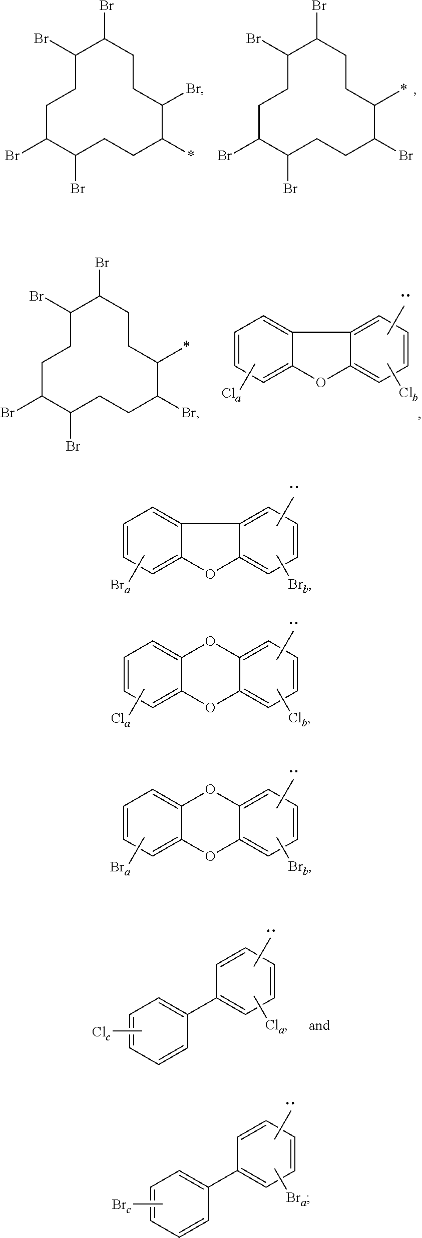

a is 1, 2, 3, or 4;
b is 1, 2, or 3;
cis 1,2,3,4, or 5;
each R¹⁰ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl,
[symbol] is the point of attachment of R⁹ to R⁵;
i is 1 to 1,000,000;
j is 1 to 1,000,000;
m is 0 to 32;
s is 0 to 32; and
[symbol] is a single bond attached to one of the positions shown.

This process comprises:
preparing the compound of Formula (IV) by the process described above;
providing a compound having the structure of Formula (XI):

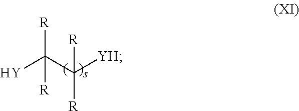

providing a compound having the structure of Formula (XII):

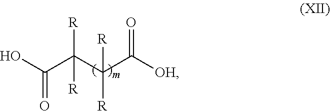

reacting the compound of Formula (IV), the compound of Formula (XI), and the compound of Formula (XII) under conditions effective to produce the polymer.

One aspect of the present application relates to a process of making a polymer of Formula (XIII):

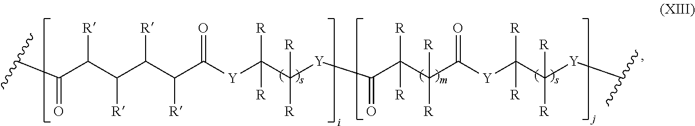

wherein
Y is NH or 0;
R is independently selected from the group consisting of H and C₁₋₂₀ alkyl;
each R′ is independently H or

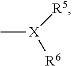

and only one of R′ is

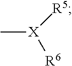

X is CH, O, S, N, P, or C═O;
R⁵ is selected from the group consisting of H, C₁₋₂₀ alkyl, heteroaryl, heterocyclyl, carborane, C₁₋₂₀ alkyl-O—Zn—O C₁₋₂₀ alkyl, C₁₋₂₀ alkyl-O—Zn—O-heteroaryl, heteroaryl-O—Zn—O-heteroaryl, C₁₋₂₀ alkyl-O—Zn—O-heterocyclyl, heterocyclyl—O—Zn—O-heterocyclyl, and heterocyclyl—O—Zn—O-heteroaryl, wherein C₁₋₂₀ alkyl, heteroaryl, and heterocyclyl can be optionally substituted 1 to 3 times with R⁹;
R⁶ can be absent and, if present, is H or C₁₋₆ alkyl;
R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹¹, P(O)(OR¹⁰)(R¹¹)₂, P(O)(R¹)₂, BH₂,

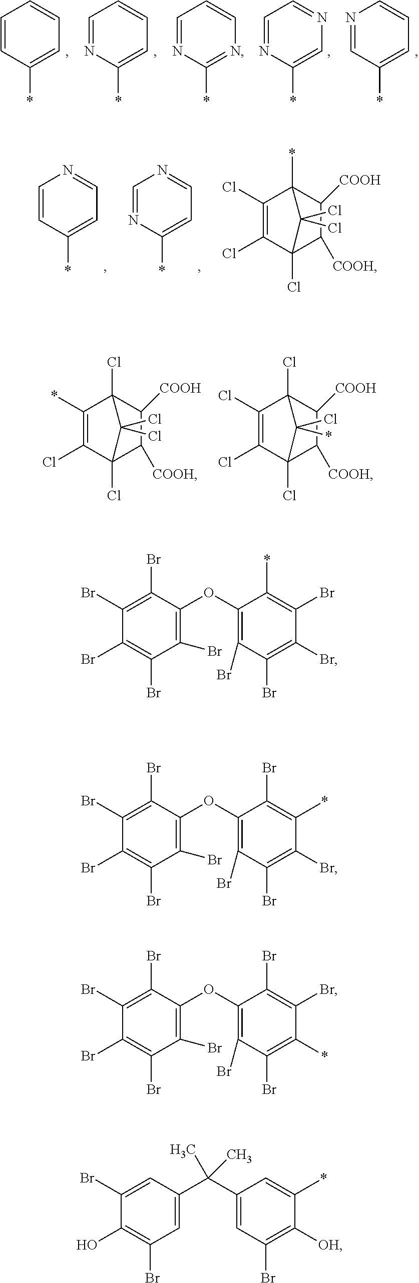

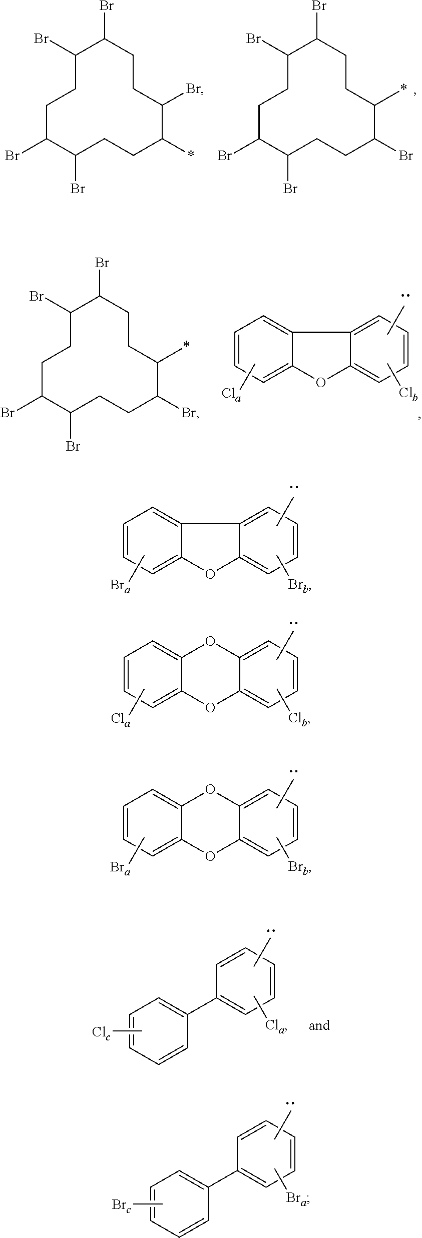

a is 1, 2, 3, or 4;
b is 1, 2, or 3;
c is 1,2,3,4, or 5;
each R¹⁰ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl,
[symbol] is the point of attachment of R⁹ to R⁵;
i is 1 to 1,000,000;
j is 1 to 1,000,000;
m is 0 to 32;
s is 0 to 32; and
[symbol] is a terminal group of the polymer;
[symbol] is a single bond attached to one of the positions shown.

This process comprises:
preparing the compound of Formula (IV) by the process described above;
providing a compound having the structure of Formula (XI):

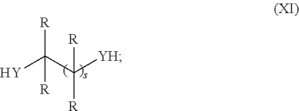

providing a compound having the structure of Formula (XII):

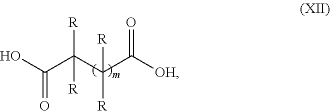

reacting the compound of Formula (IV), the compound of Formula (XI), and the compound of Formula (XII) under conditions effective to produce the polymer of Formula (XIII).

In one embodiment, the step of reacting the compound of formula (IV), the compound of formula (XI), and the compound of formula (XII) comprises:
reacting the compound of formula (IV) with the compound of formula (XI) to form a salt 1;
reacting the compound of formula (XII) with the compound of formula (XI) to form a salt 2; and
reacting the salt 1 with the salt 2 under conditions effective to produce the polymer.

In another embodiment, the step of reacting the salt 1 with the salt 2 comprises heating the salt 1 with the salt 2 under inert atmosphere in a reaction vessel. In one embodiment, the heating process is conducted under pressure.

During the process of making a polymer according to the present application, salt 1 and salt 2 can be used in any amount from 1 to 99%. In some embodiments, salt 1 and salt 2 are mixed at the ratio of 5% of salt 1 and 95% of salt 2, 10% of salt 1 and 90% of salt 2, 15% of salt 1 and 85% of salt 2, 20% of salt 1 and 80% of salt 2, 25% of salt 1 and 75% of salt 2, 30% of salt 1 and 70% of salt 2, 35% of salt 1 and 65% of salt 2, 40% of salt 1 and 60% of salt 2, 45% of salt 1 and 55% of salt 2, 50% of salt 1 and 50% of salt 2, 55% of salt 1 and 45% of salt 2, 60% of salt 1 and 40% of salt 2, 65% of salt 1 and 35% of salt 2, 70% of salt 1 and 30% of salt 2, 75% of salt 1 and 25% of salt 2, 80% of salt 1 and 20% of salt 2, 85% of salt 1 and 15% of salt 2, 90% of salt 1 and 10% of salt 2, or 95% of salt 1 and 5% of salt 2.

In another embodiment of the process of making a polymer of Formula (XVIII) or the polymer of Formula (XIII), the polymer of Formula (XVIII) or the polymer of Formula (XIII) has the structure of Formula (XIIIa):

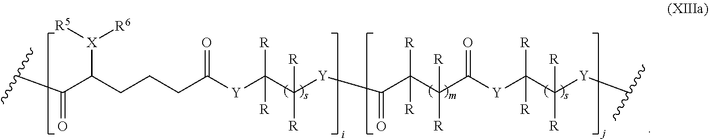

In another embodiment of the process of making a polymer of Formula (XVIII) or the polymer of Formula (XIII), the polymer of Formula (XVIII) or the polymer of Formula (XIII) has the structure of Formula (XIIIb):

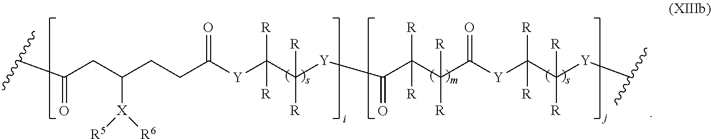

In another embodiment of the process of making a polymer of Formula (XVIII) or the polymer of Formula (XIII), the polymer of Formula (XVIII) or the polymer of Formula (XIII) has the structure of Formula (XIIIc):

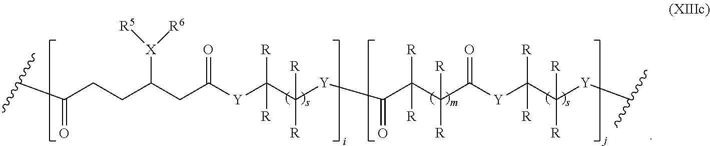

In another embodiment of the process of making a polymer of Formula (XVIII) or the polymer of Formula (XIII), the polymer of Formula (XVIII) or the polymer of Formula (XIII) has the structure of Formula (XIIId):

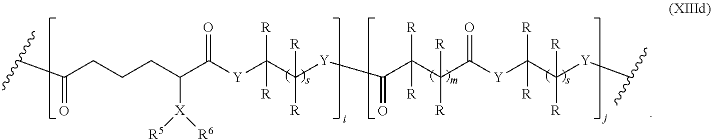

In another embodiment of the process of making a polymer of Formula (XVIII) or the polymer of Formula (XIII), the polymer of Formula (XVIII) or the polymer of Formula (XIII) has the structure of Formula (XIIIe):

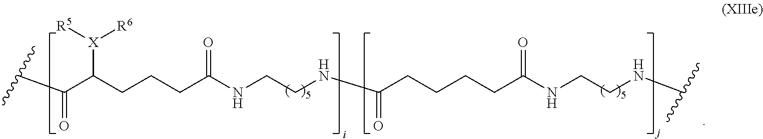

In another embodiment of the process of making a polymer of Formula (XVIII) or the polymer of Formula (XIII), the polymer of Formula (XVIII) or the polymer of Formula (XIII) has the structure of Formula (XIIIf):

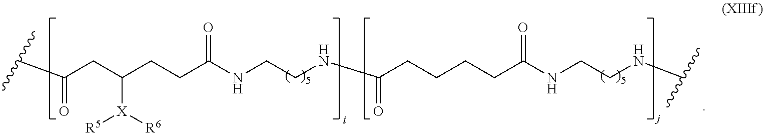

In another embodiment of the process of making a polymer of Formula (XVIII) or the polymer of Formula (XIII), the polymer of Formula (XVIII) or the polymer of Formula (XIII) has the structure of Formula (XIIIg):

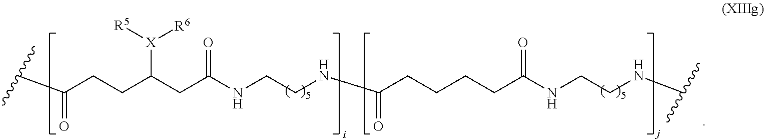

In another embodiment of the process of making a polymer of Formula (XVIII) or the polymer of Formula (XIII), the polymer of Formula (XVIII) or the polymer of Formula (XIII) has the structure of Formula (XIIIh):

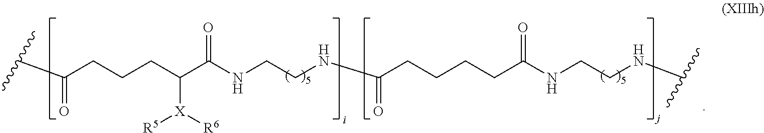

One aspect of the present application relates to a polymer comprising a moiety:

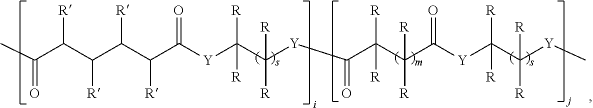

wherein
Y is NH or 0;
R is independently selected from the group consisting of H and C₁₋₂₀ alkyl;
each R′ is independently H or

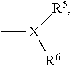

and only one of R′ is

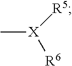

X is CH, O, S, N, P, or C═O;
R⁵ is selected from the group consisting of H, C₁₋₂₀ alkyl, heteroaryl, heterocyclyl, carborane, C₁₋₂₀ alkyl O—Zn—O C₁₋₂₀ alkyl, C₁₋₂₀ alkyl O—Zn—O heteroaryl, heteroaryl O—Zn—O heteroaryl, C₁₋₂₀ alkyl O—Zn—O heterocyclyl, heterocyclyl O—Zn—O heterocyclyl, and heterocyclyl O—Zn—O heteroaryl, wherein C₁₋₂₀ alkyl, heteroaryl, and heterocyclyl can be optionally substituted 1 to 3 times with R⁹;R⁶ can be absent and, if present, is H or C₁₋₆ alkyl;
R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹¹, P(O)(OR¹⁰)(R¹¹)₂, P(O)(R¹)₂, BH₂,

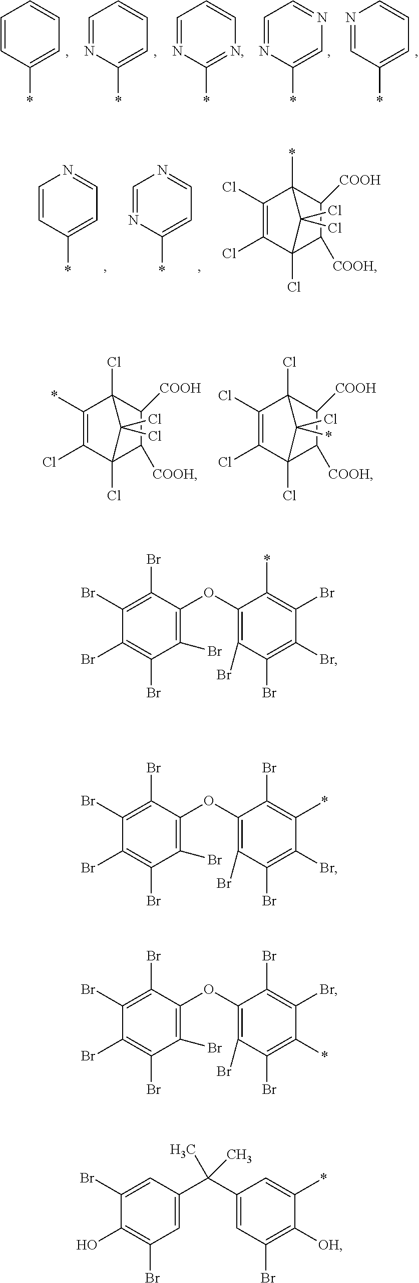

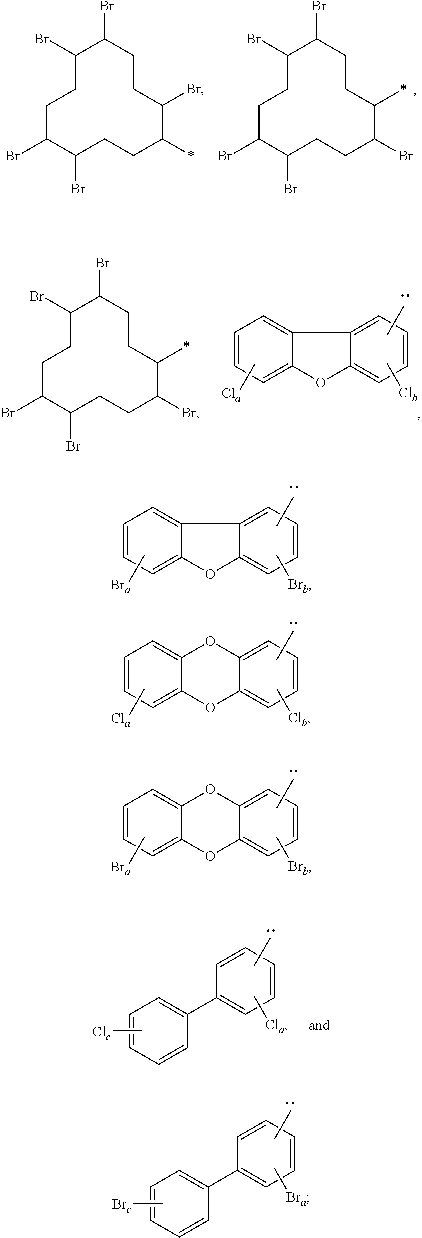

a is 1,2,3, or 4;
b is 1, 2, or 3;
c is 1, 2, 3, 4, or 5;
each R¹⁰ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl,
[symbol] is the point of attachment of R⁹ to R⁵;
i is 1 to 1,000,000;
j is 1 to 1,000,000;
m is 0 to 32;
s is 0 to 32; and
[symbol] is a single bond attached to one of the positions shown.

One aspect of the present application relates to a polymer of Formula (XIII):

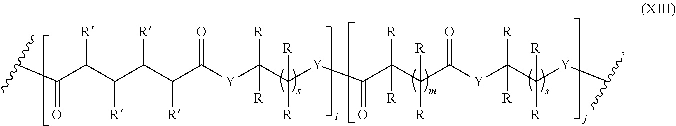

wherein
Y is NH or 0;
R is independently selected from the group consisting of H and C₁₋₂₀ alkyl;
each R′ is independently H or

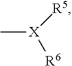

and only one of R′ is

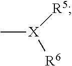

X is CH, O, S, N, P, or C═O;
R⁵ is selected from the group consisting of H, C₁₋₂₀ alkyl, heteroaryl, heterocyclyl, carborane, C₁₋₂₀ alkyl-O—Zn—O—C₁₋₂₀ alkyl, C₁₋₂₀ alkyl-O—Zn—O-heteroaryl, heteroaryl-O—Zn—O-heteroaryl, C₁₋₂₀ alkyl-O—Zn—O-heterocyclyl, heterocyclyl—O—Zn—O-heterocyclyl, and heterocyclyl—O—Zn—O-heteroaryl, wherein C₁₋₂₀ alkyl, heteroaryl, and heterocyclyl can be optionally substituted 1 to 3 times with R⁹;R⁶ can be absent and, if present, is H or C₁₋₆ alkyl;
R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹¹, P(O)(OR¹⁰)(R¹¹)₂, P(O)(R¹)₂, BH₂,

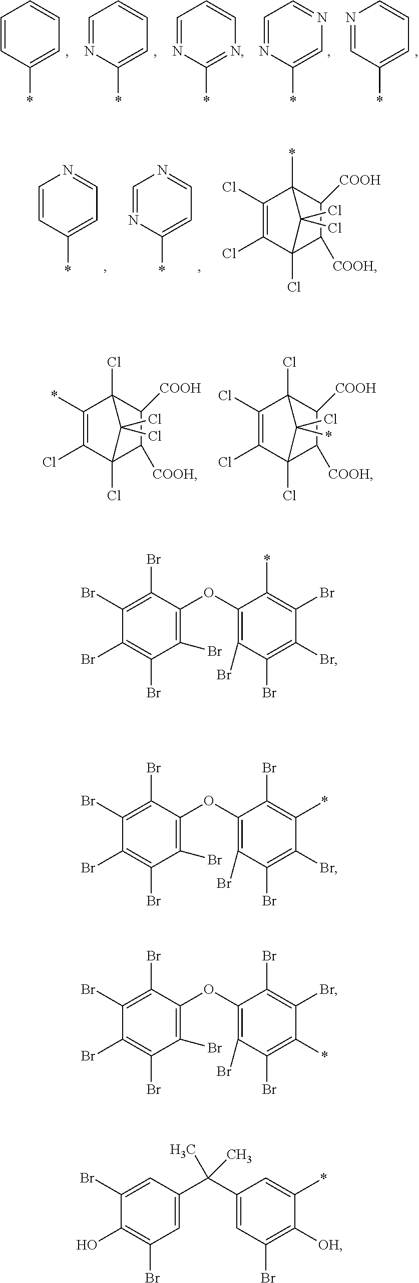

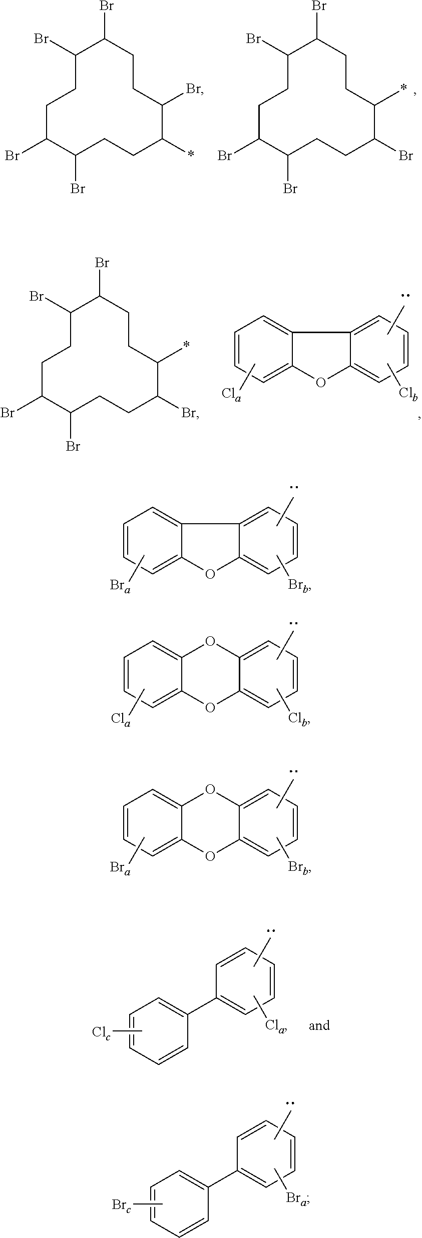

a is 1, 2, 3, or 4;
b is 1, 2, or 3;
c is 1,2,3,4, or 5;
each R¹⁰ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl,
[symbol] is the point of attachment of R⁹ to R⁵;
i is 1 to 1,000,000;
j is 1 to 1,000,000;
m is 0 to 32;
s is 0 to 32; and
[symbol] is a terminal group of the polymer;
[symbol] is a single bond attached to one of the positions shown.

In another embodiment of the polymer of Formula (XIII), the polymer has the structure of Formula (XIIIa), Formula (XIIIb), Formula (XIIIc), Formula (XIIId), Formula (XIIIe), Formula (XIIIf), Formula (XIIIg), and Formula (XIIIh).

In another embodiment of the polymer of Formula (XIII), the polymer is a statistical polymer.

In another embodiment of the polymer of Formula (XIII), the polymer is a random polymer.

In another embodiment of the polymer of Formula (XIII), the polymer is an alternating polymer.

In another embodiment of the polymer of Formula (XIII), the polymer is a block polymer.

One aspect of the present application relates to a process for preparation of a compound of Formula (XIV):

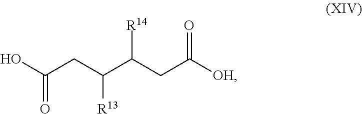

wherein
R¹³ is YR¹⁷R¹⁸;
R¹⁴ is ZR¹⁹R²⁰;
Y and Z are independently selected from CH, O, S, N, P, or C═O;
R¹⁷ and R¹⁹ are independently selected from the group consisting of H, C₁₋₂₀ alkyl, heteroaryl, heterocyclyl, carborane, C₁₋₂₀ alkyl-O—Zn—O C₁₋₂₀ alkyl, C₁₋₂₀ alkyl-O—Zn—O heteroaryl, heteroaryl-O—Zn—O-heteroaryl, C₁₋₂₀ alkyl-O—Zn—O-heterocyclyl, heterocyclyl O—Zn—O heterocyclyl, and heterocyclyl O—Zn—O heteroaryl, wherein C₁₋₂₀ alkyl, heteroaryl, and heterocyclyl can be independently optionally substituted 1 to 3 times with R²¹;
R¹⁸ and R²⁰ independently can be absent and, if present, are independently H or C₁₋₆ alkyl;
R²¹ is independently selected from the group consisting of H, C₁₋₆ alkyl, SH, NH₂, PH₂, P(O)(OR²²)₂, P(O)(OR²²)₃, P(O)(OR²²)₂R²³, P(O)(OR²²)(R²³)₂, P(O)(R²³)₂, BH₂,

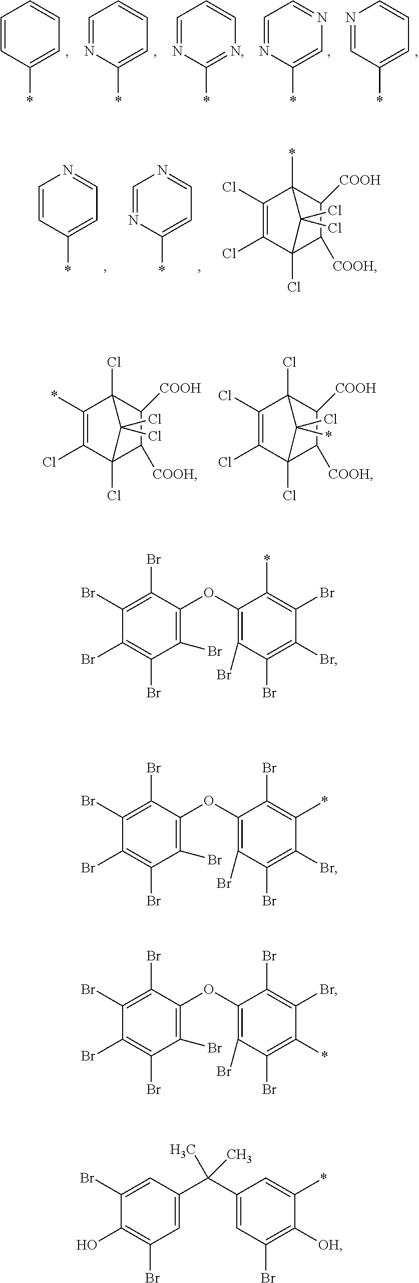

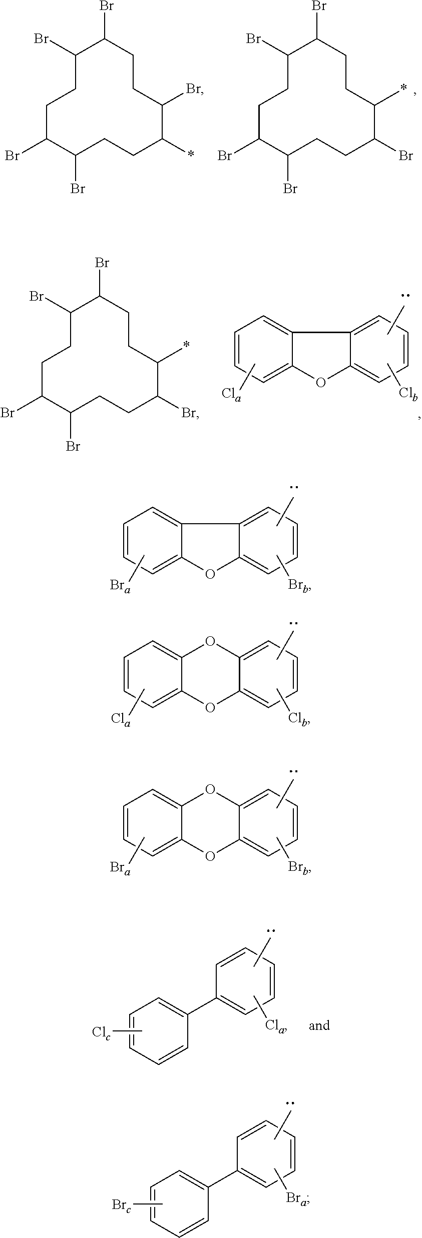

a is 1, 2, 3, or 4;
b is 1, 2, or 3;
c is 1,2,3,4, or 5;
each R²² is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R²³ is independently selected from the group consisting of H, C₁₋₆ alkyl, C₁₋₆ alkenyl, and aryl, wherein aryl can be optionally substituted 1 to 3 times with halogen, or C₁₋₆ alkyl,
[symbol] is the point of attachment of R²¹ to R¹⁷ or R¹⁹ or a salt thereof.

This process comprises:
providing a compound of Formula (XV) having the structure:

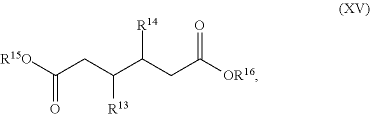

wherein
R¹⁵ is H or C₁₋₆ alkyl;
R¹⁶ is H or C₁₋₆ alkyl; and
forming the compound of Formula (I) from the compound of Formula (II).

In another embodiment, the process for preparation of a compound of Formula (XIV) comprises reacting the compound of Formula (XV) in the presence of a base to produce the compound of Formula (XVI).

In another embodiment of the process for preparation of a compound of Formula (XIV), the base is selected from the group consisting of alkali hydroxides, primary amines, secondary amines, and tertiary amines.

In another embodiment of the process for preparation of a compound of Formula (XIV), the base is selected from the group consisting of potassium hydroxide, sodium hydroxide, dimethyl formamide, 1,4-diazabicyclo[2.2.2]octene, triethylamine, 1,8-diazabicyclo[5.4.0]undec-7-ene, 1,5-diazabicyclo[4.3.0]non-5-ene 4-dimethylaminopyridine, 1-methylimidazole, and 1,1,3,3-tetramethylguanidine.

In another embodiment of the process for preparation of a compound of Formula (XIV), the step of providing a compound of Formula (XV) comprises providing a compound of Formula (XVI) having the structure:

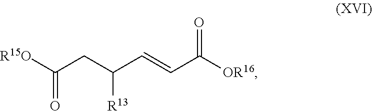

and forming the compound of Formula (XV) from the compound of Formula (XVI).

In another embodiment of the process for preparation of a compound of Formula (XIV), the step of forming the compound of Formula (XV) comprises reacting the compound of Formula (XVI) with a compound of Formula (XVII):

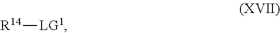

wherein LG¹ is a suitable leaving group.

In another embodiment of the process for preparation of a compound of Formula (XIV), the step of providing a compound of Formula (XVI) comprises providing a compound of Formula (XVIII) having the structure:

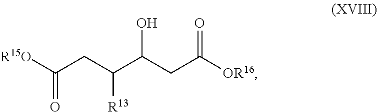

and forming the compound of Formula (XVI) from the compound of Formula (XVIII).

In another embodiment of the process for preparation of a compound of Formula (XIV), the step of forming the compound of Formula (XVI) comprises reacting the compound of Formula (XVIII) with a compound selected from the group consisting of 1,1,3,3-tetramethylguanidine (TMG), 1,5-Diazabicyclo[4.3.0]non-5-ene (DBN), 1,8-Diazabicyclo[5.4.0]undec-7-ene (DBU), triethylamine (TEA), tertiary amines, secondary amines, and primary amines.

In another embodiment of the process for preparation of a compound of Formula (XIV), the step of providing a compound of Formula (XVIII) comprises providing a compound of Formula (XIX) having the structure:

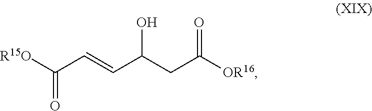

and forming the compound of Formula (XVIII) from the compound of Formula (XIX).

In another embodiment of the process for preparation of a compound of Formula (XIV), the step of forming the compound of Formula (XVIII) comprises reacting the compound of Formula (XIX) with a compound of Formula (XX):

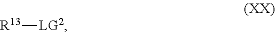

wherein LG² is a suitable leaving group.

In another embodiment of the process for preparation of a compound of Formula (XIV), the step of providing a compound of Formula (XIX) comprises providing a compound of Formula (XXI) having the structure:

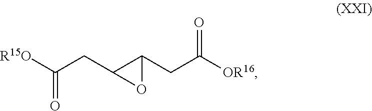

and forming the compound of Formula (XIX) from the compound of Formula (XXI).

In another embodiment of the process for preparation of a compound of Formula (XIV), the step of forming the compound of Formula (XIX) comprises reacting the compound of Formula (XXI) with compound selected from the group consisting of 1,1,3,3-tetramethylguanidine (TMG), 1,5-Diazabicyclo[4.3.0]non-5-leaving 1,8-Diazabicyclo[5.4.0]undec-7-ene (DBU), triethylamine (TEA), tertiary amines, secondary amines, and primary amines.

In another embodiment of the process for preparation of a compound of Formula (XIV), the step of providing a compound of Formula (XXI) comprises providing a compound of Formula (II) having the structure:

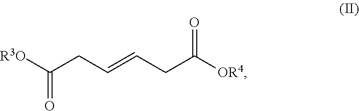

wherein
R³ is H, D, or C₁₋₆ alkyl;
R⁴ is H, D, or C₁₋₆ alkyl, and
forming the compound of Formula (XXI) from the compound of Formula (II).

In another embodiment of the process for preparation of a compound of Formula (XIV), the step of forming the compound of Formula (XXI) comprises reacting the compound of Formula (II) with a compound selected from the group consisting of meta-Chloroperoxybenzoic acid (mCPBA), peracetic acid, hydrogen peroxide, and peroxy acids.

According to any embodiment of the present application, i is from 1 to 1,000,000. For example, i is from 2 to 1,000,000, i is from 10 to 1,000,000, i is from 25 to 1,000,000, i is from 50 to 1,000,000, i is from 75 to 1,000,000, i is from 100 to 1,000,000, i is from 150 to 1,000,000, i is from 200 to 1,000,000, i is from 250 to 1,000,000, i is from 300 to 1,000,000, i is from 350 to 1,000,000, i is from 400 to 1,000,000, i is from 450 to 1,000,000, i is from 500 to 1,000,000, i is from 550 to 1,000,000, i is from 600 to 1,000,000, i is from 650 to 1,000,000, i is from 700 to 1,000,000, i is from 750 to 1,000,000, i is from 800 to 1,000,000, i is from 850 to 1,000,000, i is from 900 to 1,000,000, i is from 950 to 1,000,000, i is from 1,000 to 1,000,000, i is from 1,500 to 1,000,000, i is from 2,000 to 1,000,000, i is from 3,000 to 1,000,000, i is from 4,000 to 1,000,000, i is from 5,000 to 1,000,000, i is from 6,000 to 1,000,000, i is from 7,000 to 1,000,000, i is from 8,000 to 1,000,000, i is from 9,000 to 1,000,000, i is from 10,000 to 1,000,000, i is from 20,000 to 1,000,000, i is from 30,000 to 1,000,000, i is from 40,000 to 1,000,000, i is from 50,000 to 1,000,000, i is from 100,000 to 1,000,000, i is from 250,000 to 1,000,000, i is from 500,000 to 1,000,000, i is from 750,000 to 1,000,000. For example, i is from 2 to 850,000, i is from 10 to 700,000, i is from 50 to 600,000, i is from 100 to 500,000, i is from 250 to 500,000, i is from 500 to 500,000, i is from 1,000 to 500,000, i is from 2,000 to 500,000, i is from 10,000 to 500,000, i is from 100,000 to 500,000.

According to any embodiment of the present application, j is from 1 to 1,000,000. For example, j is from 2 to 1,000,000, j is from 10 to 1,000,000, j is from 25 to 1,000,000, j is from 50 to 1,000,000, j is from to 1,000,000, j is from 100 to 1,000,000, j is from 150 to 1,000,000, j is from 200 to 1,000,000, j is from 250 to 1,000,000, j is from 300 to 1,000,000, j is from 350 to 1,000,000, j is from 400 to 1,000,000, j is from 450 to 1,000,000, j is from 500 to 1,000,000, j is from 550 to 1,000,000, j is from 600 to 1,000,000, j is from 650 to 1,000,000, j is from 700 to 1,000,000, j is from 750 to 1,000,000, j is from 800 to 1,000,000, j is from 850 to 1,000,000, j is from 900 to 1,000,000, j is from 950 to 1,000,000, j is from 1,000 to 1,000,000, j is from 1,500 to 1,000,000, j is from 2,000 to 1,000,000, j is from 3,000 to 1,000,000, j is from 4,000 to 1,000,000, j is from 5,000 to 1,000,000, j is from 6,000 to 1,000,000, j is from 7,000 to 1,000,000, j is from 8,000 to 1,000,000, j is from 9,000 to 1,000,000, j is from 10,000 to 1,000,000, j is from 20,000 to 1,000,000, j is from 30,000 to 1,000,000, j is from 40,000 to 1,000,000, j is from 50,000 to 1,000,000, j is from 100,000 to 1,000,000, j is from 250,000 to 1,000,000, j is from 500,000 to 1,000,000, j is from 750,000 to 1,000,000. For example, j is from 2 to 850,000, j is from 10 to 700,000, j is from 50 to 600,000, j is from 100 to 500,000, j is from 250 to 500,000, j is from 500 to 500,000, j is from 1,000 to 500,000, j is from 2,000 to 500,000, j is from 10,000 to 500,000, j is from 100,000 to 500,000.

In one embodiment, i and j represent number average degrees of polymerization for repeat units of the polymer that are distributed throughout the polymer chain in a statistically defined manner.

According to any embodiment of the present application, the polymer can have a number average molecular weight (M) above 1 kDa, above 2 kDa, above 3 kDa, above 4 kDa, above 5 kDa, above 6 kDa, above 7 kDa, above 8 kDa, above 9 kDa, above 10 kDa, above 11 kDa, above 12 kDa, above 13 kDa, above 14 kDa, above 15 kDa, above 16 kDa, above 17 kDa, above 18 kDa, above 19 kDa, above 20 kDa, above 21 kDa, above 22 kDa, above 23 kDa, above 24 kDa, above 25 kDa, above 26 kDa, above 27 kDa, above 28 kDa, above 29 kDa, or above 30 kDa.

According to any embodiment of the present application, the polymer can have a number average molecular weight (M) ranging from 1 kDa to 200 kDa. For example, the polymer can have a number average molecular weight (M) from 1 kDa to 40 kDa, from 1 kDa to 35 kDa, from 2 kDa to 30 kDa, from 3 kDa to 30 kDa, from 4 kDa to 30 kDa, from 5 kDa to 30 kDa, from 6 kDa to 30 kDa, from 7 kDa to 30 kDa, from 8 kDa to 30 kDa, from 9 kDa to 30 kDa, from 10 kDa to 30 kDa, from 11 kDa to 30 kDa, from 12 kDa to 30 kDa, from 13 kDa to 30 kDa, from 14 kDa to 30 kDa, from 15 kDa to 30 kDa, from 2 kDa to 20 kDa, from 3 kDa to 20 kDa, from 4 kDa to 20 kDa, from 5 kDa to 20 kDa, from 6 kDa to 20 kDa, from 7 kDa to 20 kDa, from 8 kDa to 20 kDa, from 9 kDa to 20 kDa, from 10 kDa to 20 kDa, from 11 kDa to 20 kDa, from 12 kDa to 20 kDa, from 13 kDa to 20 kDa, from 14 kDa to 20 kDa, from 15 kDa to 20 kDa, from 2 kDa to 15 kDa, from 3 kDa to 15 kDa, from 4 kDa to 15 kDa, from 5 kDa to 15 kDa, from 6 kDa to 15 kDa, from 7 kDa to 15 kDa, from 8 kDa to 15 kDa, from 9 kDa to 15 kDa, from 10 kDa to 15 kDa, from 1 kDa to 10 kDa, from 2 kDa to 10 kDa, from 3 kDa to 10 kDa, from 4 kDa to 10 kDa, or from 5 kDa to 10 kDa.

The above disclosure is general. A more specific description is provided below in the following examples. The examples are described solely for the purpose of illustration and are not intended to limit the scope of the present application. Changes in form and substitution of equivalents are contemplated as circumstances suggest or render expedient. Although specific terms have been employed herein, such terms are intended in a descriptive sense and not for purposes of limitation.

### Examples

The following Examples are presented to illustrate various aspects of the present application, but are not intended to limit the scope of the claimed application.

### Example 1—Isomerization of trans-3-Hexenedioic Acid (t3HDA) to trans-2-Hexendioic Acid (t2HDA)

The isomerization of t3HDA to t2HDA was performed in aqueous media using potassium hydroxide (KOH) as a base catalyst. The effect of basicity was investigated by preparing solutions at 1, 5, and 10 M KOH. The tests were performed with t3HDA concentrations of 10 g/L and 80 g/L. 20 mL of the solutions was placed in scintillations vials and heated at 80° C. under vigorous stirring (400 rpm) using a magnetic stirring hot plate equipped with a reaction pie-block. 250 microliters aliquots of the solutions were taken at 0, 24, 48, and 72 hours. The aliquots were dried in air and the solid was dissolved in a deuterium oxide solution containing an internal standard (1 g/L of dimethyl malonic acid). The samples were then analyzed through nuclear magnetic resonance (¹H NMR) to quantify the isomers. The mass of the dried and deuterated samples was recorded before each analysis and all calculations were based on weight fractions.

Mechanistic studies to investigate the effect of pH were conducted by preparing t3HDA solutions at 10 g/L in deuterium oxide (D₂0) using also deuterated species to adjust the pH (1 M D₂SO₄, 1 M KOD, and 0.5 M of sodium phosphate buffer). The reaction was directly conducted in the NMR tubes by keeping an oil bath at 80° C. ¹H NMR analysis was then conducted at 0, 22, 44, and 91 hours. The tubes were quenched in ice prior to NMR analysis.

### Example 2—Isomerization of trans-3-Hexenedioate to trans-2-Hexenedioate

Dimethyl trans-3-hexenedioate (t3DMHDA) was purchased from TCI and used as received. The isomerization of t3DMHDA to dimethyl trans-2-hexenedioate (t2DMHDA) was performed under non-aqueous basic conditions to prevent hydrolysis of the ester functionalities while catalyzing the isomerization. Three methods were tested: in the first method, a 25 g/L solution of t3DMHDA was made in dimethyl formamide (DMF). In the second method, 50 mg of 1,4-diazabicyclo[2.2.2]octene (DABCO) was dissolved in 2 g of t3DMHDA, used here as the solvent. In the third method, t3DMHDA was dissolved in methanol to varying concentrations and 2-6 w/w % 1,1,3,3-tetramethylguanidine was used as a base catalyst. In the first two methods, the solutions were placed in scintillation vials, purged with argon, and heated at 120° C. for 72 hours resulting in 87% yield to t2DMHDA for the first method as verified with ¹H NMR. The second method proceeded to >99% conversion of t3DMHDA. However, a blackened solution indicated that there were side products which could not be verified with ¹H NMR. The third method resulted in a>60% yield to t2DMHDA and >90% t3DMHDA conversion within 12 hours at 65° C. using 5 w/w % 1,1,3,3-tetramethylguanidine.

### Example 3—Results of Examples 1 and 2

Isomerization of trans-3-Hexenedioic Acid to trans-2-Hexendioic Acid

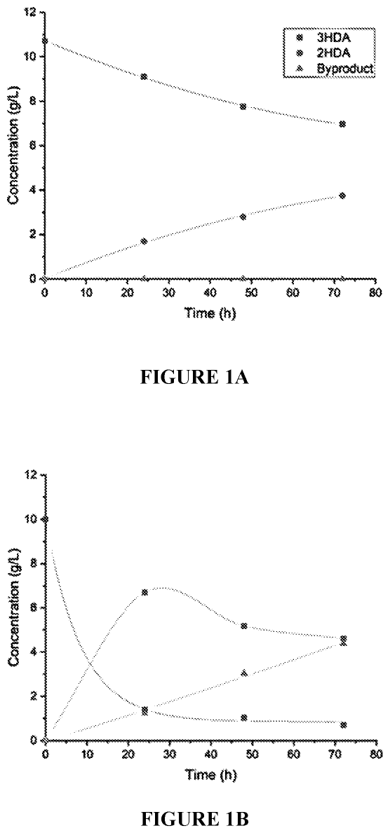

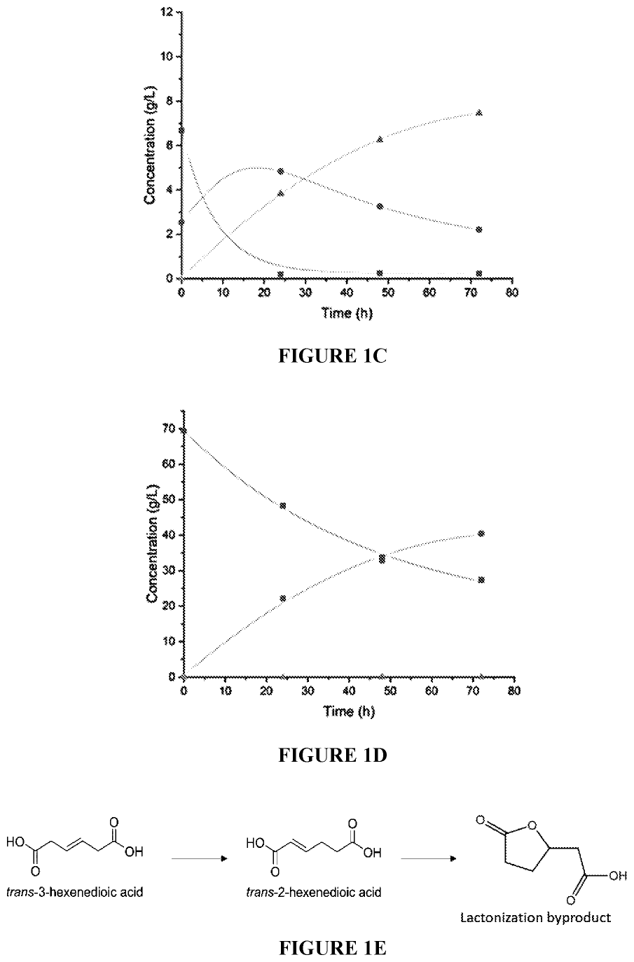

First, the effect of the concentration of the base catalyst on the performance of the reaction was analyzed. The isomerization, conducted at 80° C. and 10 g/L of t3HDA, proceeded with higher rates as the concentration of KOH is increased (FIGS. 1A-E). The best result was obtained when t3HDA was reacted with 5 M KOH; t2HDA reached 66% yield after 24 hours (FIG. 1i). It is worth noting that for 10 M KOH the reaction was so fast that at the initial time (when all the t3HDA was dissolved in solution) there was already some 2HDA present in the solution. It was also evident that by increasing the catalyst concentration a byproduct was formed. The compound was identified through NMR to be a lactonization byproduct of t2HDA (2-(5-oxotetrahydrofuran-2-yl)acetic acid). The kinetics of byproduct formation did not seem to be affected by the decrease in t3HDA concentration, which suggested that the lactone was formed by t2HDA isomerization. Additionally, the concentration profiles for t2HDA showed a maximum around 24 hours, the further decrease in t2HDA amount matches the lactone formed. A test was also conducted at 70 g/L of t3HDA and 1 M KOH (FIG. 1D). The higher concentration in t3HDA increased the reaction rate of the isomerization. However, in this case no byproduct was detected which indicated that the undesired reaction was also base catalyzed and occurred only at higher concentration of the base.

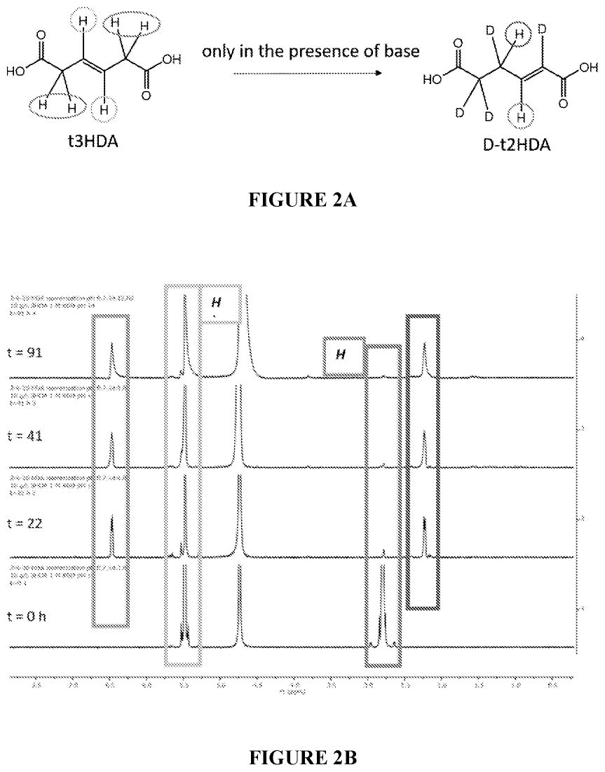

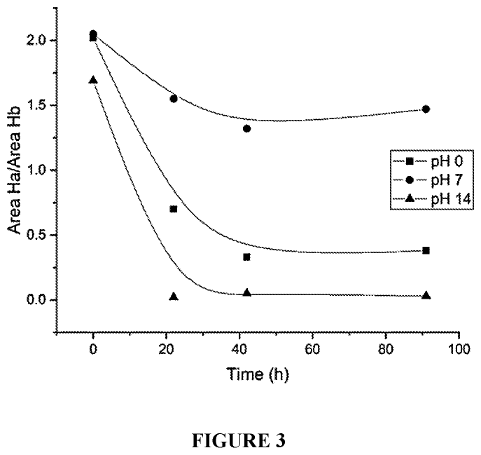

Mechanistic studies were conducted by using isotopically-labeled solvent. The experiments showed that the aliphatic protons (FIGS. 2A-B) of t3HDA were exchanged with deuterium under all of the tested reaction conditions, which indicated that t3HDA can be easily deprotonated to form a short-lived reactive intermediate. The evidence of this phenomenon was quantifiable by analyzing the ratio of the integrated peak area between the t3HDA protons as indicated in FIG. 2B. The overall trend of the ratio Ha/Hb decreased over time and after 24 hours it reached an equilibrium. Only at pH 0 and 14 there was a drastic decrease of the ratio, while for pH 7 the value remains around 1.5 (FIG. 3). In the presence of KOD (pH 14), the ratio reached approximately zero and the final isomerization from t3HDA to deuterated t2HDA occurred. This finding confirmed that the isomerization of t3HDA is base catalyzed.

In other words, the aliphatic protons of t3HDA are easily exchangeable by a base or an acid but only a strong base will fully extract a proton in the beta position. Based on the results of the exchanged deuterium/protons, the proposed mechanism sees an initial deprotonation of t3HDA aliphatic carbon and the formation of a carbanion (Scheme 1). The shift of the double bond from gamma to beta position then allows a more stable intermediate where the double bond is in resonance with the carboxylic functional group. The carbo-anion is protonated to form t2HDA while the catalyst is regenerated.

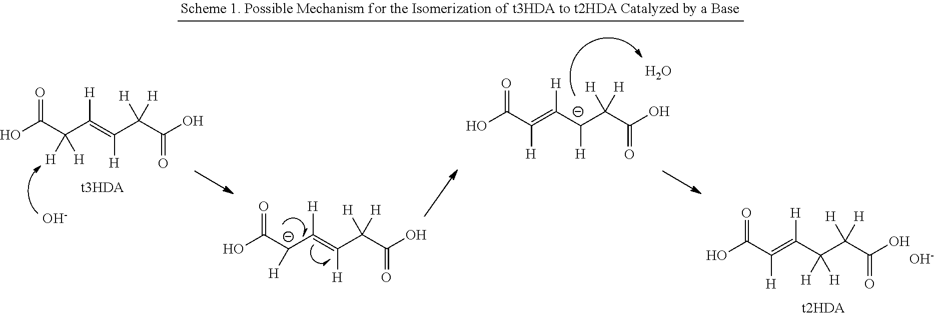

Isomerization of Dimethyl trans-3-Hexenedioate to Dimethyl trans-2-Hexenedioate

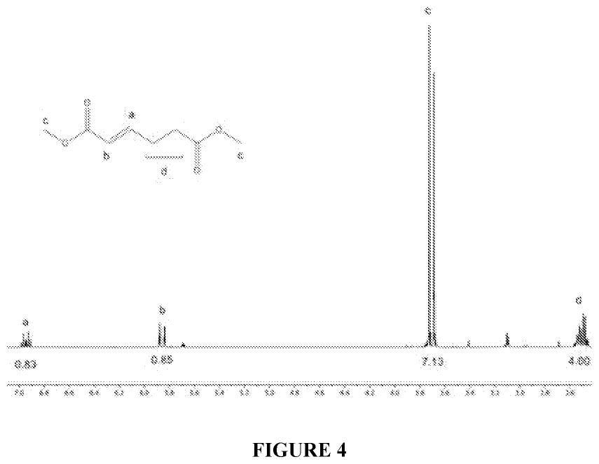

Methyl esters were able to participate in functionalization chemistry with a wider range of molecules than their carboxylic acid counterparts due to their better miscibility with and reactivity within organic solvents. To test the ability of amine solvents to catalyze this reaction, the isomerization of t3DMHDA was first conducted in DMF for 72 hours at 120° C., as shown by the ¹H NMR spectrum in FIG. 4.

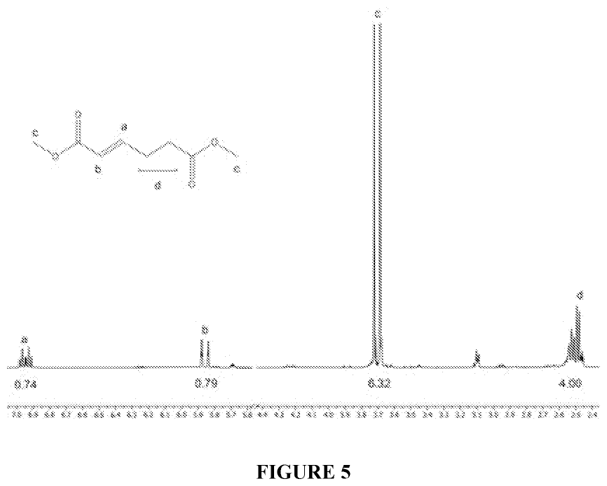

Yield was estimated as 87% with peak integration, however by modifying reaction time, temperature, and solvent this value could be increased. Another method was investigated which used t3DMHDA as the solvent while adding DABCO as a catalyst at 5 weight %, which resulted in production of the alpha-beta unsaturated ester after 72 hours at 120° C., as shown by ¹H NMR in FIG. 5.

Complete disappearance of the beta-gamma unsaturation hydrogens indicated>99% conversion of t3DMHDA. However, a blackened solution, which was not accounted for, was present after the reaction. Two possible explanations exist. It is possible that a highly exothermic isomerization could cause decomposition of DABCO even under inert conditions, which could generate a blackened char. However, a more likely explanation is the formation of amide between DABCO and the ester functionality of t3DMHDA. However, the reaction was promising since DABCO has been shown to be active in the Michael addition pathway, opening the possibility of “one pot” synthesis of functional monomers by conducting the previous reaction while adding thiols of interest.

### Example 4—Isomerization of trans-3-Hexenedioic Acid (t3HDA) to trans-2-Hexendioic Acid (t2HDA) with Improved Temperature Control

The isomerization of t3HDA to t2HDA was performed using a heating stirring plate equipped with a thermocouple inserted in a scintillation vial containing oil. This setup provided a better control of the reaction temperature than the setup used in Example 1. The reactions were performed in aqueous media using potassium hydroxide (KOH) as a base catalyst. The experiments were conducted at different temperatures and catalyst concentration to investigate the reaction rate, conversion, and yield. A volume of 20 mL of the solutions containing a 10_{gt3HDA}/L with 1 M or 10 M of KOH was put in a scintillations vial sealed with a septum cap. The vials were heated at 25, 50, 80, and 100° C. under vigorous stirring conditions (400 rpm). 250 microliters aliquots of the solutions were taken at 0, 1, 2, 3, 4, 24, 48, and 168 hours. The aliquots were quenched in ice and then dried in air. The remaining solid was then dissolved in a deuterium oxide solution containing an internal standard (1 g/L of dimethyl malonic acid). The samples were analyzed through nuclear magnetic resonance (¹H NMR) to quantify the isomers. The mass of the dried and deuterated samples was recorded before each analysis and all calculations were based on weight fractions.

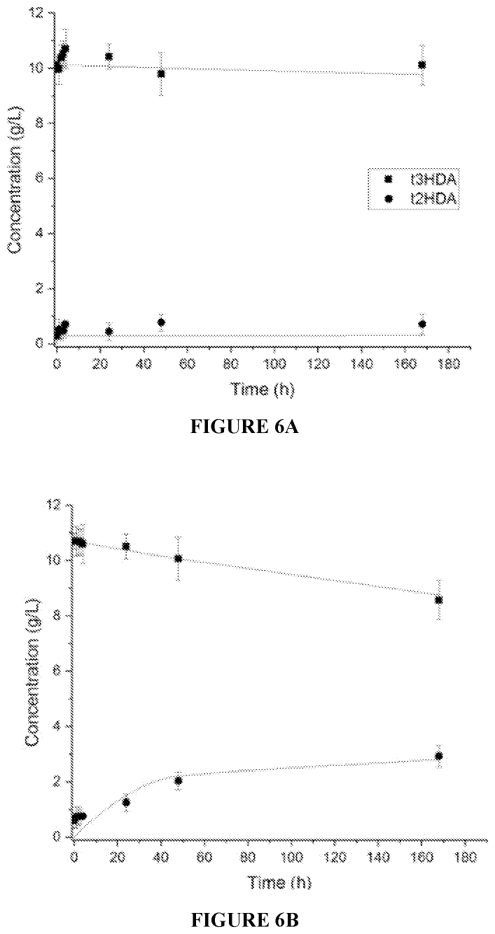

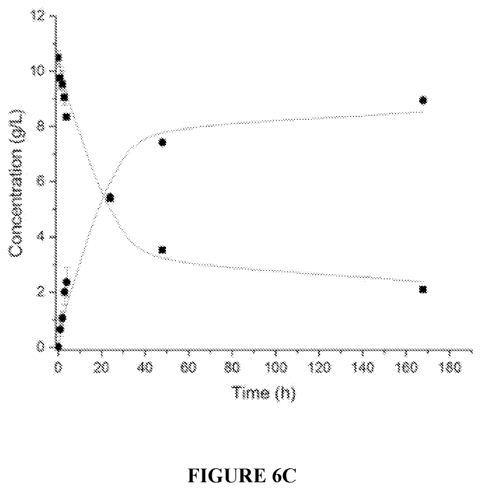

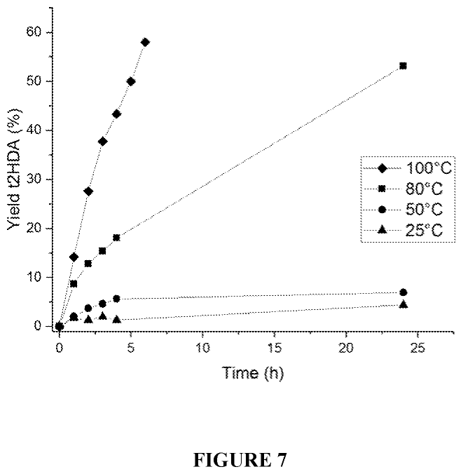

The reaction rate increased significantly with the temperature and after a certain amount of time an equilibrium was reached (FIG. 6A-C and FIG. 7). Interestingly, even at 25° C. there is formation of t2HDA, which suggests that the reaction is spontaneous. The highest yield obtained reached the value of 85% at 80° C. in 168 hours of reaction. However, high yields can also be reached in a short time by increasing the temperature further. At 100° C., the reaction reaches around 60% yield in the first 6 hours. This value was remarkably higher compared to the yield obtained for the other temperatures in the first hours of reaction (FIG. 7).

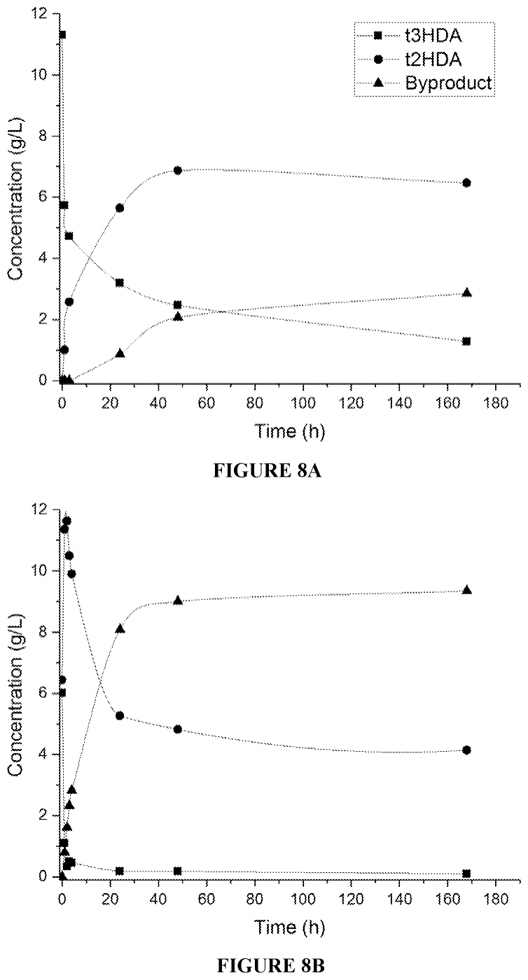

The concentration of the base catalyst has also an impact on the performance of the reaction. The isomerization of t3HDA proceeded with higher rates as the concentration of KOH is increased to 10 M (FIG. 8A-B). In the case of 10 M KOH, the reaction was so fast that at the initial time (when all the t3HDA was dissolved in solution) there was already some t2HDA present in the solution. It was also evident that by increasing the catalyst concentration a byproduct was formed. The compound was identified through NMR to be a lactonization byproduct of t2HDA (2-(5-oxotetrahydrofuran-2-yl)acetic acid). The kinetics of byproduct formation did not seem to be affected by the decrease in t3HDA concentration, which suggested that the lactone was formed by t2HDA isomerization. Additionally, the concentration profiles for t2HDA showed a maximum after 2 hours at 80° C. and at 48 hours for 25° C., the further decrease in t2HDA amount matches the lactone formed.

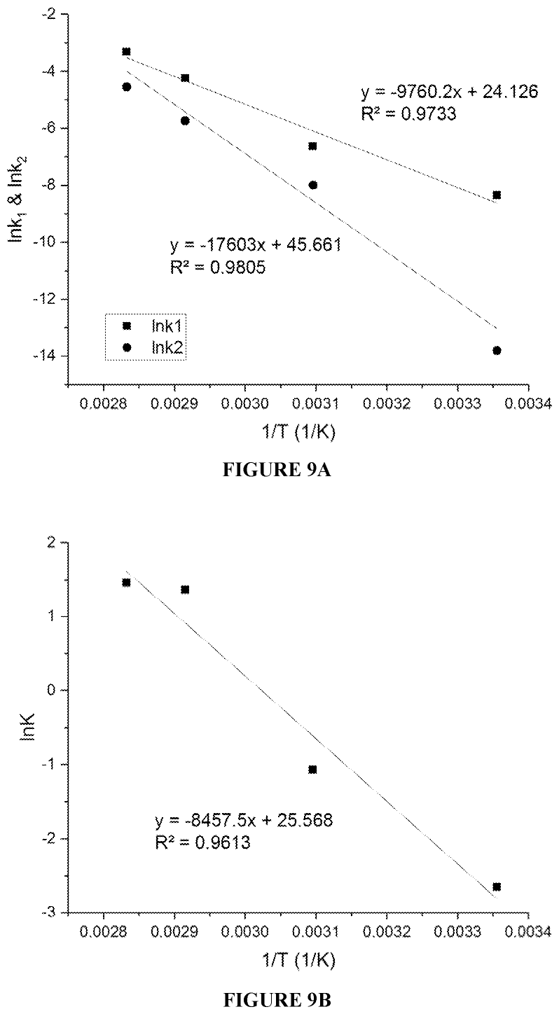

As it can be seen from the concentration profiles collected for solutions with 10 g t3HDA/L and 1 M of KOH, the reaction reaches an equilibrium for all of the investigated temperatures. The constant corresponding to the equilibrium between t3HDA and t2HDA is temperature-dependent, as can be seen in FIG. 9A. The evaluation of the equilibrium constant at different temperatures allowed to also extract some important thermodynamic quantities, which are presented in the Table 1 below. The reaction is endothermic and spontaneous as the value of enthalpy and entropy are both positive, which confirms the spontaneous formation of t2HDA even at the very beginning of the reaction.

TABLE 1
Thermodynamic and Kinetic Parameters

| | | |
|---|---|---|
| Ea1 | 81.10 | kJ/mol |
| Ea2 | 146.28 | kJ/mol |
| ΔH | 70.27 | kJ/mol |
| ΔS | 0.212 | kJ/mol*K |
| k01 | 2.58E+11 | 1/s |
| k02 | 4.12E+17 | 1/s |

Based on these results, a simple rate law was hypothesized, and the kinetic constants were obtained by fitting the concentration profiles at different temperatures. The rate constants were used to draw an Arrhenius plot which allows the extraction of activation energies and pre-exponential factors for the reaction kinetics (FIG. 9B).

### Example 5—Isomerization of Dimethyl-3-Hexenedioate and Subsequent Functionalization with DOPO

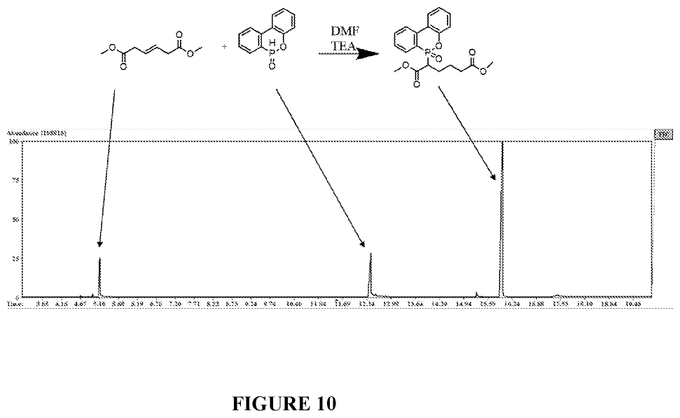

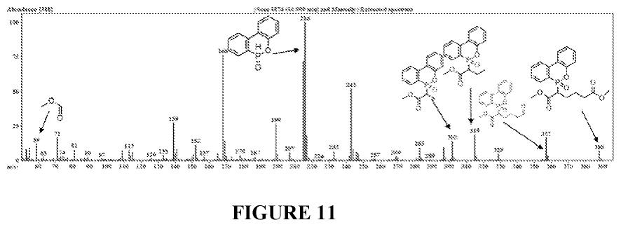

Reaction between 0.2 g of dimethyl-3-hexenedioate and 0.25 g of dried DOPO was carried out with 1:1 (v/v) of dimethylformamide and triethylamine at 85° C. for 5 days. The GC trace after 5 days showed the formation of desired product (FIG. 10). Fragmentation analysis of the product peak is shown in FIG. 11.

### Example 6—Isomerization of trans-3-Hexenedioic Acid, Functionalization with Aromatic Thiol, and Synthesis of the Corresponding Salt

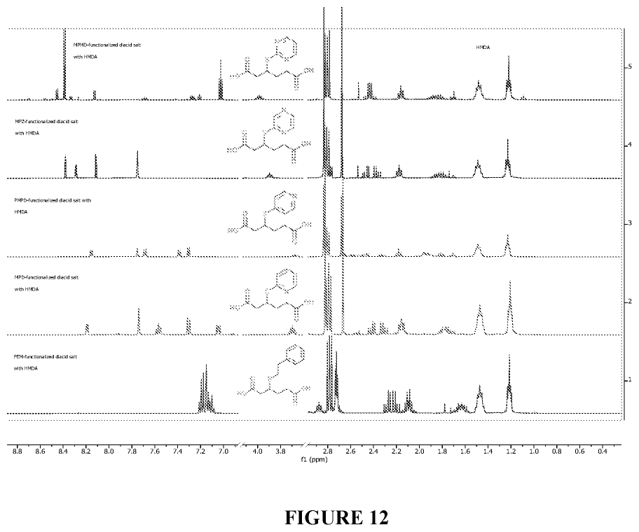

A series of aromatic thiols were used to functionalize trans-3-hexenedioic acid (t3HDA) by first undergoing isomerization to trans-2-hexenedioic acid (t2HDA). In a beaker, a mixture of 5.0 milliliters of dimethyl formamide (DMF) and 5.0 milliliters of triethylamine (TEA) were added to 1.0 grams of t3HDA and 2.0 grams of each corresponding thiol. The thiols used were phenyl ethyl mercaptan (PEM), 2-mercaptopyridine (MPD), 4-mercaptopyridine (PMPD), mercaptopyrazine (MPZ), and mercaptopyrimidine (MPMD). The solutions were poured into vials, the atmosphere was purged with nitrogen and the vials heated to 80° C. and kept at this temperature for 96 hours. During the reaction, t3HDA underwent a base catalyzed isomerization to t2HDA and subsequent base catalyzed 1,4 addition of the thiol to form the functionalized diacid. The vials were then poured into beakers and each solution was diluted with 90 milliliters of DMF. In a separate beaker, a solution of 1.65 grams of hexamethylenediamine (HMDA) was prepared in 100 milliliters of DMF for each reaction. The solutions of HMDA and diacid were combined in a beaker and left to stir, then the precipitate was collected. The precipitate, which typically presented as a yellow powder, was washed with tetrahydrofuran and put under reduced pressure at 80° C. to remove residual solvent. The resulting salts had ¹H NMR spectra as shown in FIG. 12.

### Example 7—Synthesis of Aromatic Thiol Functionalized Nylon 66

In a typical nylon 66 polycondensation with the hexamethylenediammonium adipate (nylon) salt and thiol-functionalized salt synthesized using the method described in Example 5 was used to yield 25% incorporation of functionalized monomer with respect to adipic acid. These are referred to as 25% functionalized bioadvantaged nylon (BAN25) salts, and the resulting polymers are called BAN25 polymers. BAN50 polymers were also produced by using a 1:1 ratio of nylon salt to thiol functionalized salt, however differential scanning calorimetry (DSC) showed these polymers were amorphous.

To produce the BAN25 salts, 100 mg of thiol-functionalized salt was combined with 211 mg of nylon salt, except in the case of PEM functionalized salt where 197 mg of nylon salt was used. To produce. The BAN25 salts were then placed in an aluminum pan and added to a quartz glass tube. The tube was then added to a tube furnace and purged with Argon gas for 10 minutes, heated to 260° C., and kept at this temperature for 30 minutes under a continuous Argon gas flow of 15 mL/min. The resulting polymers were collected and the following gel permeation chromatography (GPC), microcalorimetry (MCC), thermogravimetric analysis (TGA), and DSC data were obtained (Table 2).

TABLE 2
Polymer and Flame Retardance Properties: Molecular Weight (Mₙ) and Dispersity (Ð) in
Terms of PMMA from GPC, Onset Temperature (Tₒₙₛₑₜ), and Peak Weight Loss Temperature (T_{pwl})
from TGA, Percent Crystallinity (χ_{c}) from DSC, Peak Heat Release Rate (PHRR) and
Total Heat Release (THR) from MCC, and Char Residue at 600° C. (Char) from TGA

| | Mₙ | | | T_{pwl} | χ_{c} | PHRR | | |
|---|---|---|---|---|---|---|---|---|
| Polymer | (kDA) | Ð | Tₒₙₛₑₜ (° C.) | (° C.) | (%) | (W/g) | THR (kJ/g) | Char (%) |
| Nylon | 3.2 | 2.4 | 411.3 | 443.4 | 27.4 | 587 ± 21 | 28.17 ± 0.05 | 3.44 ± 0.77 |
| 66 | | | | | | | | |
| BAN25- | 9.1 | 4.8 | 399.6 | 440.1 | 11.3 | 590 ± 18 | 28.80 ± 0.14 | 6.70 ± 0.57 |
| PEM | | | | | | | | |
| BAN25- | 2.3 | 2.6 | 407.4 | 445.2 | 13.4 | 613 ± 18 | 28.53 ± 0.13 | 6.79 ± 0.46 |
| MPD | | | | | | | | |
| BAN25- | 3.0 | 2.6 | 398.9 | 436.9 | 14.6 | 634 ± 9 | 28.63 ± 0.17 | 8.53 ± 0.52 |
| PMPD | | | | | | | | |
| BAN25- | 4.2 | 3.1 | 409.5 | 447.1 | 11.4 | 889 ± 21 | 27.43 ± 0.13 | 11.13 ± 0.60 |
| MPZ | | | | | | | | |
| BAN25- | 3.6 | 3.7 | 370.1 | 383.4 | 12.5 | 580 ± 9 | 29.17 ± 0.19 | 8.39 ± 0.75 |
| MPMD | | | | | | | | |

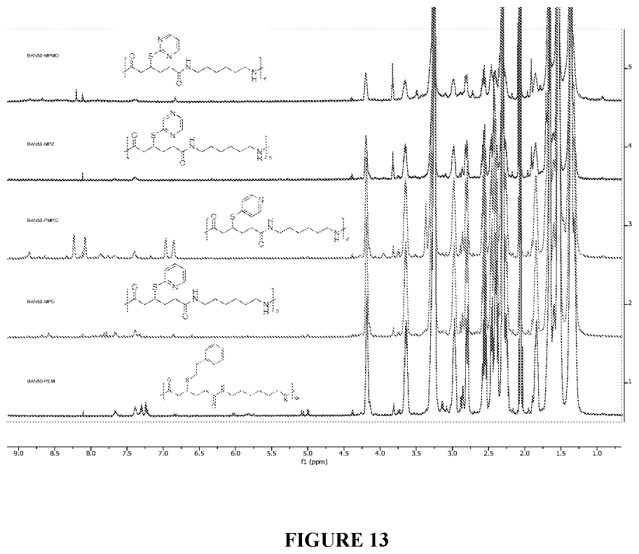

To produce the BAN50 salts, 200 mg of thiol-functionalized salt was combined with 141 mg of nylon salt, except in the case of PEM functionalized salt where 132 mg of nylon salt was used. The BAN50 salts were placed in an aluminum pan and inserted into a quartz glass tube. The tube was then placed inside a tube furnace and purged with Argon gas for 10 minutes, then heated to 260° C. and kept at this temperature for 30 minutes under Argon gas flow of 15 mL/min. The resulting polymers were then collected and the following ¹H NMR spectra were obtained (FIG. 13).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A process for preparation of a compound of Formula (I): 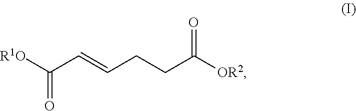
wherein
R¹ is H, D, or C₁₋₆ alkyl;
R² is H, D, or C₁₋₆ alkyl,
or a salt thereof, said
process comprises:
providing a compound of Formula(II) having the structure: 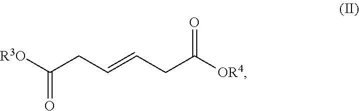
wherein
R³ is H, D, or C₁₋₆ alkyl;
R⁴ is H, D, or C₁₋₆ alkyl, and
forming the compound of Formula (I) from the compound of Formula (II).

2-13. (canceled)

14. A process for preparation of a compound of Formula (IV): 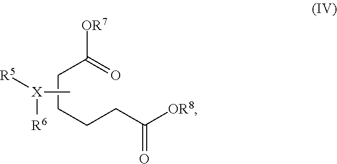
wherein
X is CH, O, S, N, P, or C═O;
R⁵ is selected from the group consisting of H, C₁₋₂₀ alkyl, heteroaryl, heterocyclyl, carborane, C₁₋₂₀ alkyl O—Zn—O C₁₋₂₀ alkyl, C₁₋₂₀ alkyl O—Zn—O heteroaryl, heteroaryl O—Zn—O-heteroaryl, C₁₋₂₀ alkyl-O—Zn—O-heterocyclyl, heterocyclyl—O—Zn—O-heterocyclyl, and heterocyclyl—O—Zn—O-heteroaryl, wherein C₁₋₂₀ alkyl, heteroaryl, and heterocyclyl can be optionally substituted 1 to 3 times with R⁹;
R⁶ can be absent and, if present, is H or C₁₋₆ alkyl;
R⁷ is H or C₁₋₆ alkyl;
R⁸ is H or C₁₋₆ alkyl;
R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹¹, P(O)(OR¹⁰)(R¹¹)₂P(O)(R¹¹)₂ BH₂, 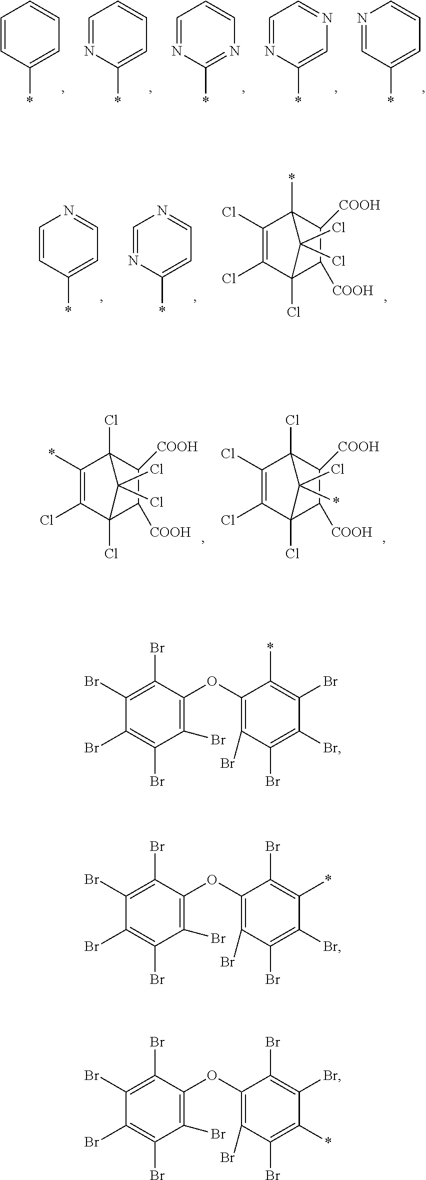 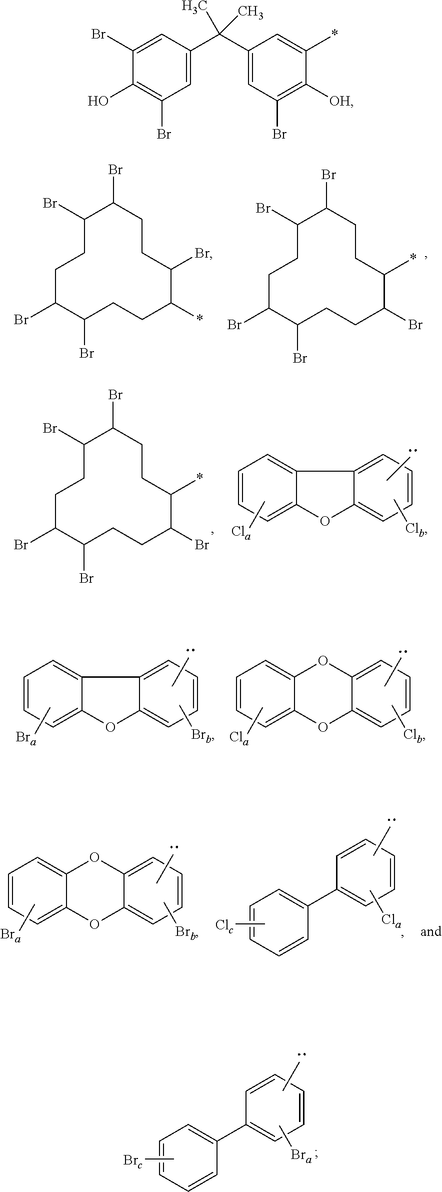
a is 1, 2, 3, or 4;
b is 1, 2, or 3;
c is 1, 2, 3, 4, or 5;
each R¹⁰ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl, [symbol]
is the point of attachment of R⁹ to R⁵;
or a salt thereof, said
process comprises:
providing a compound of Formula (I) prepared according to the process of claim 1, and
forming the compound of Formula (IV) from the compound of Formula (I).

15-25. (canceled)

26. A process for preparation of the compound claim 40
process comprises:
providing a compound of Formula (VII) having the structure: 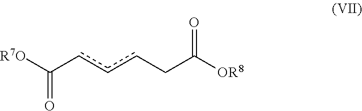
and forming the compound of Formula (IVb) from the compound of Formula (VII).

27-39. (canceled)

40. A compound of Formula (IVb): 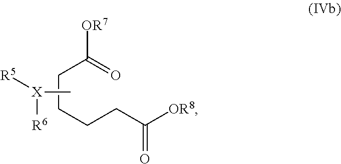
wherein
X is CH, O, S, N, P, or C═O;
R⁵ is selected from the group consisting of H, C₁₋₂₀ alkyl, heteroaryl, heterocyclyl, carborane, C₁₋₂₀ alkyl-O—Zn—O—C₁₋₂₀ alkyl, C₁₋₂₀ alkyl-O—Zn—O-heteroaryl, heteroaryl-O—Zn—O-heteroaryl, C₁₋₂₀ alkyl-O—Zn—O-heterocyclyl, heterocyclyl—O—Zn—O-heterocyclyl, and heterocyclyl—O—Zn—O-heteroaryl, wherein C₁₋₂₀ alkyl, heteroaryl, and heterocyclyl can be optionally substituted 1 to 3 times with R⁹;
R⁶ can be absent and, if present, is H or C₁₋₆ alkyl;
R⁷ is H or C₁₋₆ alkyl;
R⁸ is H or C₁₋₆ alkyl;
R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹, P(O)(OR¹⁰)(R¹¹)₂ P(O)(R¹¹)₂ BH₂, 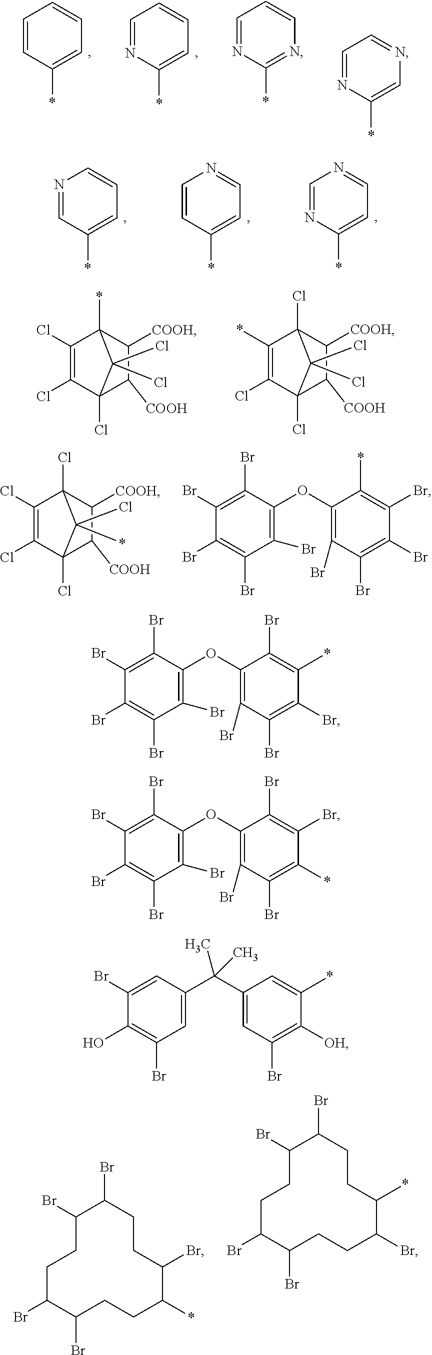 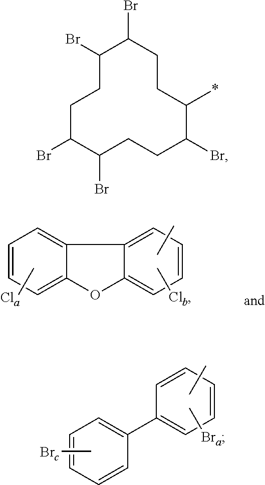
a is 1, 2, 3, or 4;
b is 1, 2, or 3;
c is 1, 2, 3, 4, or 5;
each R¹⁰ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl, [symbol]
is the point of attachment of R⁹ to R⁵;
with the proviso that if R⁷ is H, then R⁸ is not H,
or a salt thereof.

41. A process of making a polymer of Formula (X): 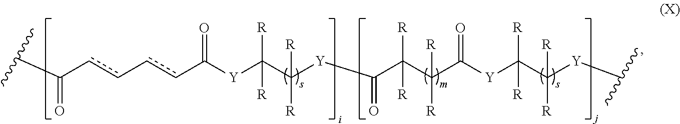
wherein
Y is NH or O;
R is independently selected from the group consisting of H and C₁₋₂₀ alkyl;
i is 1 to 1,000,000;
j is 1 to 1,000,000;
m is 0 to 32;
s is 0 to 32; and [symbol]
is a terminal group of the polymer; [symbol]
is a single or a double bond, and only one of [symbol] is a double bond; said process comprises:
preparing the compound of Formula (I) by the process of claim 1;
providing a compound having the structure of Formula (XI): 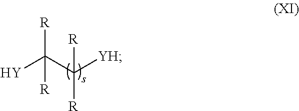
providing a compound having the structure of Formula (XII): 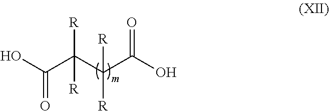
reacting the compound of Formula (I), the compound of Formula (XI), and the compound of Formula (XII) under conditions effective to produce the polymer of Formula (X).

42-45. (canceled)

46. A polymer of Formula (X): 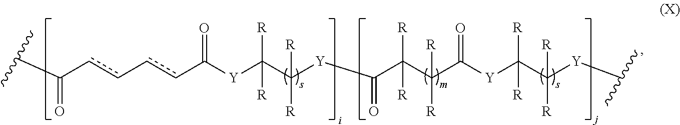
wherein
Y is NH or O;
R is independently selected from the group consisting of H and C₁₋₂₀ alkyl;
i is 1 to 1,000,000;
j is 1 to 1,000,000;
m is 0 to 32;
s is 0 to 32; and [symbol]
is a terminal group of the polymer; [symbol]
is a single or a double bond, and only one of [symbol] is a double bond.

47. The polymer of claim 46, wherein the polymer has the structure of Formula (Xa):

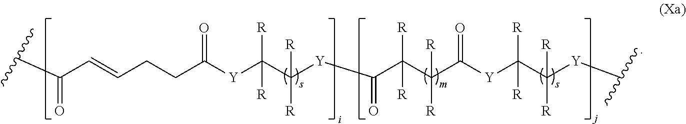

48. The polymer of claim 46, wherein the polymer has the structure of Formula (Xb):

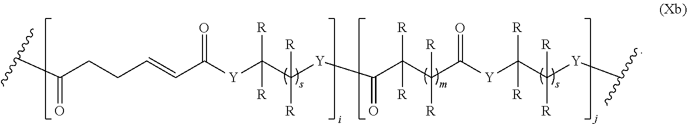

49. The polymer of claim 46, wherein the polymer has the structure of Formula (Xc):

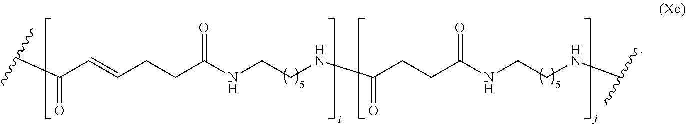

50. The polymer of claim 46, wherein the polymer has the structure of Formula (Xd):

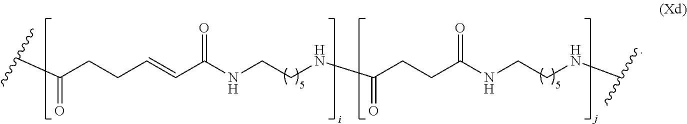

51. The polymer according to claim 46, wherein the polymer is a statistical polymer.

52. The polymer according to claim 46, wherein the polymer is a random polymer.

53. The polymer according to claim 46, wherein the polymer is an alternating polymer.

54. The polymer according to claim 46, wherein the polymer is a block polymer.

55. A process of making a polymer of Formula (XVIII): 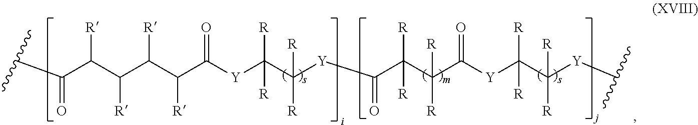
wherein
Y is NH or O;
R is independently selected from the group consisting of H and C₁₋₂₀ alkyl;
each R′ is independently H or 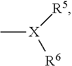
and only one of R′ is 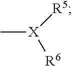
X is CH, O, S, N, P, or C═O;
R⁵ is selected from the group consisting of H, C₁₋₂₀ alkyl, heteroaryl, heterocyclyl, carborane, C₁₋₂₀ alkyl-O—Zn—O—C₁₋₂₀ alkyl, C₁₋₂₀ alkyl-O—Zn—O-heteroaryl, heteroaryl-O—Zn—O-heteroaryl, C₁₋₂₀ alkyl-O—Zn—O-heterocyclyl, heterocyclyl—O—Zn—O-heterocyclyl, and heterocyclyl—O—Zn—O-heteroaryl, wherein C₁₋₂₀ alkyl, heteroaryl, and heterocyclyl can be optionally substituted 1 to 3 times with R⁹;
R⁶ can be absent and, if present, is H or C₁₋₆ alkyl;
R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹¹, P(O)(OR¹⁰)(R¹¹)₂ P(O)(R¹¹)₂, BH₂, 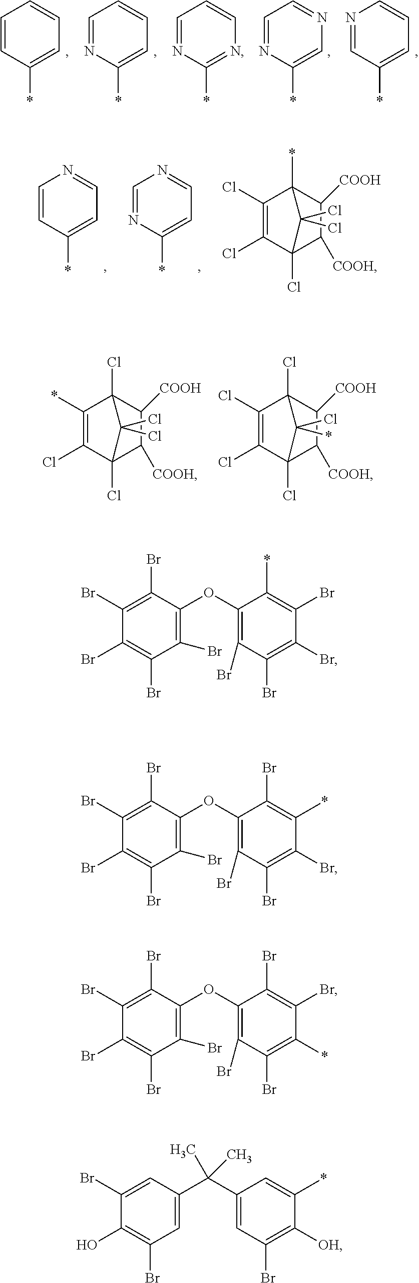 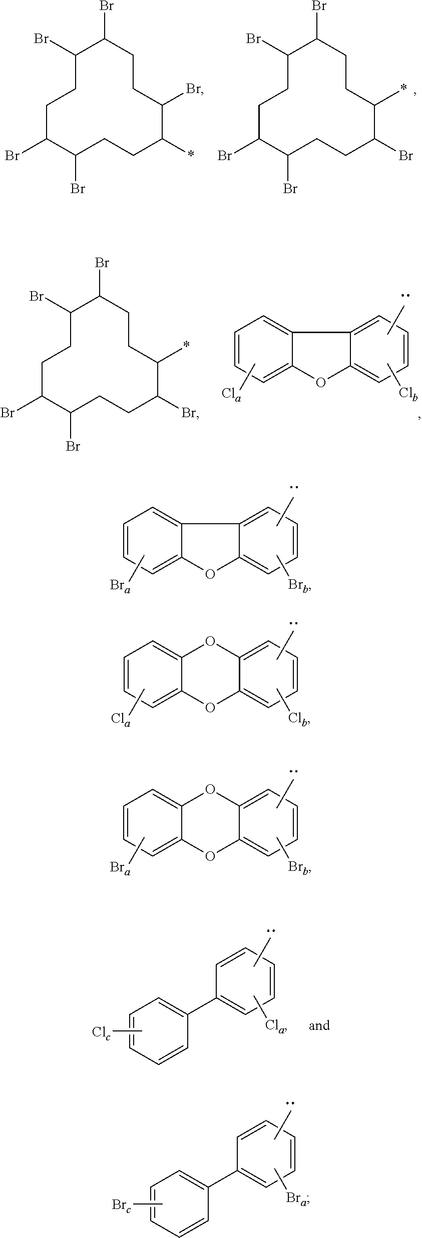
a is 1, 2, 3, or 4;
b is 1, 2, or 3;
c is 1, 2, 3, 4, or 5;
each R¹⁰ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl, [symbol] [symbol]
is the point of attachment of R⁹ to R⁵;
i is 1 to 1,000,000;
j is 1 to 1,000,000;
mis 0 to 32;
s is 0 to 32; and [symbol]
is a terminal group of the polymer, said
process comprises:
providing the compound of Formula (IVb) of claim 40;
providing a compound having the structure of Formula (XI): 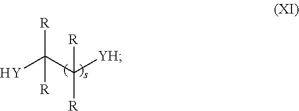
providing a compound having the structure of Formula (XII): 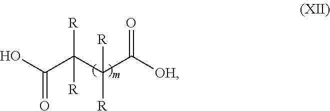
reacting the compound of Formula (IVb), the compound of Formula (XI), and the compound of Formula (XII) under conditions effective to produce the polymer of Formula (XVIII).

56. A process of making a polymer of Formula (XIII): 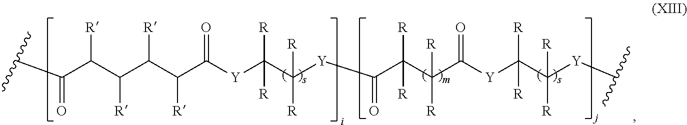
wherein
Y is NH or O;
R is independently selected from the group consisting of H and C₁₋₂₀ alkyl;
each R′ is independently H or 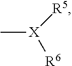
and only one of R′ is 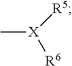
X is CH, O, S, N, P, or C═O;
R⁵ is selected from the group consisting of H, C₁₋₂₀ alkyl, heteroaryl, heterocyclyl, carborane, C₁₋₂₀ alkyl-O—Zn—O—C₁₋₂₀ alkyl, C₁₋₂₀ alkyl-O—Zn—O-heteroaryl, heteroaryl-O—Zn—O-heteroaryl, C₁₋₂₀ alkyl-O—Zn—O-heterocyclyl, heterocyclyl—O—Zn—O-heterocyclyl, and heterocyclyl—O—Zn—O-heteroaryl, wherein C₁₋₂₀ alkyl, heteroaryl, and heterocyclyl can be optionally substituted 1 to 3 times with R⁹;
R⁶ can be absent and, if present, is H or C₁₋₆ alkyl;
R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹¹, P(O)(OR¹⁰)(R¹¹)₂ P(O)(R¹¹)₂ BH₂, 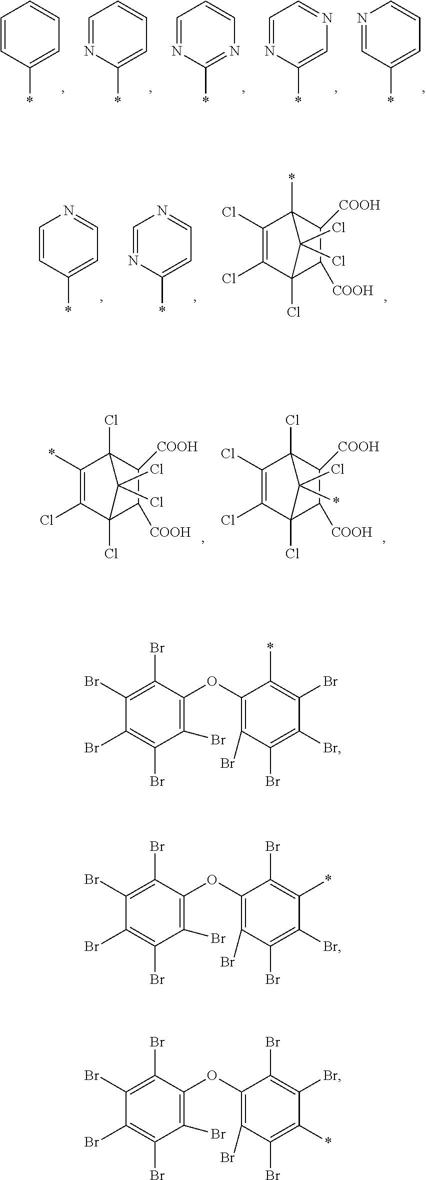 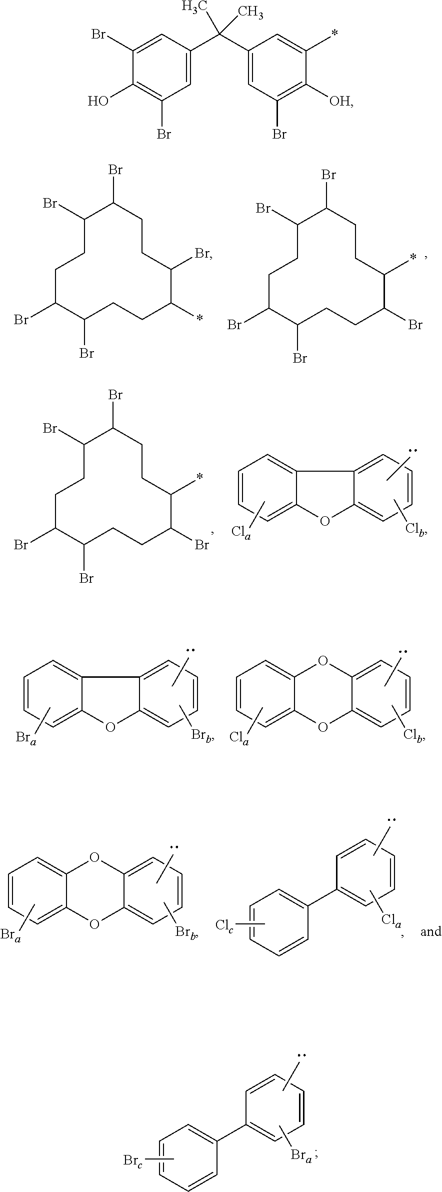
a is 1, 2, 3, or 4;
b is 1, 2, or 3;
c is 1, 2, 3, 4, or 5;
each R¹⁰ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl, [symbol]
is the point of attachment of R⁹ to R⁵;
i is 1 to 1,000,000;
j is 1 to 1,000,000;
m is 0 to 32;
s is 0 to 32; and [symbol]
is a terminal group of the polymer; [symbol]
is a single bond attached to one of the positions shown, said process comprises:
preparing the compound of Formula (IV) by the process of claim 13;
providing a compound having the structure of Formula (XI): 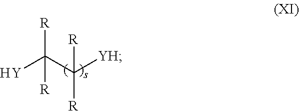
providing a compound having the structure of Formula (XII): 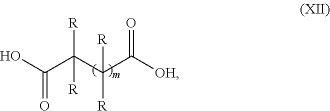
and
reacting the compound of Formula (IV), the compound of Formula (XI), and the compound of Formula (XII) under conditions effective to produce the polymer of Formula (XIII).

57-64. (canceled)

65. A polymer of Formula (XIII): 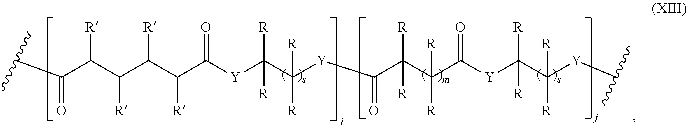
wherein
Y is NH or O;
R is independently selected from the group consisting of H and C₁₋₂₀ alkyl;
each R′ is independently H or 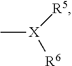
and only one of R′ is 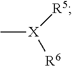
X is CH, O, S, N, P, or C═O;
R⁵ is selected from the group consisting of H, C₁₋₂₀ alkyl, heteroaryl, heterocyclyl, carborane, C₁₋₂₀ alkyl-O—Zn—O—C₁₋₂₀ alkyl, C₁₋₂₀ alkyl-O—Zn—O-heteroaryl, heteroaryl-O—Zn—O-heteroaryl, C₁₋₂₀ alkyl-O—Zn—O-heterocyclyl, heterocyclyl—O—Zn—O-heterocyclyl, and heterocyclyl—O—Zn—O-heteroaryl, wherein C₁₋₂₀ alkyl, heteroaryl, and heterocyclyl can be optionally substituted 1 to 3 times with R⁹;R⁶ can be absent and, if present, is H or C₁₋₆ alkyl;
R⁹ is independently selected from the group consisting of H, C₁₋₆ alkyl, SH, NH₂, PH₂, P(O)(OR¹⁰)₂, P(O)(OR¹⁰)₃, P(O)(OR¹⁰)₂R¹¹, P(O)(OR¹⁰)(R¹¹)₂ P(O)(R¹¹)₂ BH₂, 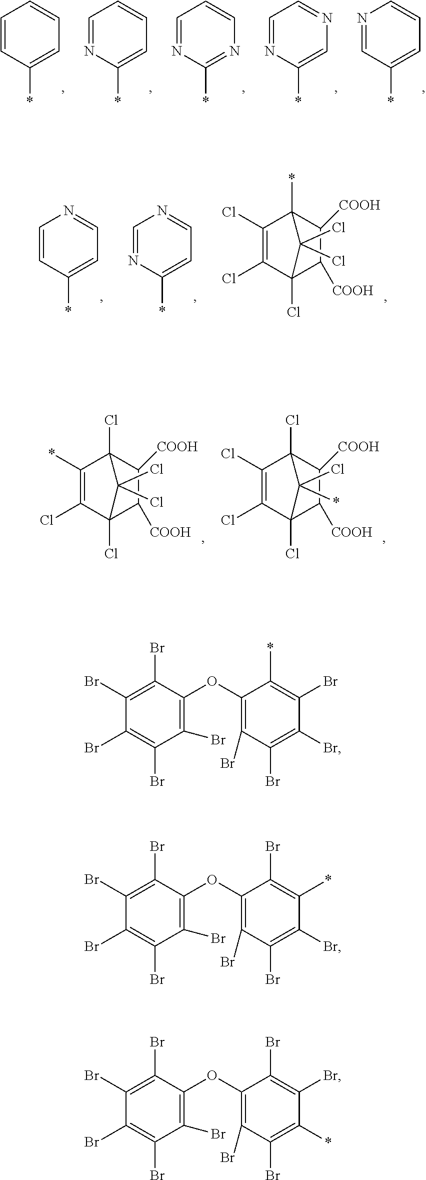 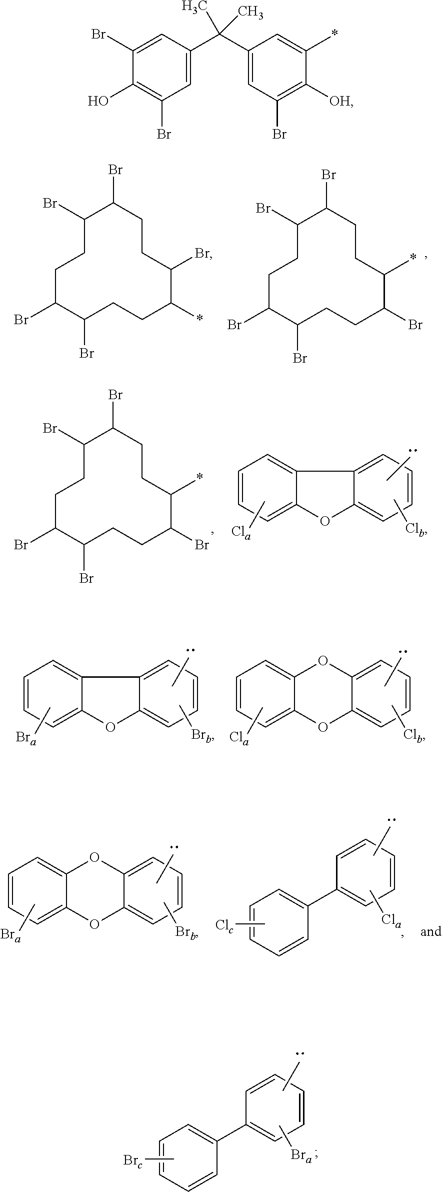
a is 1, 2, 3, or 4;
b is 1, 2, or 3;
c is 1, 2, 3, 4, or 5;
each R¹⁰ is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R¹¹ is independently selected from the group consisting of H, C₁₋₆ alkyl, C₁₋₆ alkenyl, and aryl, wherein aryl can be optionally substituted 1 to 3 times with halogen, or C₁₋₆ alkyl, [symbol]
is the point of attachment of R⁹ to R⁵;
i is 1 to 1,000,000;
j is 1 to 1,000,000;
m is 0 to 32;
s is 0 to 32; and [symbol]
is a terminal group of the polymer; [symbol]
is a single bond attached to one of the positions shown.

66. The polymer of claim 65, wherein the polymer has the structure of Formula (XIIIa), Formula (XIIIb), Formula (XIIIc), Formula (XIIId), Formula (XIIIe), Formula (XIIIf), Formula (XIIIg), or Formula (XIIIh):

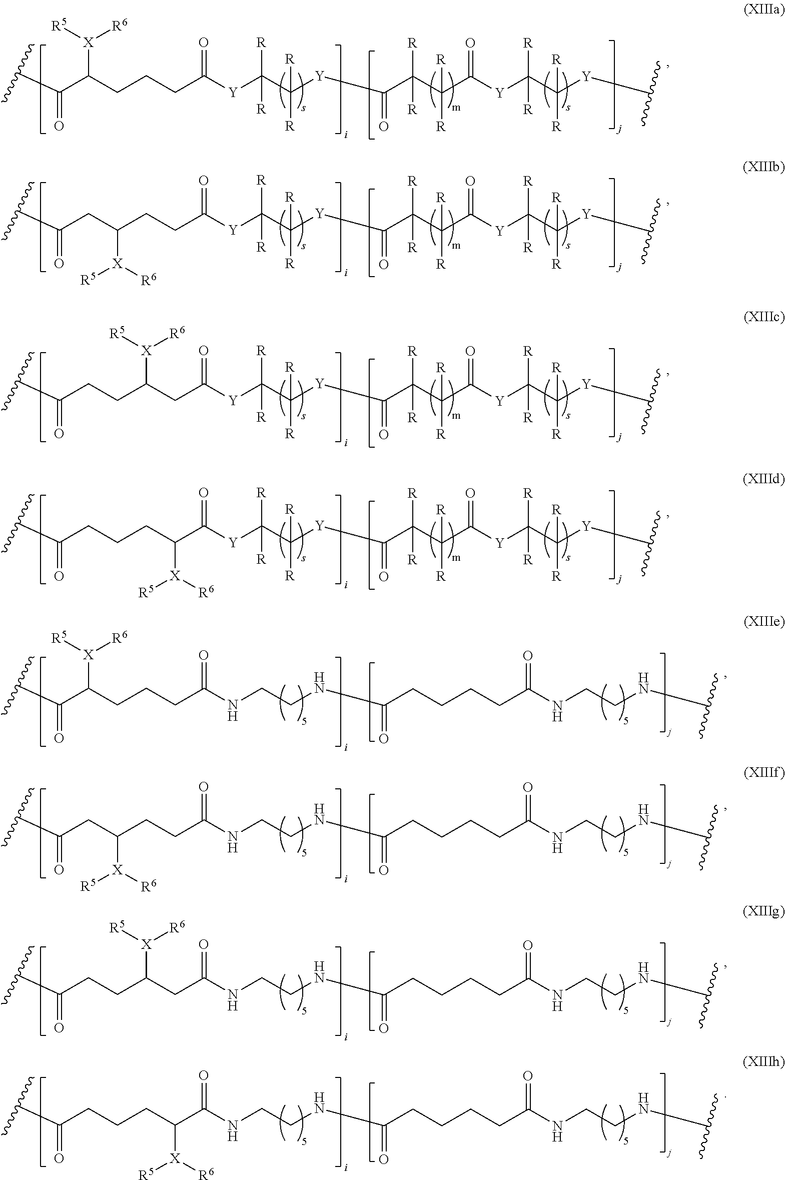

67-73. (canceled)

74. The polymer of claim 65, wherein the polymer is a statistical polymer.

75. The polymer of claim 65, wherein the polymer is a random polymer.

76. The polymer of claim 65, wherein the polymer is an alternating polymer.

77. The polymer of claim 65, wherein the polymer is a block polymer.

78. A process for preparation of a compound of Formula (XIV): 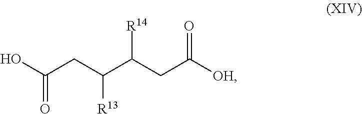
wherein
R¹³ is YR¹⁷R¹⁸;
R¹⁴ is ZR¹⁹R²⁰;
Y and Z are independently selected from CH, O, S, N, P, or C═O;
R¹⁷ and R¹⁹ are independently selected from the group consisting of H, C₁₋₂₀ alkyl, heteroaryl, heterocyclyl, carborane, C₁₋₂₀ alkyl-O—Zn—O C₁₋₂₀ alkyl, C₁₋₂₀ alkyl-O—Zn—O heteroaryl, heteroaryl-O—Zn—O-heteroaryl, C₁₋₂₀ alkyl-O—Zn—O-heterocyclyl, heterocyclyl O—Zn—O heterocyclyl, and heterocyclyl O—Zn—O heteroaryl, wherein C₁₋₂₀ alkyl, heteroaryl, and heterocyclyl can be independently optionally substituted 1 to 3 times with R²¹,
R¹⁸ and R²⁰ independently can be absent and, if present, are independently H or C₁₋₆ alkyl;
R²¹ is independently selected from the group consisting of H, C₁₋₆ alkyl, SH, NH2, PH₂, P(o)(OR²²)₂, P(O)(OR²²)₃, P(O)(OR²²)₂R²³, P(O)(OR²²)(R²³)₂, P(O)(R²³)₂, BH₂, 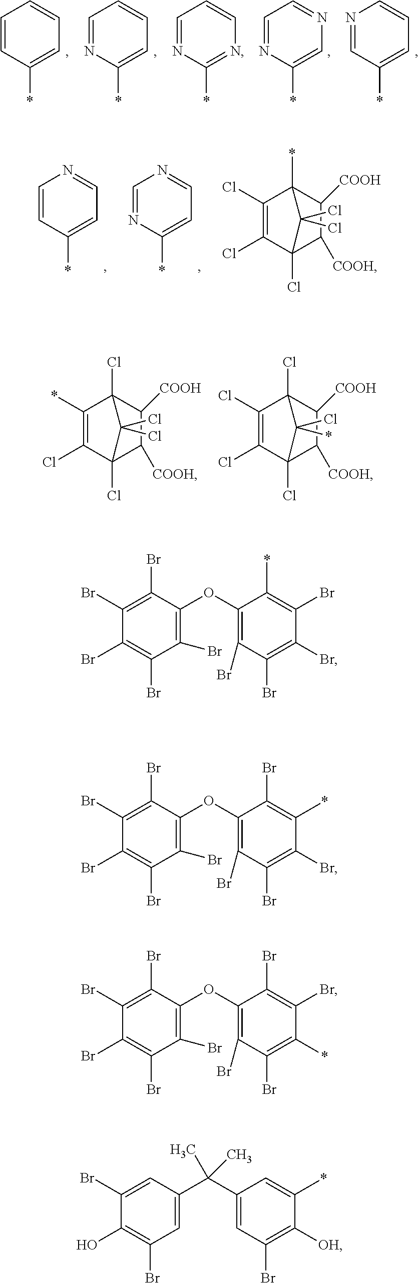 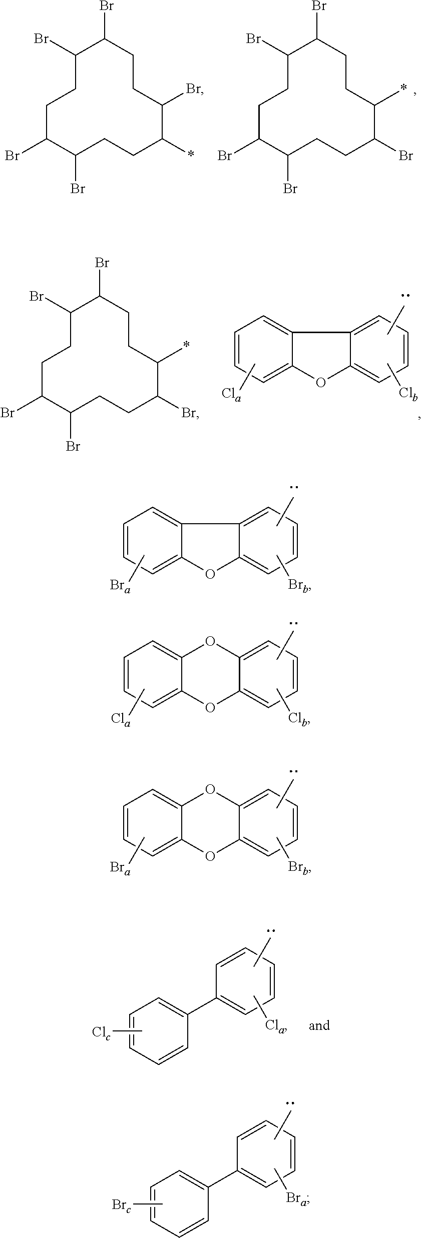
a is 1, 2, 3, or 4;
b is 1, 2, or 3;
c is 1, 2, 3, 4, or 5;
each R²² is independently selected from the group consisting of H, C₁₋₆ alkyl, C₁₋₆ alkenyl, and aryl, wherein aryl can be optionally substituted 1 to 3 times with halogen, or C₁₋₆ alkyl;
each R²³ is independently selected from the group consisting of H, C₁₋₆ alkyl, C₁₋₆ alkenyl, and aryl, wherein aryl can be optionally substituted 1 to 3 times with halogen, or C₁₋₆ alkyl, [symbol]
is the point of attachment of R²¹ to R¹⁷ or R¹⁹
or a salt thereof,
this process comprises:
providing a compound of Formula (XV) having the structure: 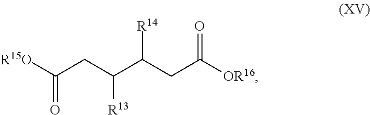
wherein
R¹⁵ is H or C₁₋₆ alkyl;
R¹⁶ is H or C₁₋₆ alkyl; and
forming the compound of Formula (I) from the compound of Formula (II).

79-91. (canceled)
